SEQUENCE LISTING (2 sequences)

SEQ ID NO: 1 - protein, 20 aa, synthetic construct
GGDYKDDDDKGGHHHHHHHH

SEQ ID NO: 2 - protein, 29 aa, synthetic construct
DYKDDDDKHHHHHHGLNDIFEAQKIEWHE

(19) United States
(12) Patent Application Publication
(10) Pub. No.: US 2025/0177394 A1
(43) Pub. Date: Jun. 5, 2025

(54) COMBINATIONS OF CTPS1 AND BCL2 INHIBITORS FOR CANCER

(71) Applicant: Step Pharma S.A.S., Paris Cedex 06 (FR)

(72) Inventors: Philip BEER, Paris Cedex 06 (FR); David CHIRON, Paris Cedex 06 (FR); Andrew PARKER, Paris Cedex 06 (FR); Catherine PELLAT-DECEUNYNCK, Paris Cedex 06 (FR)

(21) Appl. No.: 18/842,972

(22) PCT Filed: Mar. 1, 2023
(86) PCT No.: PCT/EP2023/055217

§ 371 Date: Aug. 30, 2024

(30) Foreign Application Priority Data
Mar. 1, 2022 (EP) 22305225.9
Sep. 27, 2022 (EP) 22306429.6

(51) Int. Cl. A61K 31/506; A61K 31/5377; A61K 31/635; A61P 35/00

(52) U.S. Cl. CPC A61K 31/506; A61K 31/5377; A61K 31/635; A61P 35/00

(57) ABSTRACT

The invention provides inter alia methods of treating cancer comprising administering to a subject a cytidine triphosphate synthase 1 (CTPS1) inhibitor and a B-cell lymphoma 2 (BCL2) inhibitor.

### FIELD OF THE INVENTION

The invention relates to combinations, in particular the combination of a CTPS1 inhibitor and a BCL2 inhibitor, pharmaceutical compositions and kits comprising such combinations which may be of use in the treatment of cancer and to related aspects.

### BACKGROUND OF THE INVENTION

Cancer can affect multiple cell types and tissues but the underlying cause is a breakdown in the control of cell division. This process is highly complex, requiring careful coordination of multiple pathways, many of which remain to be fully characterised. Cell division requires the effective replication of the cell's DNA and other constituents. Interfering with a cell's ability to replicate by targeting nucleic acid synthesis has been a core approach in cancer therapy for many years. Examples of therapies acting in this way are 6-thioguanine, 6-mecaptopurine, 5-fluorouracil, cytarabine, gemcitabine and pemetrexed.

Cancer therapeutics against a wide array of specific targets are available. Small molecule targeted therapy drugs are generally inhibitors of enzymatic domains on mutated, overexpressed, or otherwise critical proteins within the cancer cell. Monoclonal antibody therapy is another strategy in which the therapeutic agent is an antibody which specifically binds to a protein on the surface of the cancer cells.

All proliferating cells, including neoplastic cells, are reliant on a ready source of purine and pyrimidine nucleotides for DNA and RNA synthesis. Whilst salvage pathways may be sufficient for steady state metabolism, DNA replication to enable cell division is dependent on synthesis of nucleotides via the de novo pathway. A key bottleneck in the de novo pyrimidine synthesis pathway is the enzyme cytidine triphosphate synthase (CTPS) which catalyses the conversion of UTP to CTP (van Kuilenburg 2000). CTPS also has two isoforms in humans (CTPS1 and CTPS2; see FIG. 1). Both isoforms are ubiquitously expressed in normal and malignant human cells (BioGPS and EMBL-EBI Expression Atlas). Human genetic studies have identified an essential and non-redundant role for CTPS1 in the proliferation of normal immune (B and T) cells (Martin 2014; Martin 2020).

Whilst cancer cells are dependent on CTPS activity in order to proliferate, the precise role that CTPS1 and CTPS2 play in cancer is currently not completely clear. Several CTPS inhibitors that inhibit both CTPS1 and CTPS2 have been developed for oncology indications up to phase I/II clinical trials, but were stopped due to toxicity and efficacy issues. Most of the developed inhibitors are nucleoside-analogue prodrugs (3-deazauridine (DAU), CPEC, carbodine, gemcitabine), which are converted to the active triphosphorylated metabolite by the kinases involved in pyrimidine biosynthesis: uridine/cytidine kinase, nucleoside monophosphate-kinase (NMP-kinase) and nucleoside diphosphatekinase (NDP-kinase). The remaining inhibitors (acivicin, DON) are reactive analogues of glutamine, which irreversibly inhibit the glutaminase domain of CTPS. Importantly, none of the inhibitors of CTPS developed to date are selective for one isoform of CTPS over the other. As such, available CTPS inhibitors block all CTPS activity and, therefore, block the ability of all cells in the body to undergo cell division.

BCL2 is a member of an important family of pro-survival, antiapoptotic proteins that are commonly expressed in cancer cells. Inhibition of BCL2 activity can result in the triggering of apoptosis, resulting in cell death. This is mediated through the mitochondrion, whereby pro-apoptotic proteins form complexes which make pores in the mitochondrial membrane resulting in mitochondrial depolarisation which triggers apoptosis. Therapeutic inhibition of BCL2 can be achieved by drugs designed to mimic the activity of the BH3 domain of pro-apoptotic proteins. Therapeutic agents using this approach are referred to as BH3-mimetics, which induce apoptosis by triggering mitochondrial membrane depolarisation (Warren 2019; Diepstraten 2022).

BCL2 inhibition has shown utility in haematological cancers, most notably chronic lymphocytic leukaemia and acute myeloid leukaemia (Roberts 2021). Clinical trials of BCL2 inhibitors are underway in solid tumour indications.

There remains a need for new approaches to cancer therapies, such approaches may demonstrate high in vivo efficacy, reduction in the dose required for effect in vivo, an improved safety profile/reduced side effects, or the like.

### SUMMARY OF THE INVENTION

The invention provides a CTPS1 inhibitor for use in the treatment of cancer with a BCL2 inhibitor.

In a further aspect the invention provides a BCL2 inhibitor for use in the treatment of cancer with a CTPS1 inhibitor.

In a further aspect the invention provides a CTPS1 inhibitor and a BCL2 inhibitor for use in the treatment of cancer.

In a further aspect the invention provides the use of a CTPS1 inhibitor in the manufacture of a medicament for the treatment of cancer with a BCL2 inhibitor.

In a further aspect the invention provides the use of a BCL2 inhibitor in the manufacture of a medicament for the treatment of cancer with a CTPS1 inhibitor.

In a further aspect the invention provides the use of a CTPS1 inhibitor and a BCL2 inhibitor in the manufacture of a medicament for the treatment of cancer.

In a further aspect the invention provides a method of treating cancer in a subject which method comprises administering to the subject a CTPS1 inhibitor and a BCL2 inhibitor.

In a further aspect the invention provides a pharmaceutical composition comprising a CTPS1 inhibitor and a BCL2 inhibitor.

In a further aspect the invention provides a kit of parts comprising:
a) a first container comprising a CTPS1 inhibitor; and
b) a second container comprising a BCL2 inhibitor.

### Summary of the Sequences

SEQ ID NO: 1 FLAG-His₈-tag

SEQ ID NO: 2 FLAG-His-Avi tag

### SUMMARY OF THE FIGURES

FIG. 1 De novo CTP production pathway

FIG. 2 Impact of deletion of different genes in the pyrimidine synthesis pathway

FIG. 3 Bliss scores for the interaction between CTPS1-IA and venetoclax for a range of cell lines derived from B cell malignancies

FIG. 4 Bliss scores for the interaction between CTPS1-IA and venetoclax for a range of cell lines derived from solid tumours

FIG. 5 Bliss scores for the interaction between CTPS1-IA and BCL201 for a range of cell lines derived from solid tumours

FIG. 6 Bliss scores for the interaction between CTPS1-IA and venetoclax for a range of cell lines derived from human T cell lymphomas

FIG. 7 In vivo tumour growth studies performed investigating the effects of CTPS1-IA and venetoclax using a human cancer cell line

FIG. 8 Levels of RNA transcripts measured in human mantle cell lymphoma cell lines following a 24 hour exposure to either CTPS1-IA or control, showing an increase in BCL2 transcripts and a decrease in MCL1 transcripts following exposure to CTPS1-IA. * P<0.05, paired t-test

FIG. 9 Protein levels assessed by western blot analysis from a human mantle cell lymphoma cell line following a 24 hour exposure to either CTPS1-IA or control, showing an increase in BCL2 protein and a decrease in MCL1 protein following CTPS1-IA exposure; GAPDH is shown as a protein loading control

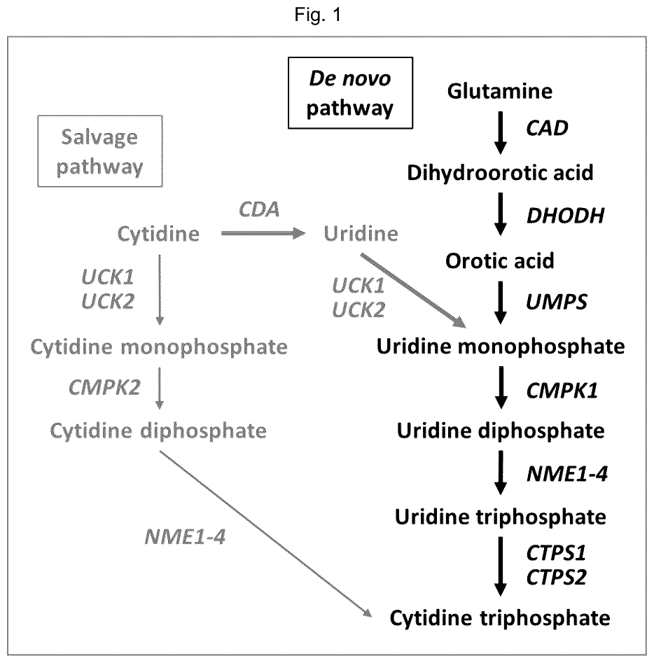

### DETAILED DESCRIPTION OF THE INVENTION

### CTPS1 Inhibitors

In one aspect of the invention there is provided a CTPS1 inhibitor for use in the treatment of cancer with a BCL2 inhibitor.

A CTPS1 inhibitor, as used herein, is an agent which directly inhibits the enzymatic activity of the CTPS1 enzyme through interaction with the enzyme. Direct inhibition of the CTPS1 enzyme may be quantified using any suitable assay procedure, though is suitably performed using the procedure set out in Example 1.

CTPS1 inhibitors may demonstrate an IC₅₀ of 10 uM or lower, such as 1 uM or lower, especially 100 nM or lower, in respect of CTPS1 enzyme. CTPS1 inhibitors of particular interest are those demonstrating an IC₅₀ of 10 uM or lower, such as 1 uM or lower, especially 100 nM or lower, in respect of CTPS1 enzyme using the assay procedure set out in Example 1.

CTPS1 inhibitors may demonstrate a selectivity for CTPS1 over CTPS2. Suitably the inhibitors demonstrate a selectivity of at least 2-fold, such as at least 30-fold, especially at least 60-fold and in particular at least 1000-fold. CTPS1 inhibitors of particular interest are those demonstrating a selectivity for CTPS1 over CTPS2, suitably of at least 2-fold, such as at least 30-fold, especially at least 60-fold and in particular at least 1000-fold using the assay procedure set out in Example 2. Desirably the selectivity is for human CTPS1 over human CTPS2.

In the case of medicaments intended for human use, CTPS1 inhibition and CTPS1 vs CTPS2 selectivity should be based on human forms of the enzymes.

Suitably the CTPS1 inhibitor may be selected from the following compounds:

A compound of formula (I):

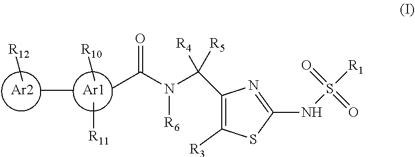

wherein
R₁ is C₁₋₅alkyl, C₀₋₂alkyleneC₃₋₅cycloalkyl which cycloalkyl is optionally substituted by
CH₃, C₁₋₃alkyleneOC₁₋₂alkyl, or CF₃;
R₃ is H, CH₃, halo, OC₁₋₂alkyl or CF₃;
R₄ and R₅ are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl, or R₄ and R₅ together with the carbon atom to which they are attached form a C₃₋₆cycloalkyl or C₃₋₆heterocycloalkyl ring;
R₆ is H or C₁₋₃alkyl;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, C, CH₃, ethyl, OCH₃, CF₃, OCF₃ or CN;
R₁₂ is attached to Ar2 in the meta or ortho position relative to Ar1 and R₁₂ is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR₂₃R₂₄, SO₂CH₃, C(O)N(CH₃)₂, NHC(O)C₁₋₃alkyl, or a C₃₋₆heterocycloalkyl comprising one nitrogen located at the point of attachment to
Ar2, or R₁₂ together with a nitrogen atom to which it is attached forms an N-oxide (N⁺—O⁻);
R₂₃ is H or C₁₋₂alkyl;
R₂₄ is H or C₁₋₂alkyl;
or a pharmaceutically acceptable salt and/or pharmaceutically acceptable solvate thereof.

More suitably the CTPS1 inhibitor is selected from the following (‘List A’) compounds:
N-((2-(cyclopropanesulfonamido)thiazol-4-yl)methyl)-5-phenylpicolinamide;
N-((2-(cyclopropanesulfonamido)thiazol-4-yl)methyl)-4-(pyridin-3-yl)benzamide;
N-(1-(2-(cyclopropanesulfonamido)thiazol-4-yl)propyl)-4-(5-(trifluoromethyl)pyridin-3-yl)benzamide;
N-(1-(2-(cyclopropanesulfonamido)thiazol-4-yl)propyl)-4-(5-(trifluoromethyl)pyridin-3-yl)benzamide (R enantiomer);
N-(1-(2-(cyclopropanesulfonamido)thiazol-4-yl)propyl)-4-(5-(trifluoromethyl)pyridin-3-yl)benzamide (S enantiomer);
N-(1-(2-(cyclopropanesulfonamido)thiazol-4-yl)propyl)-4-(6-(trifluoromethyl)pyrazin-2-yl)benzamide;
N-(2-(2-(cyclopropanesulfonamido)thiazol-4-yl)propan-2-yl)-4-(6-ethoxypyrazin-2-yl)-2-fluorobenzamide;
N-(2-(2-(cyclopropanesulfonamido)thiazol-4-yl)propan-2-yl)-4-(6-ethoxypyrazin-2-yl)-2-methoxybenzamide;
N-((2-(cyclopropanesulfonamido)thiazol-4-yl)methyl)-[1,1′-biphenyl]-4-carboxamide;
N-((2-(cyclopropanesulfonamido)thiazol-4-yl)methyl)-2-fluoro-4-(6-(trifluoromethyl)pyrazin-2-yl)benzamide;
N-((2-(cyclopropanesulfonamido)thiazol-4-yl)methyl)-4-(6-ethoxypyrazin-2-yl)-2-fluorobenzamide;
N-((2-(cyclopropanesulfonamido)thiazol-4-yl)methyl)-4-(6-(trifluoromethyl)pyrazin-2-yl)benzamide;
N-((2-(cyclopropanesulfonamido)thiazol-4-yl)methyl)-4-(6-isopropoxypyrazin-2-yl)benzamide;
N-((2-(cyclopropanesulfonamido)thiazol-4-yl)methyl)-4-(6-ethoxypyrazin-2-yl)benzamide;
N-(3-(2-(cyclopropanesulfonamido)thiazol-4-yl)pentan-3-yl)-4-(5-(trifluoromethyl)pyridin-3-yl)benzamide;
N-(3-(2-(cyclopropanesulfonamido)thiazol-4-yl)pentan-3-yl)-4-(5-fluoropyridin-3-yl)benzamide;
N-(3-(2-(cyclopropanesulfonamido)thiazol-4-yl)pentan-3-yl)-4-(5-methylpyridin-3-yl)benzamide;
N-(3-(2-(cyclopropanesulfonamido)thiazol-4-yl)pentan-3-yl)-4-(pyridin-3-yl)benzamide;
N-(3-(2-(cyclopropanesulfonamido)thiazol-4-yl)pentan-3-yl)-4-(6-(trifluoromethyl)pyrazin-2-yl)benzamide;
4-(6-chloropyrazin-2-yl)-N-(3-(2-(cyclopropanesulfonamido)thiazol-4-yl)pentan-3-yl)benzamide;
N-(3-(2-(cyclopropanesulfonamido)thiazol-4-yl)pentan-3-yl)-4-(6-methylpyrazin-2-yl)benzamide;
N-(3-(2-(cyclopropanesulfonamido)thiazol-4-yl)pentan-3-yl)-4-(pyrazin-2-yl)benzamide;
N-(2-(2-(cyclopropanesulfonamido)thiazol-4-yl)propan-2-yl)-5-(6-ethoxypyrazin-2-yl)-3-fluoropicolinamide;
N-(2-(2-(cyclopropanesulfonamido)thiazol-4-yl)propan-2-yl)-5-(6-(trifluoromethyl)pyrazin-2-yl)picolinamide;
5-(6-chloropyrazin-2-yl)-N-(2-(2-(cyclopropanesulfonamido)thiazol-4-yl)propan-2-yl)picolinamide;
N-(2-(2-(cyclopropanesulfonamido)thiazol-4-yl)propan-2-yl)-5-(6-ethoxypyrazin-2-yl)picolinamide;
N-(2-(2-(cyclopropanesulfonamido)thiazol-4-yl)propan-2-yl)-[2,2′-bipyridine]-5-carboxamide;
4-(5-chloropyridin-3-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-2-fluoro-4-(5-(trifluoromethyl)pyridin-3-yl)benzamide;
4-(5-chloropyridin-3-yl)-N-(2-(2-(cyclopropanesulfonamido)thiazol-4-yl)propan-2-yl)-2-fluorobenzamide;
N-(2-(2-(cyclopropanesulfonamido)thiazol-4-yl)propan-2-yl)-2-fluoro-4-(5-fluoropyridin-3-yl)benzamide;
N-(2-(2-(cyclopropanesulfonamido)thiazol-4-yl)propan-2-yl)-2-methoxy-4-(5-(trifluoromethyl)pyridin-3-yl)benzamide;
4-(5-acetylpyridin-3-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-4-(5-(trifluoromethyl)pyridin-3-yl)benzamide;
N-(2-(2-(cyclopropanesulfonamido)thiazol-4-yl)propan-2-yl)-4-(5-fluoropyridin-3-yl)benzamide;
N-(2-(2-(cyclopropanesulfonamido)thiazol-4-yl)propan-2-yl)-4-(5-methylpyridin-3-yl)benzamide;
N-(2-(2-(cyclopropanesulfonamido)thiazol-4-yl)propan-2-yl)-4-(5-methoxypyridin-3-yl)benzamide;
N-(2-(2-(cyclopropanesulfonamido)thiazol-4-yl)propan-2-yl)-4-(pyridin-3-yl)benzamide;
N-(2-(2-(cyclopropanesulfonamido)thiazol-4-yl)propan-2-yl)-3′-(trifluoromethyl)-[1,1′-biphenyl]-4-carboxamide;
N-(2-(2-(cyclopropanesulfonamido)thiazol-4-yl)propan-2-yl)-4-(6-ethylpyrazin-2-yl)-2-fluorobenzamide;
N-(2-(2-(cyclopropanesulfonamido)thiazol-4-yl)propan-2-yl)-2-fluoro-4-(6-(trifluoromethyl)pyrazin-2-yl)benzamide;
N-(2-(2-(cyclopropanesulfonamido)thiazol-4-yl)propan-2-yl)-2-fluoro-4-(6-isopropoxypyrazin-2-yl)benzamide;
N-(2-(2-(cyclopropanesulfonamido)thiazol-4-yl)propan-2-yl)-2-fluoro-4-(6-(2,2,2-trifluoroethoxy)pyrazin-2-yl)benzamide;
N-(2-(2-(cyclopropanesulfonamido)thiazol-4-yl)propan-2-yl)-2-methyl-4-(6-(trifluoromethyl)pyrazin-2-yl)benzamide;
N-(2-(2-(cyclopropanesulfonamido)thiazol-4-yl)propan-2-yl)-4-(6-ethoxypyrazin-2-yl)-2-methylbenzamide;
N-(2-(2-(cyclopropanesulfonamido)thiazol-4-yl)propan-2-yl)-4-(6-ethoxypyrazin-2-yl)-2-(trifluoromethyl)benzamide;
N-(2-(2-(cyclopropanesulfonamido)thiazol-4-yl)propan-2-yl)-2-methoxy-4-(6-(trifluoromethyl)pyrazin-2-yl)benzamide;
4-(6-chloropyrazin-2-yl)-N-(2-(2-(cyclopropanesulfonamido)thiazol-4-yl)propan-2-yl)-2-methoxybenzamide;
4-(6-cyanopyrazin-2-yl)-N-(2-(2-(cyclopropanesulfonamido)thiazol-4-yl)propan-2-yl)-2-methoxybenzamide;
N-(2-(2-(cyclopropanesulfonamido)thiazol-4-yl)propan-2-yl)-4-(6-(trifluoromethyl)pyrazin-2-yl)benzamide;
4-(6-chloropyrazin-2-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-4-(6-methylpyrazin-2-yl)benzamide;
N-(2-(2-(cyclopropanesulfonamido)thiazol-4-yl)propan-2-yl)-4-(6-methoxypyrazin-2-yl)benzamide;
N-(2-(2-(cyclopropanesulfonamido)thiazol-4-yl)propan-2-yl)-4-(6-ethoxypyrazin-2-yl)benzamide;
N-(2-(2-(cyclopropanesulfonamido)thiazol-4-yl)propan-2-yl)-4-(6-isopropoxypyrazin-2-yl)benzamide;
N-(2-(2-(cyclopropanesulfonamido)thiazol-4-yl)propan-2-yl)-4-(6-(2,2,2-trifluoroethoxy)pyrazin-2-yl)benzamide;
N-(2-(2-(cyclopropanesulfonamido)thiazol-4-yl)propan-2-yl)-4-(pyrazin-2-yl)benzamide;
N-(1-(2-(cyclopropanesulfonamido)thiazol-4-yl)propyl)-4-(5-fluoropyridin-3-yl)benzamide;
N-(1-(2-(cyclopropanesulfonamido)thiazol-4-yl)propyl)-4-(5-methylpyridin-3-yl)benzamide;
N-(1-(2-(cyclopropanesulfonamido)thiazol-4-yl)propyl)-4-(pyridin-3-yl)benzamide;
N-(1-(2-(cyclopropanesulfonamido)thiazol-4-yl)propyl)-4-(6-ethoxypyrazin-2-yl)-2-fluorobenzamide;
N-(1-(2-(cyclopropanesulfonamido)thiazol-4-yl)propyl)-4-(6-ethoxypyrazin-2-yl)-2-fluoro-N-methylbenzamide;
N-(1-(2-(cyclopropanesulfonamido)thiazol-4-yl)propyl)-2-fluoro-4-(6-isopropoxypyrazin-2-yl)benzamide;
4-(6-chloropyrazin-2-yl)-N-(1-(2-(cyclopropanesulfonamido)thiazol-4-yl)propyl)benzamide;
N-(1-(2-(cyclopropanesulfonamido)thiazol-4-yl)propyl)-4-(6-methylpyrazin-2-yl)benzamide;
N-(1-(2-(cyclopropanesulfonamido)thiazol-4-yl)propyl)-4-(pyrazin-2-yl)benzamide;
N-(1-(2-(cyclopropanesulfonamido)thiazol-4-yl)propyl)-4-(5-fluoropyridin-3-yl)benzamide (R enantiomer);
N-(1-(2-(cyclopropanesulfonamido)thiazol-4-yl)propyl)-4-(5-fluoropyridin-3-yl)benzamide (S enantiomer);
N-(1-(2-(cyclopropanesulfonamido)thiazol-4-yl)propyl)-4-(6-ethoxypyrazin-2-yl)-2-fluorobenzamide (R enantiomer);
N-(1-(2-(cyclopropanesulfonamido)thiazol-4-yl)propyl)-4-(6-ethoxypyrazin-2-yl)-2-fluorobenzamide (S enantiomer);
N-(2-(2-(cyclopropanesulfonamido)-5-methylthiazol-4-yl)propan-2-yl)-5-(6-ethoxypyrazin-2-yl)picolinamide;
N-(2-(5-chloro-2-(cyclopropanesulfonamido)thiazol-4-yl)propan-2-yl)-5-(6-ethoxypyrazin-2-yl)picolinamide;
N-(2-(2-(cyclopropanesulfonamido)-5-methylthiazol-4-yl)propan-2-yl)-4-(6-ethoxypyrazin-2-yl)-2-fluorobenzamide;
N-(2-(5-chloro-2-(cyclopropanesulfonamido)thiazol-4-yl)propan-2-yl)-4-(6-ethoxypyrazin-2-yl)-2-fluorobenzamide;
N-(2-(2-(cyclopropanesulfonamido)-5-methylthiazol-4-yl)propan-2-yl)-2-methyl-4-(6-(trifluoromethyl)pyrazin-2-yl)benzamide;
N-(2-(5-chloro-2-(cyclopropanesulfonamido)thiazol-4-yl)propan-2-yl)-2-methyl-4-(6-(trifluoromethyl)pyrazin-2-yl)benzamide;
N-(2-(2-(cyclopropanesulfonamido)-5-methylthiazol-4-yl)propan-2-yl)-4-(6-(trifluoromethyl)pyrazin-2-yl)benzamide;
N-(2-(5-chloro-2-(cyclopropanesulfonamido)thiazol-4-yl)propan-2-yl)-4-(6-(trifluoromethyl)pyrazin-2-yl)benzamide;
N-(1-(2-(cyclopropanesulfonamido)thiazol-4-yl)cyclopropyl)-5-(6-ethoxypyrazin-2-yl)picolinamide;
N-(1-(2-(cyclopropanesulfonamido)thiazol-4-yl)cyclopropyl)-4-(pyridin-3-yl)benzamide;
N-(1-(2-(cyclopropanesulfonamido)thiazol-4-yl)cyclopropyl)-4-(6-ethoxypyrazin-2-yl)-2-fluorobenzamide;
N-(1-(2-(cyclopropanesulfonamido)thiazol-4-yl)cyclopropyl)-2-methyl-4-(6-(trifluoromethyl)pyrazin-2-yl)benzamide;
N-(1-(2-(cyclopropanesulfonamido)thiazol-4-yl)cyclopropyl)-4-(6-(trifluoromethyl)pyrazin-2-yl)benzamide;
N-(1-(2-(cyclopropanesulfonamido)thiazol-4-yl)-3-methoxypropyl)-4-(5-fluoropyridin-3-yl)benzamide;
N-(1-(2-(cyclopropanesulfonamido)thiazol-4-yl)-3-methoxypropyl)-4-(6-ethylpyrazin-2-yl)-2-fluorobenzamide;
N-(1-(2-(cyclopropanesulfonamido)thiazol-4-yl)-3-methoxypropyl)-2-fluoro-4-(6-(trifluoromethyl)pyrazin-2-yl)benzamide;
N-(1-(2-(cyclopropanesulfonamido)thiazol-4-yl)-3-methoxypropyl)-4-(6-ethoxypyrazin-2-yl)-2-fluorobenzamide;
N-(1-(2-(cyclopropanesulfonamido)thiazol-4-yl)-3-methoxypropyl)-2-fluoro-4-(6-isopropoxypyrazin-2-yl)benzamide;
N-(1-(2-(cyclopropanesulfonamido)thiazol-4-yl)-3-methoxypropyl)-4-(6-ethoxypyrazin-2-yl)benzamide;
N-(1-(2-(cyclopropanesulfonamido)thiazol-4-yl)ethyl)-4-(6-ethoxypyrazin-2-yl)-2-fluorobenzamide;
N-(1-(2-(cyclopropanesulfonamido)thiazol-4-yl)-3-methoxypropyl)-4-(6-ethoxypyrazin-2-yl)-2-fluorobenzamide (R enantiomer); and
N-(1-(2-(cyclopropanesulfonamido)thiazol-4-yl)-3-methoxypropyl)-4-(6-ethoxypyrazin-2-yl)-2-fluorobenzamide (S enantiomer);
   or a pharmaceutically acceptable salt and/or pharmaceutically acceptable solvate thereof.

Such CTPS1 inhibitors are disclosed in PCT publication number WO2019106146 which is incorporated by reference in its entirety for the purpose of the CTPS1 inhibitors disclosed therein. In particular a CTPS1 inhibitor may be a compound described in any one of clauses 1 to 110 of WO2019106146 or a pharmaceutically acceptable salt and/or pharmaceutically acceptable solvate thereof, in particular a compound R1 to R93 or a pharmaceutically acceptable salt and/or pharmaceutically acceptable solvate thereof.

Alternatively, the CTPS1 inhibitor is compound of formula (II):

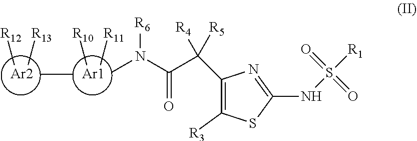

wherein
R₁ is C₁₋₅alkyl, C₀₋₂alkyleneC₃₋₅cycloalkyl which cycloalkyl is optionally substituted by
CH₃, C₁₋₃alkyleneOC₁₋₂alkyl, or CF₃;
R₃ is H, halo, CH₃, OC₁₋₂alkyl or CF₃;
   or R₃ together with R₅ forms a 5- or 6-membered cycloalkyl or 5 or 6 membered oxygen-containing heterocycloalkyl;
R₄ and R₅ are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR₂₁R₂₂,
   or R₄ is H and R₅ together with R₃ form a 5- or 6-membered cycloalkyl or 5 or 6 membered oxygen-containing heterocycloalkyl,
   or R₄ and R₅ together with the carbon atom to which they are attached form a C₃₋₆cycloalkyl or C₃₋₆heterocycloalkyl,
   or R₄ is H and R₅ and R₆ are a C₂₋₃alkylene chain forming a 5- or 6-membered ring;
   or R₄ is 0 and R₅ is absent;
R₆ is H or C₁₋₃alkyl,
   or R₆ together with R₁₁ when in the ortho-position to the amide are a C₂alkylene chain forming a 5-membered ring,
   or R₅ and R₆ are a C₂₋₃alkylene chain forming a 5- or 6-membered ring and R₄ is H;
Ar1 is 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, Cl, CH₃, ethyl, OCH₃, CF₃, OCF₃ or CN,
   or R₁₁, when in the ortho-position to the amide, together with R₆ are a C₂alkylene chain forming a 5-membered ring;
R₁₂ is attached to Ar2 in the ortho or meta position relative to Ar1 and R₁₂ is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR₂₃R₂₄, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H, halo, CH₃ or OCH₃;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
   or a pharmaceutically acceptable salt and/or pharmaceutically acceptable solvate thereof.

More suitably the CTPS1 inhibitor is selected from the following (‘List B’) compounds:
N-([1,1′-biphenyl]-4-yl)-2-(2-(methylsulfonamido)thiazol-4-yl)acetamide;
N-([1,1′-biphenyl]-4-yl)-2-(2-(cyclopropanesulfonamido)thiazol-4-yl)acetamide;
2-(2-(cyclopropanesulfonamido)thiazol-4-yl)-2-ethyl-N-(5-(pyrazin-2-yl)pyridin-2-yl)butanamide;
2-(2-(cyclopropanesulfonamido)thiazol-4-yl)-2-methyl-N-(4-(pyrimidin-2-yl)phenyl)propanamide;
2-(2-(cyclopropanesulfonamido)thiazol-4-yl)-N-(4-(pyridin-3-yl)phenyl)butanamide (racemic);
(R)-2-(2-(cyclopropanesulfonamido)thiazol-4-yl)-N-(4-(pyridin-3-yl)phenyl)butanamide;
(S)-2-(2-(cyclopropanesulfonamido)thiazol-4-yl)-N-(4-(pyridin-3-yl)phenyl)butanamide;
2-(2-(cyclopropanesulfonamido)thiazol-4-yl)-N-(4-(5-fluoropyridin-3-yl)phenyl)butanamide (racemic);
(R)-2-(2-(cyclopropanesulfonamido)thiazol-4-yl)-N-(4-(5-fluoropyridin-3-yl)phenyl)butanamide;
(S)-2-(2-(cyclopropanesulfonamido)thiazol-4-yl)-N-(4-(5-fluoropyridin-3-yl)phenyl)butanamide;
2-(2-(cyclopropanesulfonamido)thiazol-4-yl)-3-methyl-N-(4-(pyrimidin-5-yl)phenyl)butanamide;
2-(2-(cyclopropanesulfonamido)thiazol-4-yl)-3-methyl-N-(4-(pyridin-3-yl)phenyl)butanamide;
2-(2-(cyclopropanesulfonamido)thiazol-4-yl)-N-(4-(5-methoxypyridin-3-yl)phenyl)-2-methylpropanamide;
N-(2-chloro-4-(pyridin-3-yl)phenyl)-2-(2-(cyclopropanesulfonamido)thiazol-4-yl)acetamide;
2-(2-(cyclopropanesulfonamido)-5-methylthiazol-4-yl)-2-methyl-N-(4-(6-(trifluoromethyl)pyrazin-2-yl)phenyl)propanamide;
2-(2-(cyclopropanesulfonamido)thiazol-4-yl)-2-methyl-N-(4-(pyrimidin-5-yl)phenyl)propanamide;
6-(4-(2-(2-(cyclopropanesulfonamido)thiazol-4-yl)-2-methylpropanamido)phenyl)-N,N-dimethylpyrazine-2-carboxamide;
N-(5-(5-cyanopyridin-3-yl)pyrimidin-2-yl)-2-(2-(cyclopropanesulfonamido)thiazol-4-yl)-2-methylpropanamide;
N-([1,1′-biphenyl]-4-yl)-2-(5-chloro-2-(cyclopropanesulfonamido)thiazol-4-yl)acetamide;
2-(2-(cyclopropanesulfonamido)thiazol-4-yl)-N-(4-(6-ethynylpyrazin-2-yl)phenyl)butanamide;
2-(2-(cyclopropanesulfonamido)thiazol-4-yl)-N-(6-(pyrimidin-5-yl)pyridin-3-yl)acetamide;
2-(2-(cyclopropanesulfonamido)thiazol-4-yl)-N-(5-phenylpyridin-2-yl)acetamide;
2-(2-(cyclopropanesulfonamido)thiazol-4-yl)-N-(4′-fluoro-[1,1′-biphenyl]-4-yl)acetamide;
2-(2-(cyclopropanesulfonamido)thiazol-4-yl)-N-methyl-N-(4-(pyridin-3-yl)phenyl)acetamide;
N-([2,3′-bipyridin]-5-yl)-2-(2-(cyclopropanesulfonamido)thiazol-4-yl)propanamide;
2-(2-(cyclopropanesulfonamido)thiazol-4-yl)-N-(3′-methoxy-[1,1′-biphenyl]-4-yl)-2-methylpropanamide;
2-(2-(cyclopropanesulfonamido)thiazol-4-yl)-2-methyl-N-(4-(pyridin-3-yl)phenyl)propanamide;
2-(2-(cyclopropanesulfonamido)thiazol-4-yl)-2-methyl-N-(4-(5-methylpyridin-3-yl)phenyl)propanamide;
2-(2-(cyclopropanesulfonamido)thiazol-4-yl)-2-methyl-N-(4-(pyridazin-4-yl)phenyl)propanamide;
2-(2-(cyclopropanesulfonamido)thiazol-4-yl)-2-methyl-N-(4-(pyrazin-2-yl)phenyl)propanamide;
2-(2-(cyclopropanesulfonamido)thiazol-4-yl)-N-(4-(6-methoxypyrazin-2-yl)phenyl)butanamide;
N-(3-cyano-4-(pyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(5-(6-(trifluoromethyl)pyrazin-2-yl)pyridin-2-yl)propanamide;
2-(2-(cyclopropanesulfonamido)thiazol-4-yl)-N-(2,3-difluoro-4-(pyridin-3-yl)phenyl)-2-methylpropanamide;
2-(2-(cyclopropanesulfonamido)thiazol-4-yl)-2-methyl-N-(5-(pyridin-3-yl)pyrimidin-2-yl)propanamide;
2-(2-(cyclopropanesulfonamido)thiazol-4-yl)-2-methyl-N-(5-(6-propoxypyrazin-2-yl)pyridin-2-yl)propanamide;
2-(2-(cyclopropanesulfonamido)thiazol-4-yl)-N-(3-fluoro-4-(pyrazin-2-yl)phenyl)-2-methylpropanamide;
2-(2-(cyclopropanesulfonamido)thiazol-4-yl)-2-methyl-N-(4-(pyridin-3-yl)-2-(trifluoromethoxy)phenyl)propanamide;
N-(2-chloro-4-(pyridin-3-yl)phenyl)-2-(2-(cyclopropanesulfonamido)thiazol-4-yl)-2-methylpropanamide;
2-(2-(cyclopropanesulfonamido)thiazol-4-yl)-N-(2-fluoro-4-(pyridin-3-yl)phenyl)-2-methylpropanamide;
2-(2-(cyclopropanesulfonamido)thiazol-4-yl)-N-(3-methoxy-4-(pyrazin-2-yl)phenyl)-2-methylpropanamide;
2-(2-(cyclopropanesulfonamido)thiazol-4-yl)-N-(4-(2-methoxypyridin-3-yl)phenyl)-2-methylpropanamide;
2-(2-(cyclopropanesulfonamido)thiazol-4-yl)-N-(4-(5-(hydroxymethyl)pyridin-3-yl)phenyl)acetamide;
2-(2-(cyclopropanesulfonamido)thiazol-4-yl)-N-(4-(6-methoxypyridin-3-yl)phenyl)-2-methylpropanamide;
N-(4-(5-cyanopyridin-3-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(4-(5-(trifluoromethyl)pyridin-3-yl)phenyl)propanamide;
2-(2-(cyclopropanesulfonamido)thiazol-4-yl)-2-methyl-N-(4-(5-(methylsulfonyl)pyridin-3-yl)phenyl)propanamide;
2-(2-(cyclopropanesulfonamido)thiazol-4-yl)-N-(2-methoxy-4-(5-methoxypyridin-3-yl)phenyl)-2-methylpropanamide;
2-(2-(cyclopropanesulfonamido)thiazol-4-yl)-2-methyl-N-(5′-(trifluoromethyl)-[3,3′-bipyridin]-6-yl)propanamide;
2-(2-(cyclopropanesulfonamido)thiazol-4-yl)-N-(4-(6-ethoxypyrazin-2-yl)phenyl)-2-methylpropanamide;
2-(2-(cyclopropanesulfonamido)thiazol-4-yl)-2-methyl-N-(4-(6-morpholinopyrazin-2-yl)phenyl)propanamide;
N-(4-(6-cyclobutoxypyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(4-(6-propoxypyrazin-2-yl)phenyl)propanamide;
2-(2-(cyclopropanesulfonamido)thiazol-4-yl)-N-(2-fluoro-4-(5-methoxypyridin-3-yl)phenyl)-2-methylpropanamide;
2-(2-(cyclopropanesulfonamido)thiazol-4-yl)-2-methoxy-N-(4-(6-methoxypyrazin-2-yl)phenyl)acetamide;
N-(4-(5-chloropyridin-3-yl)phenyl)-2-(2-(cyclopropanesulfonamido)thiazol-4-yl)-2-isopropoxyacetamide;
2-(2-(cyclopropanesulfonamido)thiazol-4-yl)-4-methoxy-N-(5-(6-(trifluoromethyl)pyrazin-2-yl)pyridin-2-yl)butanamide;
N-([1,1′-biphenyl]-4-yl)-2-(2-(cyclopropanesulfonamido)thiazol-4-yl)-2,2-difluoroacetamide;
2-(2-(cyclobutanesulfonamido)thiazol-4-yl)-N-(4-(6-methoxypyrazin-2-yl)phenyl)acetamide;
N-([3,3′-bipyridin]-6-yl)-2-(2-(cyclopropanesulfonamido)thiazol-4-yl)butanamide;
2-(2-(cyclopropanesulfonamido)thiazol-4-yl)-2-methyl-N-(5-phenylpyridin-2-yl)propanamide;
2-(2-(cyclopropanesulfonamido)thiazol-4-yl)-N-(5-(pyrimidin-5-yl)pyridin-2-yl)acetamide;
N-([3,3′-bipyridin]-6-yl)-2-(2-(cyclopropanesulfonamido)thiazol-4-yl)acetamide;
2-(2-(cyclopropanesulfonamido)thiazol-4-yl)-N-(6-phenylpyridin-3-yl)acetamide;
N-([2,3′-bipyridin]-5-yl)-2-(2-(cyclopropanesulfonamido)thiazol-4-yl)acetamide;
2-(2-(cyclopropanesulfonamido)thiazol-4-yl)-2-methyl-N-(4-(pyridazin-3-yl)phenyl)propanamide;
2-(2-(cyclopropanesulfonamido)thiazol-4-yl)-N-(4-(pyridazin-4-yl)phenyl)butanamide;
2-(2-(cyclopropanesulfonamido)thiazol-4-yl)-N-(4-(pyrazin-2-yl)phenyl)butanamide;
N-(4-(5-chloropyridin-3-yl)phenyl)-2-(2-(cyclopropanesulfonamido)thiazol-4-yl)-4-methoxybutanamide;
2-(2-(cyclopropanesulfonamido)thiazol-4-yl)-N-(4-(5-fluoropyridin-3-yl)phenyl)butanamide;
2-(2-(cyclopropanesulfonamido)thiazol-4-yl)-N-(4-(6-(trifluoromethyl)pyrazin-2-yl)phenyl)butanamide;
2-(2-(cyclopropanesulfonamido)thiazol-4-yl)-3-methyl-N-(4-(pyrazin-2-yl)phenyl)butanamide;
2-(2-(cyclopropanesulfonamido)thiazol-4-yl)-N-(4-(6-propoxypyrazin-2-yl)phenyl)butanamide;
2-(2-(cyclopropanesulfonamido)thiazol-4-yl)-N-(4-(6-isopropoxypyrazin-2-yl)phenyl)butanamide;
2-(2-(cyclopropanesulfonamido)thiazol-4-yl)-N-(4-(6-cyclopropoxypyrazin-2-yl)phenyl)butanamide;
N-(4-(6-chloropyrazin-2-yl)phenyl)-2-(2-(cyclopropanesulfonamido)thiazol-4-yl)butanamide;
N-(4-(6-cyanopyrazin-2-yl)phenyl)-2-(2-(cyclopropanesulfonamido)thiazol-4-yl)butanamide;
2-(2-(cyclopropanesulfonamido)thiazol-4-yl)-N-(4-(6-(trifluoromethyl)pyrazin-2-yl)phenyl)butanamide;
2-(2-(cyclopropanesulfonamido)thiazol-4-yl)-N-(4-(6-methoxypyrazin-2-yl)phenyl)acetamide;
2-(2-(cyclopropanesulfonamido)thiazol-4-yl)-N-(4-(pyrazin-2-yl)phenyl)acetamide;
N-([1,1′-biphenyl]-4-yl)-2-(cyclopropanesulfonamido)-4,5,6,7-tetrahydrobenzo[d]thiazole-4-carboxamide;
2-(cyclopropanesulfonamido)-N-(4-(pyridin-3-yl)phenyl)-4,5,6,7-tetrahydrobenzo[d]thiazole-4-carboxamide;
N-([1,1′-biphenyl]-4-yl)-2-(2-(cyclopropanesulfonamido)thiazol-4-yl)butanamide;
N-([1,1′-biphenyl]-4-yl)-2-(2-(cyclopropanesulfonamido)thiazol-4-yl)-3-methylbutanamide;
N-(3′-chloro-[1,1′-biphenyl]-4-yl)-2-(2-(cyclopropanesulfonamido)thiazol-4-yl)-2-methylpropanamide;
N-(3′-cyano-[1,1′-biphenyl]-4-yl)-2-(2-(cyclopropanesulfonamido)thiazol-4-yl)-2-methylpropanamide;
2-(2-(cyclopropanesulfonamido)thiazol-4-yl)-2,2-difluoro-N-(4-(pyridin-3-yl)phenyl)acetamide;
N-(4-(5-fluoropyridin-3-yl)phenyl)-2-(2-(cyclopropanesulfonamido)thiazol-4-yl)butanamide;
2-(2-(cyclopropanesulfonamido)thiazol-4-yl)-2-ethyl-N-(4-(pyridin-3-yl)phenyl)butanamide;
N-(4-(5-cyanopyridin-3-yl)phenyl)-2-(2-(cyclopropanesulfonamido)thiazol-4-yl)-2-ethylbutanamide;
2-(2-(cyclopropanesulfonamido)thiazol-4-yl)-N-(4-(5-ethoxypyridin-3-yl)phenyl)propanamide;
N-(4-(5-chloropyridin-3-yl)phenyl)-2-(2-(cyclopropanesulfonamido)thiazol-4-yl)propanamide;
2-(2-(cyclopropanesulfonamido)thiazol-4-yl)-N-(4-(5-ethoxypyridin-3-yl)phenyl)butanamide;
N-([1,1′-biphenyl]-4-yl)-2-(2-(cyclopropanesulfonamido)thiazol-4-yl)-2-methylpropanamide;
2-(2-(cyclopropanesulfonamido)thiazol-4-yl)-N-(4-(4-methylpyridin-3-yl)phenyl)acetamide;
2-(2-(cyclopropanesulfonamido)thiazol-4-yl)-N-(4-(5-methylpyridin-3-yl)phenyl)acetamide;
2-(2-(cyclopropanesulfonamido)thiazol-4-yl)-N-(4-(2-methylpyridin-3-yl)phenyl)acetamide;
2-(2-(cyclopropanesulfonamido)thiazol-4-yl)-N-(4-(6-methylpyridin-3-yl)phenyl)acetamide;
2-(2-(cyclopropanesulfonamido)thiazol-4-yl)-N-(4-(pyridin-3-yl)phenyl)propanamide;
2-(2-(cyclopropanesulfonamido)thiazol-4-yl)-2-methyl-N-(4-(2-methylpyridin-3-yl)phenyl)propanamide;
2-(2-(cyclopropanesulfonamido)thiazol-4-yl)-2-oxo-N-(4-(pyridin-3-yl)phenyl)acetamide;
2-(2-(cyclopropanesulfonamido)thiazol-4-yl)-2-methyl-N-(4-(6-methylpyridin-3-yl)phenyl)propanamide;
2-(2-(cyclopropanesulfonamido)thiazol-4-yl)-N-(4-(6-(trifluoromethyl)pyrazin-2-yl)phenyl)butanamide;
(R)-2-(2-(cyclopropanesulfonamido)thiazol-4-yl)-2-methoxy-N-(4-(6-methoxypyrazin-2-yl)phenyl)acetamide;
(S)-2-(2-(cyclopropanesulfonamido)thiazol-4-yl)-2-methoxy-N-(4-(6-methoxypyrazin-2-yl)phenyl)acetamide;
2-(2-(cyclopropanesulfonamido)thiazol-4-yl)-2-methyl-N-(5-(6-(2,2,2-trifluoroethoxy)pyrazin-2-yl)pyridin-2-yl)propanamide;
2-(2-(cyclopropanesulfonamido)thiazol-4-yl)-N-(3-fluoro-5-(pyrazin-2-yl)pyridin-2-yl)-2-methylpropanamide;
2-(2-(cyclopropanesulfonamido)thiazol-4-yl)-N-(5-(6-ethoxypyrazin-2-yl)-3-fluoropyridin-2-yl)-2-methylpropanamide;
2-(2-(cyclopropanesulfonamido)thiazol-4-yl)-N-(3-fluoro-5-(6-(trifluoromethyl)pyrazin-2-yl)pyridin-2-yl)-2-methylpropanamide;
N-(5-(6-cyanopyrazin-2-yl)-3-fluoropyridin-2-yl)-2-(2-(cyclopropanesulfonamido)thiazol-4-yl)-2-methylpropanamide;
2-(2-(cyclopropanesulfonamido)thiazol-4-yl)-2-methyl-N-(5′-(2,2,2-trifluoroethoxy)-[3,3′-bipyridin]-6-yl)propanamide;
2-(2-(cyclopropanesulfonamido)thiazol-4-yl)-N-(5′-(difluoromethoxy)-[3,3′-bipyridin]-6-yl)-2-methylpropanamide;
N-([2,3′-bipyridin]-5-yl)-2-(2-(cyclopropanesulfonamido)thiazol-4-yl)-2-methylpropanamide;
2-(2-(cyclopropanesulfonamido)thiazol-4-yl)-2-methyl-N-(6-(pyrimidin-5-yl)pyridin-3-yl)propanamide;
2-(2-(cyclopropanesulfonamido)thiazol-4-yl)-N-(4-(5-(difluoromethoxy)pyridin-3-yl)phenyl)-2-methylpropanamide;
2-(2-(cyclopropanesulfonamido)thiazol-4-yl)-2-ethyl-N-(4-(6-methoxypyrazin-2-yl)phenyl)butanamide;
N-(4-(5-chloropyridin-3-yl)phenyl)-2-(2-(cyclopropanesulfonamido)thiazol-4-yl)-2-ethylbutanamide;
2-(2-(cyclopropanesulfonamido)thiazol-4-yl)-2-ethyl-N-(2-fluoro-4-(pyridin-3-yl)phenyl)butanamide;
2-(2-(cyclopropanesulfonamido)thiazol-4-yl)-2-ethyl-N-(4-(pyrazin-2-yl)phenyl)butanamide;
2-(2-(cyclopropanesulfonamido)thiazol-4-yl)-2-ethyl-N-(4-(6-propoxypyrazin-2-yl)phenyl)butanamide;
2-(2-(cyclopropanesulfonamido)thiazol-4-yl)-N-(2-fluoro-4-(6-(trifluoromethyl)pyrazin-2-yl)phenyl)-2-methylpropanamide;
2-(2-(cyclopropanesulfonamido)thiazol-4-yl)-N-(4-(5-ethoxypyridin-3-yl)-2-fluorophenyl)-2-methylpropanamide;
2-(2-(cyclopropanesulfonamido)thiazol-4-yl)-N-(2-fluoro-4-(5-fluoropyridin-3-yl)phenyl)-2-methylpropanamide;
2-(2-(cyclopropanesulfonamido)thiazol-4-yl)-2-methyl-N-(4-(6-(2,2,2-trifluoroethoxy)pyrazin-2-yl)phenyl)propanamide;
N-(4-(5-chloropyridin-3-yl)-2-fluorophenyl)-2-(2-(cyclopropanesulfonamido)thiazol-4-yl)-2-methylpropanamide;
N-(4-(5-cyanopyridin-3-yl)-2-fluorophenyl)-2-(2-(cyclopropanesulfonamido)thiazol-4-yl)-2-methylpropanamide;
1-(2-(cyclopropanesulfonamido)thiazol-4-yl)-N-(4-(6-methoxypyrazin-2-yl)phenyl)cyclopentane-1-carboxamide;
2-(2-(cyclopropanesulfonamido)thiazol-4-yl)-N-(2-fluoro-4-(6-(2,2,2-trifluoroethoxy)pyrazin-2-yl)phenyl)-2-methylpropanamide;
2-(2-(cyclopropanesulfonamido)thiazol-4-yl)-N-(2-fluoro-4-(5-(2,2,2-trifluoroethoxy)pyridin-3-yl)phenyl)-2-methylpropanamide;
2-(2-(cyclopropanesulfonamido)thiazol-4-yl)-N-(2-fluoro-4-(5-(trifluoromethyl)pyridin-3-yl)phenyl)-2-methylpropanamide;
2-(2-(cyclopropanesulfonamido)thiazol-4-yl)-N-(4-(6-ethoxypyrazin-2-yl)-2-fluorophenyl)-2-methylpropanamide;
2-(2-(cyclopropanesulfonamido)thiazol-4-yl)-2-methyl-N-(4-(5-(2,2,2-trifluoroethoxy)pyridin-3-yl)phenyl)propanamide;
2-(2-(cyclopropanesulfonamido)thiazol-4-yl)-N-(4-(6-ethynylpyrazin-2-yl)phenyl)-2-methylpropanamide;
2-(2-(cyclopropanesulfonamido)thiazol-4-yl)-N-(4-(6-ethoxypyrazin-2-yl)-2-methylphenyl)-2-methylpropanamide;
N-(4-(6-chloropyrazin-2-yl)-2-methylphenyl)-2-(2-(cyclopropanesulfonamido)thiazol-4-yl)-2-methylpropanamide;
2-(2-(cyclopropanesulfonamido)thiazol-4-yl)-N-(4-(5-(difluoromethoxy)pyridin-3-yl)-2-fluorophenyl)-2-methylpropanamide;
2-(2-(cyclopropanesulfonamido)thiazol-4-yl)-2-methyl-N-(5-(pyrazin-2-yl)pyridin-2-yl)propanamide;
N-(5-(6-cyclobutoxypyrazin-2-yl)pyridin-2-yl)-2-(2-(cyclopropanesulfonamido)thiazol-4-yl)-2-methylpropanamide;
2-(2-(cyclopropanesulfonamido)thiazol-4-yl)-N-(5-(6-cyclopropoxypyrazin-2-yl)pyridin-2-yl)-2-methylpropanamide;
2-(2-(cyclopropanesulfonamido)thiazol-4-yl)-N-(5-(6-isopropoxypyrazin-2-yl)pyridin-2-yl)-2-methylpropanamide;
N-([3,3′-bipyridin]-6-yl)-2-(2-(cyclopropanesulfonamido)thiazol-4-yl)-2-ethylbutanamide;
2-(2-(cyclopropanesulfonamido)thiazol-4-yl)-N-(5′-ethoxy-[3,3′-bipyridin]-6-yl)-2-ethylbutanamide;
2-(2-(cyclopropanesulfonamido)thiazol-4-yl)-2-methyl-N-(5′-propoxy-[3,3′-bipyridin]-6-yl)propanamide;
2-(2-(cyclopropanesulfonamido)thiazol-4-yl)-2-ethyl-N-(5-(6-(trifluoromethyl)pyrazin-2-yl)pyridin-2-yl)butanamide;
N-([3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(2-methoxy-4-(pyridin-3-yl)phenyl)-2-methylpropanamide;
2-(2-(cyclopropanesulfonamido)thiazol-4-yl)-N-(3-methoxy-4-(pyridin-3-yl)phenyl)-2-methylpropanamide;
2-(2-(cyclopropanesulfonamido)thiazol-4-yl)-N-(3-fluoro-4-(pyridin-3-yl)phenyl)-2-methylpropanamide;
N-(3-cyano-4-(pyridin-3-yl)phenyl)-2-(2-(cyclopropanesulfonamido)thiazol-4-yl)-2-methylpropanamide;
N-(3-chloro-4-(pyridin-3-yl)phenyl)-2-(2-(cyclopropanesulfonamido)thiazol-4-yl)-2-methylpropanamide;
N-(4-(6-cyanopyrazin-2-yl)phenyl)-2-(2-(cyclopropanesulfonamido)thiazol-4-yl)-2-methylpropanamide;
N-(4-(6-chloropyrazin-2-yl)phenyl)-2-(2-(cyclopropanesulfonamido)thiazol-4-yl)-2-methylpropanamide;
2-(2-(cyclopropanesulfonamido)thiazol-4-yl)-2-ethyl-N-(4-(5-fluoropyridin-3-yl)phenyl)butanamide;
2-(2-(cyclopropanesulfonamido)thiazol-4-yl)-2-methyl-N-(4-(5-propoxypyridin-3-yl)phenyl)propanamide;
2-(2-(cyclopropanesulfonamido)thiazol-4-yl)-N-(4-(5-isopropoxypyridin-3-yl)phenyl)-2-methylpropanamide;
2-(2-(cyclopropanesulfonamido)thiazol-4-yl)-N-(2-fluoro-4-(5-isopropoxypyridin-3-yl)phenyl)-2-methylpropanamide;
N-(4-(6-chloropyrazin-2-yl)phenyl)-2-(2-(cyclopropanesulfonamido)thiazol-4-yl)-2-ethylbutanamide;
N-(4-(6-cyanopyrazin-2-yl)phenyl)-2-(2-(cyclopropanesulfonamido)thiazol-4-yl)-2-ethylbutanamide;
2-methyl-2-(2-(methylsulfonamido)thiazol-4-yl)-N-(4-(pyridin-3-yl)phenyl)propanamide;
2-(2-(cyclopropanesulfonamido)thiazol-4-yl)-N,2-dimethyl-N-(4-(pyridin-3-yl)phenyl)propanamide;
2-(cyclopropanesulfonamido)-N-(5-(6-(trifluoromethyl)pyrazin-2-yl)pyridin-2-yl)-5,6-dihydro-4H-cyclopenta[d]thiazole-4-carboxamide;
2-(2-(cyclopropanesulfonamido)thiazol-4-yl)-4-methoxy-N-(5-(pyrazin-2-yl)pyridin-2-yl)butanamide;
2-(2-(cyclopropanesulfonamido)thiazol-4-yl)-4-methoxy-N-(5′-methoxy-[3,3′-bipyridin]-6-yl)butanamide;
2-(2-(cyclopropanesulfonamido)thiazol-4-yl)-2-isopropoxy-N-(5-(6-(trifluoromethyl)pyrazin-2-yl)pyridin-2-yl)acetamide;
2-(2-(cyclopropanesulfonamido)thiazol-4-yl)-N-(5-(6-propoxypyrazin-2-yl)pyridin-2-yl)butanamide;
2-(2-(cyclopropanesulfonamido)thiazol-4-yl)-N-(5-(6-isopropoxypyrazin-2-yl)pyridin-2-yl)butanamide;
2-(2-(cyclopropanesulfonamido)thiazol-4-yl)-N-(5-(6-(trifluoromethyl)pyrazin-2-yl)pyridin-2-yl)butanamide;
2-(2-(cyclopropanesulfonamido)thiazol-4-yl)-N-(5-(6-methoxypyrazin-2-yl)pyridin-2-yl)butanamide;
2-(2-(cyclopropanesulfonamido)thiazol-4-yl)-N-(5-(6-ethoxypyrazin-2-yl)pyridin-2-yl)butanamide;
N-(5-(6-cyanopyrazin-2-yl)pyridin-2-yl)-2-(2-(cyclopropanesulfonamido)thiazol-4-yl)butanamide;
2-(2-(cyclopropanesulfonamido)thiazol-4-yl)-N-(5′-fluoro-[3,3′-bipyridin]-6-yl)butanamide;
N-(5′-cyano-[3,3′-bipyridin]-6-yl)-2-(2-(cyclopropanesulfonamido)thiazol-4-yl)butanamide;
2-(2-(cyclopropanesulfonamido)thiazol-4-yl)-N-(5-phenylpyridin-2-yl)butanamide;
2-(2-(cyclopropanesulfonamido)thiazol-4-yl)-N-(5-(6-(2,2,2-trifluoroethoxy)pyrazin-2-yl)pyridin-2-yl)butanamide;
2-(2-(cyclopropanesulfonamido)thiazol-4-yl)-N-(5-(6-ethoxypyrazin-2-yl)-3-fluoropyridin-2-yl)butanamide;
N-(5-(6-cyanopyrazin-2-yl)-3-fluoropyridin-2-yl)-2-(2-(cyclopropanesulfonamido)thiazol-4-yl)butanamide;
2-(2-(cyclopropanesulfonamido)thiazol-4-yl)-N-(5′-(2,2,2-trifluoroethoxy)-[3,3′-bipyridin]-6-yl)butanamide;
2-(2-(cyclopropanesulfonamido)thiazol-4-yl)-N-(5′-(difluoromethoxy)-[3,3′-bipyridin]-6-yl)butanamide;
N-(5-(6-chloropyrazin-2-yl)pyridin-2-yl)-2-(2-(cyclopropanesulfonamido)thiazol-4-yl)butanamide;
2-(2-(cyclopropanesulfonamido)thiazol-4-yl)-N-(2,3-difluoro-4-(pyridin-3-yl)phenyl)acetamide;
2-(2-(cyclopropanesulfonamido)thiazol-4-yl)-N-(4-(6-ethoxypyrazin-2-yl)phenyl)butanamide (racemic);
2-(2-(cyclopropanesulfonamido)thiazol-4-yl)-4-methoxy-N-(4-(6-methoxypyrazin-2-yl)phenyl)butanamide;
2-(2-(cyclopropanesulfonamido)thiazol-4-yl)-N-(4-(5-fluoropyridin-3-yl)phenyl)-4-methoxybutanamide;
2-(2-(cyclopropanesulfonamido)thiazol-4-yl)-N-(4-(6-ethoxypyrazin-2-yl)phenyl)propanamide;
2-(2-(cyclopropanesulfonamido)thiazol-4-yl)-N-(4-(5-(trifluoromethyl)pyridin-3-yl)phenyl)butanamide;
N-(4-(5-cyanopyridin-3-yl)phenyl)-2-(2-(cyclopropanesulfonamido)thiazol-4-yl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(2-fluoro-4-(pyrazin-2-yl)phenyl)butanamide;
2-(2-(cyclopropanesulfonamido)thiazol-4-yl)-N-(2-fluoro-4-(6-(trifluoromethyl)pyrazin-2-yl)phenyl)butanamide;
2-(2-(cyclopropanesulfonamido)thiazol-4-yl)-N-(4-(6-ethoxypyrazin-2-yl)-2-fluorophenyl)butanamide;
2-(2-(cyclopropanesulfonamido)thiazol-4-yl)-N-(4-(5-ethoxypyridin-3-yl)-2-fluorophenyl)butanamide;
2-(2-(cyclopropanesulfonamido)thiazol-4-yl)-N-(2-fluoro-4-(5-fluoropyridin-3-yl)phenyl)butanamide;
2-(2-(cyclopropanesulfonamido)thiazol-4-yl)-N-(2-fluoro-4-(pyridin-3-yl)phenyl)butanamide;
N-(4-(5-cyanopyridin-3-yl)-2-fluorophenyl)-2-(2-(cyclopropanesulfonamido)thiazol-4-yl)butanamide;
N-(4-(5-chloropyridin-3-yl)-2-fluorophenyl)-2-(2-(cyclopropanesulfonamido)thiazol-4-yl)butanamide;
(R)-2-(2-(cyclopropanesulfonamido)thiazol-4-yl)-N-(4-(6-ethoxypyrazin-2-yl)phenyl)butanamide;
(S)-2-(2-(cyclopropanesulfonamido)thiazol-4-yl)-N-(4-(6-ethoxypyrazin-2-yl)phenyl)butanamide;
N-(4-(1-(5-(6-ethoxypyrazin-2-yl)indolin-1-yl)-1-oxobutan-2-yl)thiazol-2-yl)cyclopropanesulfonamide;
2-(2-(cyclopropanesulfonamido)thiazol-4-yl)-N-(2-fluoro-4-(6-(2,2,2-trifluoroethoxy)pyrazin-2-yl)phenyl)butanamide;
2-(2-(cyclopropanesulfonamido)thiazol-4-yl)-N-(2-fluoro-4-(6-methoxypyrazin-2-yl)phenyl)butanamide;
2-(2-(cyclopropanesulfonamido)thiazol-4-yl)-N-(4-(5-(difluoromethoxy)pyridin-3-yl)-2-fluorophenyl)butanamide;
2-(2-(cyclopropanesulfonamido)thiazol-4-yl)-N-(4-(5-(difluoromethoxy)pyridin-3-yl)phenyl)butanamide;
2-(2-(cyclopropanesulfonamido)thiazol-4-yl)-N-(4-(5-(2,2,2-trifluoroethoxy)pyridin-3-yl)phenyl)butanamide;
2-(2-(cyclopropanesulfonamido)thiazol-4-yl)-N-(2-fluoro-4-(5-(2,2,2-trifluoroethoxy)pyridin-3-yl)phenyl)butanamide:
2-(cyclopropanesulfonamido)-N-(4-(pyridin-3-yl)phenyl)-5,6-dihydro-4H-cyclopenta[d]thiazole-4-carboxamide;
2-(2-(cyclopropanesulfonamido)thiazol-4-yl)-2-methoxy-N-(5-(6-(trifluoromethyl)pyrazin-2-yl)pyridin-2-yl)acetamide;
2-(2-(cyclopropanesulfonamido)thiazol-4-yl)-N-(2-methoxy-4-(pyridin-3-yl)phenyl)acetamide;
2-(2-(cyclopropanesulfonamido)thiazol-4-yl)-N-(2-fluoro-4-(pyridin-3-yl)phenyl)acetamide;
N-(4-(5-cyanopyridin-3-yl)phenyl)-2-(cyclopropanesulfonamido)-5,6-dihydro-4H-cyclopenta[d]thiazole-4-carboxamide;
2-(cyclopropanesulfonamido)-N-(4-(5-fluoropyridin-3-yl)phenyl)-5,6-dihydro-4H-cyclopenta[d]thiazole-4-carboxamide;
2-(2-(cyclopropanesulfonamido)thiazol-4-yl)-2-methoxy-N-(4-(pyridin-3-yl)phenyl)acetamide;
2-(2-(cyclopropanesulfonamido)thiazol-4-yl)-N-(2-fluoro-4-(pyridin-3-yl)phenyl)-2-methoxyacetamide;
N-(2-chloro-4-(pyridin-3-yl)phenyl)-2-(2-(cyclopropanesulfonamido)thiazol-4-yl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(5′-methoxy-[3,3′-bipyridin]-6-yl)-2-methylpropanamide;
N-(5′-chloro-[3,3′-bipyridin]-6-yl)-2-(2-(cyclopropanesulfonamido)thiazol-4-yl)-2-methylpropanamide;
N-(5′-cyano-[3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(5-fluoro-[3,3′-bipyridin]-6-yl)-2-methylpropanamide;
N-(5′-cyano-5-fluoro-[3,3′-bipyridin]-6-yl)-2-(2-(cyclopropanesulfonamido)thiazol-4-yl)-2-methylpropanamide;
N-(5′-chloro-5-fluoro-[3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(5,5′-difluoro-[3,3′-bipyridin]-6-yl)-2-methylpropanamide;
N-(5-(3-chloro-5-methylphenyl)pyridin-2-yl)-2-(2-(cyclopropanesulfonamido)thiazol-4-yl)-2-methylpropanamide;
2-(2-(cyclopropanesulfonamido)thiazol-4-yl)-N-(5-(3-methoxyphenyl)pyridin-2-yl)-2-methylpropanamide;
2-(2-(cyclopropanesulfonamido)thiazol-4-yl)-N-(5-(3-fluoro-5-methoxyphenyl)pyridin-2-yl)-2-methylpropanamide;
2-(2-(cyclopropanesulfonamido)thiazol-4-yl)-N-(5-(3,5-dimethoxyphenyl)pyridin-2-yl)-2-methylpropanamide;
2-(2-(cyclopropanesulfonamido)thiazol-4-yl)-2-methyl-N-(5-(3-(trifluoromethyl)phenyl)pyridin-2-yl)propanamide;
2-(2-(cyclopropanesulfonamido)thiazol-4-yl)-2-methyl-N-(5-(3-(trifluoromethoxy)phenyl)pyridin-2-yl)propanamide;
2-(2-(cyclopropanesulfonamido)thiazol-4-yl)-N-(5-(3-(2-hydroxypropan-2-yl)phenyl)pyridin-2-yl)-2-methylpropanamide;
2-(2-(cyclopropanesulfonamido)thiazol-4-yl)-2-methyl-N-(5-(3-morpholinophenyl)pyridin-2-yl)propanamide;
2-(2-(cyclopropanesulfonamido)thiazol-4-yl)-2-methyl-N-(6-phenylpyridin-3-yl)propanamide;
2-(2-(cyclopropanesulfonamido)thiazol-4-yl)-N-(4-(2-fluoropyridin-3-yl)phenyl)-2-methylpropanamide;
2-(2-(cyclopropanesulfonamido)thiazol-4-yl)-N-(4-(5-(hydroxymethyl)pyridin-3-yl)phenyl)-2-methylpropanamide;
2-(2-(cyclopropanesulfonamido)thiazol-4-yl)-N-(4-(2-methoxypyrimidin-5-yl)phenyl)acetamide;
N-(4′-(tert-butyl)-[1,1′-biphenyl]-4-yl)-2-(2-(cyclopropanesulfonamido)thiazol-4-yl)acetamide;
2-(2-(cyclopropanesulfonamido)thiazol-4-yl)-N-(4-(pyridin-3-yl)phenyl)acetamide;
2-(2-(cyclopropanesulfonamido)thiazol-4-yl)-N-(4-(pyridin-4-yl)phenyl)acetamide;
2-(2-(cyclopropanesulfonamido)thiazol-4-yl)-N-(2′-methoxy-[1,1′-biphenyl]-4-yl)acetamide;
2-(2-(cyclopropanesulfonamido)thiazol-4-yl)-N-(4-(pyrimidin-5-yl)phenyl)acetamide;
2-(2-(cyclopropanesulfonamido)thiazol-4-yl)-2-methyl-N-(4-(2-(trifluoromethyl)pyridin-3-yl)phenyl)propanamide;
2-(2-(cyclopropanesulfonamido)thiazol-4-yl)-2-methyl-N-(5′-methyl-[3,3′-bipyridin]-6-yl)propanamide;
2-(2-(cyclopropanesulfonamido)thiazol-4-yl)-N-(4-(2-methoxy-4-methylpyridin-3-yl)phenyl)-2-methylpropanamide;
2-(2-(cyclopropanesulfonamido)thiazol-4-yl)-N-(4-(6-methoxy-5-methylpyridin-3-yl)phenyl)-2-methylpropanamide;
N-(4-(5-chloropyridin-3-yl)phenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)phenyl)-2-methylpropanamide;
2-(2-(cyclopropanesulfonamido)thiazol-4-yl)-2-methyl-N-(4-(4-methylpyridin-3-yl)phenyl)propanamide;
2-(2-(cyclopropanesulfonamido)thiazol-4-yl)-2-methyl-N-(4-(4-(trifluoromethyl)pyridin-3-yl)phenyl)propanamide;
N-(4-(5-chloropyridin-3-yl)-2-methoxyphenyl)-2-(2-(cyclopropanesulfonamido)thiazol-4-yl)-2-methylpropanamide;
2-(2-(cyclopropanesulfonamido)thiazol-4-yl)-N-(4-(5-(dimethylamino)pyridin-3-yl)phenyl)-2-methylpropanamide;
2-(2-(cyclopropanesulfonamido)thiazol-4-yl)-N-(2-methoxy-4-(5-methylpyridin-3-yl)phenyl)-2-methylpropanamide;
2-(2-(cyclopropanesulfonamido)thiazol-4-yl)-N-(2-methoxy-4-(5-(trifluoromethyl)pyridin-3-yl)phenyl)-2-methylpropanamide;
2-(2-(cyclopropanesulfonamido)thiazol-4-yl)-N-(4-(6-methoxypyridin-3-yl)phenyl)acetamide;
2-(2-(cyclopropanesulfonamido)thiazol-4-yl)-N-(5′-fluoro-[3,3′-bipyridin]-6-yl)-2-methylpropanamide;
N-(5-(6-chloropyrazin-2-yl)pyridin-2-yl)-2-(2-(cyclopropanesulfonamido)thiazol-4-yl)-2-methylpropanamide;
N-(5-(6-cyanopyrazin-2-yl)pyridin-2-yl)-2-(2-(cyclopropanesulfonamido)thiazol-4-yl)-2-methylpropanamide;
2-(2-(cyclopropanesulfonamido)thiazol-4-yl)-2-methyl-N-(5-(pyrimidin-5-yl)pyridin-2-yl)propanamide;
2-(2-(cyclopropanesulfonamido)thiazol-4-yl)-N-(4-(6-methoxypyrazin-2-yl)phenyl)-2-methylpropanamide;
2-(2-(cyclopropanesulfonamido)thiazol-4-yl)-2-methyl-N-(4-(6-methylpyrazin-2-yl)phenyl)propanamide;
2-(2-(cyclopropanesulfonamido)thiazol-4-yl)-2-methyl-N-(4-(6-(trifluoromethyl)pyrazin-2-yl)phenyl)propanamide;
N-(4-(6-chloropyridin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(6-methoxypyridin-2-yl)phenyl)-2-methylpropanamide;
2-(2-(cyclopropanesulfonamido)thiazol-4-yl)-2-methyl-N-(4-(6-(trifluoromethyl)pyridin-2-yl)phenyl)propanamide;
2-(2-(cyclopropanesulfonamido)thiazol-4-yl)-N-(4-(4-methoxypyridin-2-yl)phenyl)-2-methylpropanamide;
2-(2-(cyclopropanesulfonamido)thiazol-4-yl)-N-(4-(6-isopropoxypyrazin-2-yl)phenyl)-2-methylpropanamide;
2-(2-(cyclopropanesulfonamido)thiazol-4-yl)-N-(4-(6-cyclopropoxypyrazin-2-yl)phenyl)-2-methylpropanamide;
2-(2-(cyclopropanesulfonamido)thiazol-4-yl)-N-(2-fluoro-4-(pyrazin-2-yl)phenyl)-2-methylpropanamide;
2-(2-(cyclopropanesulfonamido)thiazol-4-yl)-N-(2-fluoro-4-(6-methoxypyrazin-2-yl)phenyl)-2-methylpropanamide;
N-(4-(6-chloro-3-methylpyrazin-2-yl)phenyl)-2-(2-(cyclopropanesulfonamido)thiazol-4-yl)-2-methylpropanamide;
N-(4-(6-chloro-5-methylpyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(4-(6-(pyrrolidin-1-yl)pyrazin-2-yl)phenyl)propanamide;
2-(2-(cyclopropanesulfonamido)thiazol-4-yl)-N-(4-(6-(2-(dimethylamino)ethoxy)pyrazin-2-yl)phenyl)-2-methylpropanamide;
2-(2-(cyclopropanesulfonamido)thiazol-4-yl)-2-methyl-N-(4-(3-methylpyrazin-2-yl)phenyl)propanamide;
N-(4-(6-acetamidopyrazin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(5,6-dimethylpyrazin-2-yl)phenyl)-2-methylpropanamide;
2-(2-(cyclopropanesulfonamido)thiazol-4-yl)-N-(4-(6-(hydroxymethyl)pyrazin-2-yl)phenyl)-2-methylpropanamide;
2-(2-(cyclopropanesulfonamido)thiazol-4-yl)-N-(4-(3,6-dimethylpyrazin-2-yl)phenyl)-2-methylpropanamide;
2-(2-(cyclopropanesulfonamido)thiazol-4-yl)-N-(4-(5-methoxypyridin-3-yl)-2-methylphenyl)-2-methylpropanamide;
N-(4-(5-cyanopyridin-3-yl)-2-methylphenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)-2-methylphenyl)-2-methylpropanamide;
N-(4-(5-chloropyridin-3-yl)-3-methylphenyl)-2-(2-(cyclopropanesulfonamido)thiazol-4-yl)-2-methylpropanamide;
N-(4-(5-cyanopyridin-3-yl)-3-ethoxyphenyl)-2-(2-(cyclopropanesulfonamido)thiazol-4-yl)-2-methylpropanamide;
2-(2-(cyclopropanesulfonamido)thiazol-4-yl)-N-(4-(5-ethoxypyridin-3-yl)phenyl)-2-methylpropanamide;
2-(2-(cyclopropanesulfonamido)thiazol-4-yl)-N-(4-(6-cyclopropylpyrazin-2-yl)phenyl)butanamide;
2-(2-(cyclopropanesulfonamido)thiazol-4-yl)-N-(5-(5-methoxypyridin-3-yl)pyrimidin-2-yl)-2-methylpropanamide;
2-(2-(cyclopropanesulfonamido)thiazol-4-yl)-N-(5-(5-fluoropyridin-3-yl)pyrimidin-2-yl)-2-methylpropanamide;
2-(2-(cyclopropanesulfonamido)thiazol-4-yl)-2-methyl-N-(5-(5-(trifluoromethyl)pyridin-3-yl)pyrimidin-2-yl)propanamide;
N-(5-(6-ethoxypyrazin-2-yl)pyridin-2-yl)-2-methyl-2-(2-((2-methylpropyl)sulfonamido)thiazol-4-yl)propanamide;
N-(4-(6-ethoxypyrazin-2-yl)phenyl)-2-methyl-2-(2-((trifluoromethyl)sulfonamido)thiazol-4-yl)propanamide;
2-methyl-2-(2-((1-methylethyl)sulfonamido)thiazol-4-yl)-N-(4-(pyridin-3-yl)phenyl)propanamide;
N-(4-(6-ethoxypyrazin-2-yl)phenyl)-2-methyl-2-(2-((1-methylethyl)sulfonamido)thiazol-4-yl)propanamide;
2-methyl-2-(2-((1-methylcyclopropane)-1-sulfonamido)thiazol-4-yl)-N-(4-(pyridin-3-yl)phenyl)propanamide;
N-(4-(5-chloropyridin-3-yl)phenyl)-2-methyl-2-(2-((1-methylcyclopropane)-1-sulfonamido)thiazol-4-yl)propanamide;
N-(4-(6-ethoxypyrazin-2-yl)phenyl)-2-methyl-2-(2-((1-methylcyclopropane)-1-sulfonamido)thiazol-4-yl)propanamide;
2-methyl-2-(2-((1-methylcyclopropane)-1-sulfonamido)thiazol-4-yl)-N-(4-(6-(trifluoromethyl)pyrazin-2-yl)phenyl)propanamide;
2-(2-((1,1-dimethylethyl)sulfonamido)thiazol-4-yl)-2-methyl-N-(4-(pyridin-3-yl)phenyl)propanamide;
2-(2-((1,1-dimethylethyl)sulfonamido)thiazol-4-yl)-N-(4-(6-ethoxypyrazin-2-yl)phenyl)-2-methylpropanamide;
2-(2-((1,1-dimethylethyl)sulfonamido)thiazol-4-yl)-2-methyl-N-(4-(6-(trifluoromethyl)pyrazin-2-yl)phenyl)propanamide;
2-(2-(cyclobutanesulfonamido)thiazol-4-yl)-2-methyl-N-(4-(pyridin-3-yl)phenyl)propanamide;
2-(2-(cyclobutanesulfonamido)thiazol-4-yl)-N-(4-(6-ethoxypyrazin-2-yl)phenyl)-2-methylpropanamide;
2-(2-(cyclobutanesulfonamido)thiazol-4-yl)-2-methyl-N-(4-(6-(trifluoromethyl)pyrazin-2-yl)phenyl)propanamide;
N-(4-(5-cyanopyridin-3-yl)phenyl)-2-(2-(cyclopropanesulfonamido)thiazol-4-yl)-N,2-dimethylpropanamide;
2-(2-(cyclopropanesulfonamido)thiazol-4-yl)-N,2-dimethyl-N-(4-(6-(trifluoromethyl)pyrazin-2-yl)phenyl)propanamide;
2-methyl-2-(2-((2-methylpropyl)sulfonamido)thiazol-4-yl)-N-(4-(pyridin-3-yl)phenyl)propanamide;
N-(4-(6-ethoxypyrazin-2-yl)phenyl)-2-methyl-2-(2-((2-methylpropyl)sulfonamido)thiazol-4-yl)propanamide;
2-methyl-2-(2-((2-methylpropyl)sulfonamido)thiazol-4-yl)-N-(4-(6-(trifluoromethyl)pyrazin-2-yl)phenyl)propanamide;
N-(4-(5-chloropyridin-3-yl)phenyl)-2-(2-(cyclopropanesulfonamido)-5-methylthiazol-4-yl)-2-methylpropanamide;
2-(2-(cyclopropanesulfonamido)thiazol-4-yl)-N-methyl-N-(4-(pyridin-3-yl)phenyl)butanamide;
N-(4-(5-cyanopyridin-3-yl)phenyl)-2-(2-(cyclopropanesulfonamido)thiazol-4-yl)-N-methylbutanamide;
2-(2-(cyclopropanesulfonamido)-5-methylthiazol-4-yl)-N-(4-(6-ethoxypyrazin-2-yl)phenyl)-2-methylpropanamide;
N-(4-(5-chloropyridin-3-yl)phenyl)-2-(2-(cyclopropanesulfonamido)thiazol-4-yl)-N,2-dimethylpropanamide;
2-(2-(cyclopropanesulfonamido)-5-methylthiazol-4-yl)-2-methyl-N-(4-(pyridin-3-yl)phenyl)propanamide;
N-(4-(5-cyanopyridin-3-yl)-2,6-dimethylphenyl)-2-(2-(cyclopropanesulfonamido)thiazol-4-yl)-2-methylpropanamide;
N-(4-(5-chloropyridin-3-yl)-2,6-dimethylphenyl)-2-(2-(cyclopropanesulfonamido)thiazol-4-yl)-2-methylpropanamide;
N-(4-(5-cyanopyridin-3-yl)-3-methylphenyl)-2-(2-(cyclopropanesulfonamido)thiazol-4-yl)-2-methylpropanamide;
2-(2-(cyclopropanesulfonamido)thiazol-4-yl)-N-(4-(pyridin-3-yl)phenyl)butanamide;
2-(2-(cyclopropanesulfonamido)thiazol-4-yl)-N-(4-(5-fluoropyridin-3-yl)phenyl)butanamide;
2-(2-(cyclopropanesulfonamido)thiazol-4-yl)-2-methoxy-N-(4-(6-methoxypyrazin-2-yl)phenyl)acetamide;
2-(2-(cyclopropanesulfonamido)thiazol-4-yl)-N-(4-(6-ethoxypyrazin-2-yl)phenyl)butanamide;
2-amino-2-(2-(cyclopropanesulfonamido)thiazol-4-yl)-N-(4-(6-ethoxypyrazin-2-yl)phenyl)acetamide;
2-acetamido-2-(2-(cyclopropanesulfonamido)thiazol-4-yl)-N-(4-(6-ethoxypyrazin-2-yl)phenyl)acetamide;
methyl(1-(2-(cyclopropanesulfonamido)thiazol-4-yl)-2-((4-(6-ethoxypyrazin-2-yl)phenyl)amino)-2-oxoethyl)carbamate;
2-(2-(cyclopropanesulfonamido)thiazol-4-yl)-2-(dimethylamino)-N-(4-(6-ethoxypyrazin-2-yl)phenyl)acetamide;
2-(2-(cyclopropanesulfonamido)thiazol-4-yl)-N-(4-(6-ethoxypyrazin-2-yl)phenyl)-4-hydroxybutanamide;
2-(2-(cyclopropanesulfonamido)thiazol-4-yl)-N-(4-(6-ethoxypyrazin-2-yl)phenyl)-2-methoxyacetamide;
(R)-2-(2-(cyclopropanesulfonamido)thiazol-4-yl)-N-(4-(6-ethoxypyrazin-2-yl)phenyl)-2-methoxyacetamide;
(S)-2-(2-(cyclopropanesulfonamido)thiazol-4-yl)-N-(4-(6-ethoxypyrazin-2-yl)phenyl)-2-methoxyacetamide;
2-(2-((2-methoxyethyl)sulfonamido)thiazol-4-yl)-2-methyl-N-(5-(6-(trifluoromethyl)pyrazin-2-yl)pyridin-2-yl)propanamide;
2-(2-(cyclopentanesulfonamido)thiazol-4-yl)-2-methyl-N-(4-(pyridin-3-yl)phenyl)propanamide;
2-(2-(cyclopentanesulfonamido)thiazol-4-yl)-2-methyl-N-(4-(6-(trifluoromethyl)pyrazin-2-yl)phenyl)propanamide;
2-(2-(cyclopentanesulfonamido)thiazol-4-yl)-N-(4-(6-ethoxypyrazin-2-yl)phenyl)-2-methylpropanamide;
2-(2-(cyclopropanesulfonamido)thiazol-4-yl)-N-(5-(6-isopropylpyrazin-2-yl)pyridin-2-yl)-2-methylpropanamide;
2-(2-(cyclopropanesulfonamido)thiazol-4-yl)-N-(5′-ethoxy-[3,3′-bipyridin]-6-yl)-2-methylpropanamide;
2-(2-(cyclopropanesulfonamido)thiazol-4-yl)-N-(5-(6-(2-hydroxypropan-2-yl)pyrazin-2-yl)pyridin-2-yl)-2-methylpropanamide;
2-(2-(cyclopropanesulfonamido)thiazol-4-yl)-N-(5-(6-(2-methoxypropan-2-yl)pyrazin-2-yl)pyridin-2-yl)-2-methylpropanamide;
2-(2-(cyclopropanesulfonamido)-5-methylthiazol-4-yl)-2-methyl-N-(5-(6-(trifluoromethyl)pyrazin-2-yl)pyridin-2-yl)propanamide;
2-(2-(cyclopropanesulfonamido)thiazol-4-yl)-N-(5-(6-ethoxypyrazin-2-yl)pyridin-2-yl)-2-methylpropanamide;
2-(2-(cyclopropanesulfonamido)-5-methylthiazol-4-yl)-N-(5-(6-ethoxypyrazin-2-yl)pyridin-2-yl)-2-methylpropanamide;
N-(4-(5-chloropyridin-3-yl)-2-(trifluoromethyl)phenyl)-2-(2-(cyclopropanesulfonamido)thiazol-4-yl)-2-methylpropanamide;
N-(4-(5-cyanopyridin-3-yl)-2-(trifluoromethyl)phenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)-2-(trifluoromethyl)phenyl)-2-methylpropanamide;
2-(2-(cyclopropanesulfonamido)thiazol-4-yl)-2-methyl-N-(2-(trifluoromethyl)-4-(6-(trifluoromethyl)pyrazin-2-yl)phenyl)propanamide;
2-(2-(cyclopropanesulfonamido)thiazol-4-yl)-N-(4-(6-ethoxypyrazin-2-yl)-2-(trifluoromethyl)phenyl)-2-methylpropanamide;
N-(4-(5-chloropyridin-3-yl)-2,6-diethylphenyl)-2-(2-(cyclopropanesulfonamido)thiazol-4-yl)-2-methylpropanamide;
N-(4-(5-cyanopyridin-3-yl)-2,6-diethylphenyl)-2-(2-(cyclopropanesulfonamido)thiazol-4-yl)-2-methylpropanamide;
2-(2-(cyclopropanesulfonamido)-5-methylthiazol-4-yl)-N-(2-fluoro-4-(5-(trifluoromethyl)pyridin-3-yl)phenyl)-2-methylpropanamide;
N-(4-(5-chloropyridin-3-yl)-2,6-difluorophenyl)-2-(2-(cyclopropanesulfonamido)thiazol-4-yl)-2-methylpropanamide;
N-(4-(5-chloropyridin-3-yl)-2-fluoro-5-methylphenyl)-2-(2-(cyclopropanesulfonamido)thiazol-4-yl)-2-methylpropanamide;
2-(2-(cyclopropanesulfonamido)thiazol-4-yl)-N-(2-fluoro-4-(6-(2-methoxypropan-2-yl)pyrazin-2-yl)phenyl)-2-methylpropanamide;
2-(2-(cyclopropanesulfonamido)-5-methylthiazol-4-yl)-N-(2-fluoro-4-(6-(trifluoromethyl)pyrazin-2-yl)phenyl)-2-methylpropanamide;
N-(4-(6-cyanopyrazin-2-yl)-2-fluorophenyl)-2-(2-(cyclopropanesulfonamido)thiazol-4-yl)-2-methylpropanamide;
2-(2-(cyclopropanesulfonamido)thiazol-4-yl)-N-(4-(6-ethylpyrazin-2-yl)-2-fluorophenyl)-2-methylpropanamide;
2-(2-(cyclopropanesulfonamido)thiazol-4-yl)-N-(2-fluoro-4-(6-isopropoxypyrazin-2-yl)phenyl)-2-methylpropanamide;
2-(2-(cyclopropanesulfonamido)-5-methylthiazol-4-yl)-N-(4-(6-ethoxypyrazin-2-yl)-2-fluorophenyl)-2-methylpropanamide;
N-(4-(5-chloropyridin-3-yl)-2-isopropylphenyl)-2-(2-(cyclopropanesulfonamido)thiazol-4-yl)-2-methylpropanamide;
N-(4-(5-cyanopyridin-3-yl)-2-isopropylphenyl)-2-(2-(cyclopropanesulfonamido)thiazol-4-yl)-2-methylpropanamide;
2-(2-(cyclopropanesulfonamido)thiazol-4-yl)-N-(2-isopropyl-4-(6-(trifluoromethyl)pyrazin-2-yl)phenyl)-2-methylpropanamide;
2-(2-(cyclopropanesulfonamido)thiazol-4-yl)-N-(4-(6-ethoxypyrazin-2-yl)-2-isopropylphenyl)-2-methylpropanamide;
N-(4-(5-chloropyridin-3-yl)-3-fluoro-2-methylphenyl)-2-(2-(cyclopropanesulfonamido)thiazol-4-yl)-2-methylpropanamide;
N-(4-(5-chloropyridin-3-yl)-5-fluoro-2-methylphenyl)-2-(2-(cyclopropanesulfonamido)thiazol-4-yl)-2-methylpropanamide;
N-(4-(5-chloropyridin-3-yl)-2,3-dimethylphenyl)-2-(2-(cyclopropanesulfonamido)thiazol-4-yl)-2-methylpropanamide;
N-(4-(5-chloropyridin-3-yl)-2,5-dimethylphenyl)-2-(2-(cyclopropanesulfonamido)thiazol-4-yl)-2-methylpropanamide;
N-(4-(5-cyanopyridin-3-yl)-3-fluoro-2-methylphenyl)-2-(2-(cyclopropanesulfonamido)thiazol-4-yl)-2-methylpropanamide;
2-(2-(cyclopropanesulfonamido)thiazol-4-yl)-2-methyl-N-(2-methyl-4-(6-(trifluoromethyl)pyrazin-2-yl)phenyl)propanamide;
N-(4-(5-chloropyridin-3-yl)-5-fluoro-2-methoxyphenyl)-2-(2-(cyclopropanesulfonamido)thiazol-4-yl)-2-methylpropanamide;
N-(4-(5-chloropyridin-3-yl)-3-(trifluoromethyl)phenyl)-2-(2-(cyclopropanesulfonamido)thiazol-4-yl)-2-methylpropanamide;
2-(2-(cyclopropanesulfonamido)thiazol-4-yl)-N-(4-(6-ethoxypyrazin-2-yl)-3-methylphenyl)-2-methylpropanamide;
N-(4-(5-chloropyridin-3-yl)-3-ethoxyphenyl)-2-(2-(cyclopropanesulfonamido)thiazol-4-yl)-2-methylpropanamide;
N-(4-(5-chloropyridin-3-yl)phenyl)-1-(2-(cyclopropanesulfonamido)thiazol-4-yl)cyclopropane-1-carboxamide;
N-(4-(5-cyanopyridin-3-yl)phenyl)-2-(2-(cyclopropanesulfonamido)-5-methylthiazol-4-yl)-2-methylpropanamide;
2-(2-(cyclopropanesulfonamido)thiazol-4-yl)-N-(4-(6-(2-methoxypropan-2-yl)pyrazin-2-yl)phenyl)-2-methylpropanamide;
2-(5-chloro-2-(cyclopropanesulfonamido)thiazol-4-yl)-N-(4-(6-ethoxypyrazin-2-yl)phenyl)-2-methylpropanamide;
2-(2-(cyclopropanesulfonamido)-5-methoxythiazol-4-yl)-N-(4-(6-ethoxypyrazin-2-yl)phenyl)-2-methylpropanamide;
N-(4-(6-(cyclopentylmethoxy)pyrazin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(6-hydroxypyrazin-2-yl)phenyl)-2-methylpropanamide;
2-(2-(ethylsulfonamido)thiazol-4-yl)-2-methyl-N-(5′-(trifluoromethyl)-[3,3′-bipyridin]-6-yl)propanamide;
2-(2-(ethylsulfonamido)thiazol-4-yl)-2-methyl-N-(5-(6-(trifluoromethyl)pyrazin-2-yl)pyridin-2-yl)propanamide;
N-(5-(6-ethoxypyrazin-2-yl)pyridin-2-yl)-2-(2-(ethylsulfonamido)thiazol-4-yl)-2-methylpropanamide;
2-(2-(ethylsulfonamido)thiazol-4-yl)-N-(2-fluoro-4-(6-isopropoxypyrazin-2-yl)phenyl)-2-methylpropanamide;
N-(4-(5-cyanopyridin-3-yl)phenyl)-2-(2-(ethylsulfonamido)thiazol-4-yl)-2-methylpropanamide;
2-(2-(ethylsulfonamido)thiazol-4-yl)-N-(4-(5-fluoropyridin-3-yl)phenyl)-2-methylpropanamide;
2-(2-(ethylsulfonamido)thiazol-4-yl)-2-methyl-N-(4-(pyridin-3-yl)phenyl)propanamide;
2-(2-(ethylsulfonamido)thiazol-4-yl)-2-methyl-N-(4-(6-(trifluoromethyl)pyrazin-2-yl)phenyl)propanamide;
2-(2-(ethylsulfonamido)thiazol-4-yl)-N-(4-(6-isopropoxypyrazin-2-yl)phenyl)-2-methylpropanamide;
2-methyl-2-(2-(methylsulfonamido)thiazol-4-yl)-N-(5′-(trifluoromethyl)-[3,3′-bipyridin]-6-yl)propanamide;
N-(5-(6-ethoxypyrazin-2-yl)pyridin-2-yl)-2-methyl-2-(2-(methylsulfonamido)thiazol-4-yl)propanamide;
N-(2-fluoro-4-(6-(trifluoromethyl)pyrazin-2-yl)phenyl)-2-methyl-2-(2-(methylsulfonamido)thiazol-4-yl)propanamide;
N-(2-fluoro-4-(6-isopropoxypyrazin-2-yl)phenyl)-2-methyl-2-(2-(methylsulfonamido)thiazol-4-yl)propanamide;
N-(4-(5-chloropyridin-3-yl)phenyl)-2-methyl-2-(2-(methylsulfonamido)thiazol-4-yl)propanamide;
2-methyl-2-(2-(methylsulfonamido)thiazol-4-yl)-N-(4-(6-(trifluoromethyl)pyrazin-2-yl)phenyl)propanamide;
N-(4-(6-isopropoxypyrazin-2-yl)phenyl)-2-methyl-2-(2-(methylsulfonamido)thiazol-4-yl)propanamide;
2-(2-((cyclopropylmethyl)sulfonamido)thiazol-4-yl)-2-methyl-N-(5-(6-(trifluoromethyl)pyrazin-2-yl)pyridin-2-yl)propanamide;
1-(2-(cyclopropanesulfonamido)thiazol-4-yl)-N-(4-(6-(trifluoromethyl)pyrazin-2-yl)phenyl)cyclopropane-1-carboxamide;
1-(2-(cyclopropanesulfonamido)thiazol-4-yl)-N-(4-(6-ethoxypyrazin-2-yl)phenyl)cyclopropane-1-carboxamide;
2-(2-(cyclopropanesulfonamido)thiazol-4-yl)-N-(2-fluoro-4-(6-(trifluoromethyl)pyrazin-2-yl)phenyl)-4-methoxybutanamide;
2-(2-(cyclopropanesulfonamido)thiazol-4-yl)-N-(2-fluoro-4-(6-isopropoxypyrazin-2-yl)phenyl)-4-methoxybutanamide;
2-(2-(cyclopropanesulfonamido)thiazol-4-yl)-N-(5-(6-isopropylpyrazin-2-yl)pyridin-2-yl)butanamide;
2-(2-(cyclopropanesulfonamido)thiazol-4-yl)-N-(5-(6-(2-methoxypropan-2-yl)pyrazin-2-yl)pyridin-2-yl)butanamide;
2-(2-(cyclopropanesulfonamido)thiazol-4-yl)-N-(2-fluoro-4-(6-(2-methoxypropan-2-yl)pyrazin-2-yl)phenyl)butanamide;
N-(4-(6-cyanopyrazin-2-yl)-2-fluorophenyl)-2-(2-(cyclopropanesulfonamido)thiazol-4-yl)butanamide;
2-(2-(cyclopropanesulfonamido)thiazol-4-yl)-N-(4-(6-ethylpyrazin-2-yl)-2-fluorophenyl)butanamide;
2-(2-(cyclopropanesulfonamido)thiazol-4-yl)-N-(4-(6-(2-methoxypropan-2-yl)pyrazin-2-yl)phenyl)butanamide;
tert-butyl-(1-(2-(cyclopropanesulfonamido)thiazol-4-yl)-2-((4-(6-ethoxypyrazin-2-yl)phenyl)amino)-2-oxoethyl)carbamate;
2-(2-(cyclopropanesulfonamido)thiazol-4-yl)-N-(2-fluoro-4-(6-(trifluoromethyl)pyrazin-2-yl)phenyl)-2-methoxyacetamide:
2-(2-(cyclopropanesulfonamido)thiazol-4-yl)-N-(4-(6-ethoxypyrazin-2-yl)-2-fluorophenyl)-2-methoxyacetamide;
2-(2-(cyclopropanesulfonamido)thiazol-4-yl)-N-(4-(6-ethoxypyrazin-2-yl)phenyl)-2-methoxyacetamide;
2-(2-(cyclopropanesulfonamido)thiazol-4-yl)-N-(4-(6-isopropoxypyrazin-2-yl)phenyl)-2-methoxyacetamide;
2-(2-(cyclopropanesulfonamido)thiazol-4-yl)-N-(4-(6-ethoxypyrazin-2-yl)-2-fluorophenyl)butanamide;
(R)-2-(2-(cyclopropanesulfonamido)thiazol-4-yl)-N-(4-(6-ethoxypyrazin-2-yl)-2-fluorophenyl)butanamide;
(S)-2-(2-(cyclopropanesulfonamido)thiazol-4-yl)-N-(4-(6-ethoxypyrazin-2-yl)-2-fluorophenyl)butanamide;
2-(2-(cyclopropanesulfonamido)thiazol-4-yl)-N-(5-(6-(trifluoromethyl)pyrazin-2-yl)pyridin-2-yl)butanamide;
2-(2-(cyclopropanesulfonamido)thiazol-4-yl)-N-(2-fluoro-4-(5-(trifluoromethyl)pyridin-3-yl)phenyl)butanamide;
(R)-2-(2-(cyclopropanesulfonamido)thiazol-4-yl)-N-(2-fluoro-4-(5-(trifluoromethyl)pyridin-3-yl)phenyl)butanamide;
(S)-2-(2-(cyclopropanesulfonamido)thiazol-4-yl)-N-(2-fluoro-4-(5-(trifluoromethyl)pyridin-3-yl)phenyl)butanamide;
2-Amino-2-(2-(cyclopropanesulfonamido)thiazol-4-yl)-N-(5-(6-(trifluoromethyl)pyrazin-2-yl)pyridin-2-yl)acetamide hydrochloride;
2-Amino-2-(2-(cyclopropanesulfonamido)thiazol-4-yl)-N-(2-fluoro-4-(6-(trifluoromethyl)pyrazin-2-yl)phenyl)acetamide;
2-Amino-2-(2-(cyclopropanesulfonamido)thiazol-4-yl)-N-(4-(6-ethoxypyrazin-2-yl)-2-fluorophenyl)acetamide hydrochloride;
2-(2-(Cyclopropanesulfonamido)thiazol-4-yl)-2-(dimethylamino)-N-(2-fluoro-4-(6-(trifluoromethyl)pyrazin-2-yl)phenyl)acetamide;
2-(2-(Cyclopropanesulfonamido)thiazol-4-yl)-2-(dimethylamino)-N-(4-(6-ethoxypyrazin-2-yl)-2-fluorophenyl)acetamide;
2-(2-(cyclopropanesulfonamido)thiazol-4-yl)-N-(5-(6-ethoxypyrazin-2-yl)pyridin-2-yl)-2,2-difluoroacetamide;
2-(2-(cyclopropanesulfonamido)thiazol-4-yl)-N-(4-(6-ethoxypyrazin-2-yl)-2-fluorophenyl)acetamide;
2-(2-(cyclopropanesulfonamido)thiazol-4-yl)-N-(4-(6-ethoxypyrazin-2-yl)phenyl)acetamide;
2-methyl-2-(2-(methylsulfonamido)thiazol-4-yl)-N-(5-(6-(trifluoromethyl)pyrazin-2-yl)pyridin-2-yl)propanamide;
N-(2-fluoro-4-(5-(trifluoromethyl)pyridin-3-yl)phenyl)-2-methyl-2-(2-(methylsulfonamido)thiazol-4-yl)propanamide;
2-(2-((cyclopropylmethyl)sulfonamido)thiazol-4-yl)-N-(2-fluoro-4-(5-(trifluoromethyl)pyridin-3-yl)phenyl)-2-methylpropanamide;
N-(4-(5-chloro-4-methylpyridin-3-yl)phenyl)-2-(2-(cyclopropanesulfonamido)thiazol-4-yl)-2-methylpropanamide;
N-(4-(6-ethoxypyrazin-2-yl)-2-(trifluoromethyl)phenyl)-2-methyl-2-(2-(methylsulfonamido)thiazol-4-yl)propanamide;
2-(2-((cyclopropylmethyl)sulfonamido)thiazol-4-yl)-N-(2-fluoro-4-(6-(trifluoromethyl)pyrazin-2-yl)phenyl)-2-methylpropanamide;
N-(4-(6-ethoxypyrazin-2-yl)-2-fluorophenyl)-2-methyl-2-(2-(methylsulfonamido)thiazol-4-yl)propanamide;
N-(4-(6-ethoxypyrazin-2-yl)-2-fluorophenyl)-2-(2-((2-methoxyethyl)sulfonamido)thiazol-4-yl)-2-methylpropanamide;
2-(2-((cyclopropylmethyl)sulfonamido)thiazol-4-yl)-N-(4-(6-ethoxypyrazin-2-yl)-2-fluorophenyl)-2-methylpropanamide;
N-(4-(6-ethoxypyrazin-2-yl)phenyl)-2-methyl-2-(2-(methylsulfonamido)thiazol-4-yl)propanamide;
2-(2-(cyclopropanesulfonamido)thiazol-4-yl)-N-(5-(6-ethoxypyrazin-2-yl)-3-fluoropyridin-2-yl)-4-methoxybutanamide;
2-(2-(cyclopropanesulfonamido)thiazol-4-yl)-N-(5-(6-ethoxypyrazin-2-yl)pyridin-2-yl)-4-methoxybutanamide;
2-(2-(cyclopropanesulfonamido)thiazol-4-yl)-4-methoxy-N-(4-(6-(trifluoromethyl)pyrazin-2-yl)phenyl)butanamide;
2-(2-(cyclopropanesulfonamido)thiazol-4-yl)-N-(5-(6-ethoxypyrazin-2-yl)-3-methylpyridin-2-yl)butanamide;
N-(2-chloro-4-(6-ethoxypyrazin-2-yl)phenyl)-2-(2-(cyclopropanesulfonamido)thiazol-4-yl)butanamide;
N-(2-cyano-4-(6-ethoxypyrazin-2-yl)phenyl)-2-(2-(cyclopropanesulfonamido)thiazol-4-yl)butanamide;
2-(2-(cyclopropanesulfonamido)thiazol-4-yl)-N-(4-(6-ethoxypyrazin-2-yl)-2-methylphenyl)butanamide;
2-(2-(cyclopropanesulfonamido)thiazol-4-yl)-N-(4-(6-ethoxypyrazin-2-yl)-2-(trifluoromethoxy)phenyl)butanamide;
2-(2-(cyclopropanesulfonamido)thiazol-4-yl)-N-(4-(6-ethoxypyrazin-2-yl)-2-methoxyphenyl)butanamide;
2-(2-(Cyclopropanesulfonamido)thiazol-4-yl)-N-(4-(6-(ethylamino)pyrazin-2-yl)phenyl)butanamide:
2-(2-(cyclopropanesulfonamido)thiazol-4-yl)-N-(5-(6-ethoxypyrazin-2-yl)-3-fluoropyridin-2-yl)-2-methoxyacetamide;
2-(2-(cyclopropanesulfonamido)thiazol-4-yl)-N-(5-(6-ethoxypyrazin-2-yl) pyridin-2-yl)-2-methoxyacetamide;
2-(2-(cyclopropanesulfonamido)thiazol-4-yl)-N-(4-(5-fluoropyridin-3-yl)-2-(trifluoromethyl)phenyl)-2-methoxyacetamide;
2-(2-(cyclopropanesulfonamido)thiazol-4-yl)-N-(2-fluoro-4-(5-(trifluoromethyl)pyridin-3-yl)phenyl)-2-methoxyacetamide;
2-(2-(cyclopropanesulfonamido)thiazol-4-yl)-N-(4-(6-ethoxypyrazin-2-yl)-2-(trifluoromethyl)phenyl)-2-methoxyacetamide;
N-(2-chloro-4-(6-ethoxypyrazin-2-yl)phenyl)-2-(2-(cyclopropanesulfonamido)thiazol-4-yl)-2-methoxyacetamide;
N-(2-cyano-4-(6-ethoxypyrazin-2-yl)phenyl)-2-(2-(cyclopropanesulfonamido)thiazol-4-yl)-2-methoxyacetamide;
N-(2-fluoro-4-(6-(trifluoromethyl)pyrazin-2-yl)phenyl)-2-methoxy-2-(2-(methylsulfonamido)thiazol-4-yl)acetamide;
2-(2-(cyclopropanesulfonamido)thiazol-4-yl)-N-(4-(6-ethoxypyrazin-2-yl)-2,6-difluorophenyl)-2-methoxyacetamide;
2-(2-(cyclopropanesulfonamido)thiazol-4-yl)-N-(4-(6-ethoxypyrazin-2-yl)-2-(trifluoromethoxy)phenyl)-2-methoxyacetamide;
N-(4-(6-ethoxypyrazin-2-yl)phenyl)-2-methoxy-2-(2-(methylsulfonamido)thiazol-4-yl)acetamide;
2-(2-(cyclopropanesulfonamido)thiazol-4-yl)-N-(5-(6-ethoxypyrazin-2-yl)-3-fluoropyridin-2-yl)butanamide (R enantiomer);
2-(2-(cyclopropanesulfonamido)thiazol-4-yl)-N-(5-(6-ethoxypyrazin-2-yl)-3-fluoropyridin-2-yl)butanamide (S enantiomer);
2-(2-(cyclopropanesulfonamido)thiazol-4-yl)-N-(2-fluoro-4-(6-(trifluoromethyl)pyrazin-2-yl)phenyl)-2-methoxyacetamide (R enantiomer);
2-(2-(cyclopropanesulfonamido)thiazol-4-yl)-N-(2-fluoro-4-(6-(trifluoromethyl)pyrazin-2-yl)phenyl)-2-methoxyacetamide (S enantiomer);
4-(2-(cyclopropanesulfonamido)thiazol-4-yl)-N-(4-(6-ethoxypyrazin-2-yl)-2-fluorophenyl)tetrahydro-2H-pyran-4-carboxamide;
4-(2-(cyclopropanesulfonamido)thiazol-4-yl)-N-(5-(6-(trifluoromethyl)pyrazin-2-yl)pyridin-2-yl)tetrahydro-2H-pyran-4-carboxamide;
4-(2-(cyclopropanesulfonamido)thiazol-4-yl)-N-(5-(6-ethoxypyrazin-2-yl)pyridin-2-yl)tetrahydro-2H-pyran-4-carboxamide;
N-(4-(1-(4-(5-methoxypyridin-3-yl)phenyl)-2-oxopyrrolidin-3-yl)thiazol-2-yl)cyclopropanesulfonamide;
2-(2-(cyclopropanesulfonamido)thiazol-4-yl)-2-methyl-N-(5-(6-methylpyrazin-2-yl) pyridin-2-yl)propanamide; and
N-(4-(6-cyanopyrazin-2-yl)-2-methylphenyl)-2-(2-(cyclopropanesulfonamido)thiazol-4-yl)-2-methylpropanamide;
   or a pharmaceutically acceptable salt and/or pharmaceutically acceptable solvate thereof.

Such CTPS1 inhibitors are disclosed in PCT publication number WO2019106156, which is incorporated by reference in its entirety for the purpose of the CTPS1 inhibitors disclosed therein. In particular a CTPS1 inhibitor may be a compound described in any one of clauses 1 to 118 of WO2019106156, or a pharmaceutically acceptable salt and/or pharmaceutically acceptable solvate thereof, in particular a compound T1 to T465 or a pharmaceutically acceptable salt and/or pharmaceutically acceptable solvate thereof.

Alternatively, the CTPS1 inhibitor is a compound formula (III):

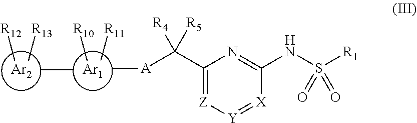

wherein
A is an amide linker having the following structure: —C(═O)NH— or —NHC(═O)—;
X is N or CH;
Y is N or CR₂;
Z is N or CR₃,
   with the proviso that when at least one of X or Z is N, Y cannot be N;
R₁ is C₁₋₅alkyl, C₀₋₂alkyleneC₃₋₅cycloalkyl which cycloalkyl is optionally substituted by CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl; and
   when A is —NHC(═O)—;
   R₄ and R₅ may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—;
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
   or a pharmaceutically acceptable salt and/or pharmaceutically acceptable solvate thereof.

More suitably, the CTPS1 inhibitor is selected from the following (‘List C’) compounds:
N-(4-(5-chloropyridin-3-yl)phenyl)-2-(2-(cyclopropanesulfonamido)pyrimidin-4-yl)butanamide;
1-(2-(cyclopropanesulfonamido)pyrimidin-4-yl)-N-(4-(6-ethoxypyrazin-2-yl)phenyl)cyclopentanecarboxamide;
2-(2-(cyclopropanesulfonamido)pyrimidin-4-yl)-N-(4-(6-methoxypyrazin-2-yl)phenyl)-2-methylpropanamide;
2-(2-(cyclopropanesulfonamido)pyrimidin-4-yl)-2-methyl-N-(4-(5-(trifluoromethyl)pyridin-3-yl)phenyl)propanamide;
2-methyl-N-(2-methyl-4-(6-methylpyrazin-2-yl)phenyl)-2-(2-(methylsulfonamido)pyrimidin-4-yl)propanamide;
2-(2-(cyclopropanesulfonamido)pyrimidin-4-yl)-N-(2-fluoro-4-(pyrazin-2-yl)phenyl)butanamide;
2-(2-(cyclopropanesulfonamido)pyrimidin-4-yl)-N-(4-(5-(trifluoromethyl)pyridin-3-yl)phenyl)butanamide;
2-(2-(cyclopropanesulfonamido)pyrimidin-4-yl)-N-(4-(6-(trifluoromethyl)pyrazin-2-yl)phenyl)acetamide;
2-(2-(cyclopropanesulfonamido)pyrimidin-4-yl)-N-(5-(6-isopropoxypyrazin-2-yl)pyridin-2-yl)-2-methylpropanamide;
2-(2-(cyclopropanesulfonamido)pyrimidin-4-yl)-N-(4-(6-ethoxypyrazin-2-yl)phenyl)-2-ethylbutanamide;
2-(2-(cyclopropanesulfonamido)pyrimidin-4-yl)-N-(2-fluoro-4-(6-(trifluoromethyl)pyrazin-2-yl)phenyl)acetamide;
2-(2-(cyclopropanesulfonamido)pyrimidin-4-yl)-N-(2-fluoro-4-(6-isopropoxypyrazin-2-yl)phenyl)acetamide;
2-(2-(cyclopropanesulfonamido)pyrimidin-4-yl)-N-(4-(5-(trifluoromethyl)pyridin-3-yl)phenyl)acetamide;
2-(2-(cyclopropanesulfonamido)pyrimidin-4-yl)-N-(4-(5-(2,2,2-trifluoroethoxy)pyridin-3-yl)phenyl)acetamide;
2-(2-(cyclopropanesulfonamido)-5-fluoropyrimidin-4-yl)-N-(4-(pyridin-3-yl)phenyl)acetamide;
2-(2-(cyclopropanesulfonamido)pyrimidin-4-yl)-N-(4-(pyridin-3-yl)phenyl)acetamide;
N-([1,1′-biphenyl]-4-yl)-2-(2-(cyclopropanesulfonamido)pyrimidin-4-yl)acetamide;
2-(2-(cyclopropanesulfonamido)pyrimidin-4-yl)-N-(4-(6-ethoxypyrazin-2-yl)phenyl)acetamide;
2-(2-(cyclopropanesulfonamido)pyrimidin-4-yl)-N-(4-(6-methoxypyrazin-2-yl)phenyl)acetamide;
2-(2-(cyclopropanesulfonamido)pyrimidin-4-yl)-N-(4-(6-(2,2,2-trifluoroethoxy)pyrazin-2-yl)phenyl)acetamide;
2-(2-(cyclopropanesulfonamido)pyrimidin-4-yl)-N-(4-(6-isopropoxypyrazin-2-yl)phenyl)acetamide;
2-(2-(cyclobutanesulfonamido)pyrimidin-4-yl)-N-(5-(6-ethoxypyrazin-2-yl)pyridin-2-yl)-2-methylpropanamide;
2-(2-(cyclobutanesulfonamido)pyrimidin-4-yl)-N-(2-fluoro-4-(6-isopropoxypyrazin-2-yl)phenyl)-2-methylpropanamide;
2-(2-(cyclobutanesulfonamido)pyrimidin-4-yl)-N-(4-(6-ethoxypyrazin-2-yl)-2-methylphenyl)-2-methylpropanamide;
2-(2-(cyclobutanesulfonamido)pyrimidin-4-yl)-N-(4-(6-methoxypyrazin-2-yl)phenyl)-2-methylpropanamide;
2-(2-(cyclobutanesulfonamido)pyrimidin-4-yl)-N-(4-(6-ethoxypyrazin-2-yl)phenyl)-2-methylpropanamide;
2-(2-(cyclopropanesulfonamido)pyrimidin-4-yl)-N-(5-(6-ethoxypyrazin-2-yl)-3-fluoropyridin-2-yl)-2-methylpropanamide;
2-(2-(cyclopropanesulfonamido)pyrimidin-4-yl)-N-(5′-ethoxy-[3,3′-bipyridin]-6-yl)-2-methylpropanamide;
N-([3,3′-bipyridin]-6-yl)-2-(2-(cyclopropanesulfonamido)pyrimidin-4-yl)-2-methylpropanamide;
2-(2-(cyclopropanesulfonamido)pyrimidin-4-yl)-2-methyl-N-(5-(6-(trifluoromethyl)pyrazin-2-yl)pyridin-2-yl)propanamide;
2-(2-(cyclopropanesulfonamido)pyrimidin-4-yl)-N-(5-(6-ethoxypyrazin-2-yl)pyridin-2-yl)-2-methylpropanamide;
2-(2-(cyclopropanesulfonamido)pyrimidin-4-yl)-N-(5-(6-cyclopropoxypyrazin-2-yl)pyridin-2-yl)-2-methylpropanamide;
N-(2-chloro-4-(6-ethoxypyrazin-2-yl)phenyl)-2-(2-(cyclopropanesulfonamido)pyrimidin-4-yl)-2-methylpropanamide;
N-(2-cyano-4-(6-ethoxypyrazin-2-yl)phenyl)-2-(2-(cyclopropanesulfonamido)pyrimidin-4-yl)-2-methylpropanamide;
2-(2-(cyclopropanesulfonamido)pyrimidin-4-yl)-N-(2-fluoro-4-(5-isopropoxypyridin-3-yl)phenyl)-2-methylpropanamide;
2-(2-(cyclopropanesulfonamido)pyrimidin-4-yl)-N-(2-fluoro-4-(pyridin-3-yl)phenyl)-2-methylpropanamide;
2-(2-(cyclopropanesulfonamido)pyrimidin-4-yl)-N-(2-fluoro-4-(6-(trifluoromethyl)pyrazin-2-yl)phenyl)-2-methylpropanamide;
2-(2-(cyclopropanesulfonamido)pyrimidin-4-yl)-N-(4-(6-ethoxypyrazin-2-yl)-2-fluorophenyl)-2-methylpropanamide;
2-(2-(cyclopropanesulfonamido)pyrimidin-4-yl)-N-(2-fluoro-4-(6-isopropoxypyrazin-2-yl)phenyl)-2-methylpropanamide;
2-(2-(cyclopropanesulfonamido)pyrimidin-4-yl)-N-(4-(6-ethoxypyrazin-2-yl)-2-fluoro-5-methylphenyl)-2-methylpropanamide;
2-(2-(cyclopropanesulfonamido)pyrimidin-4-yl)-N-(4-(6-ethoxypyrazin-2-yl)-2,6-difluorophenyl)-2-methylpropanamide;
2-(2-(cyclopropanesulfonamido)pyrimidin-4-yl)-N-(2-fluoro-4-(pyrazin-2-yl)phenyl)-2-methylpropanamide;
2-(2-(cyclopropanesulfonamido)pyrimidin-4-yl)-2-methyl-N-(2-methyl-4-(6-(trifluoromethyl)pyrazin-2-yl)phenyl)propanamide;
2-(2-(cyclopropanesulfonamido)pyrimidin-4-yl)-N-(4-(6-ethoxypyrazin-2-yl)-2,3-dimethylphenyl)-2-methylpropanamide;
2-(2-(cyclopropanesulfonamido)pyrimidin-4-yl)-N-(4-(6-ethoxypyrazin-2-yl)-5-fluoro-2-methylphenyl)-2-methylpropanamide;
2-(2-(cyclopropanesulfonamido)pyrimidin-4-yl)-N-(4-(6-ethoxypyrazin-2-yl)-2,5-dimethylphenyl)-2-methylpropanamide;
2-(2-(cyclopropanesulfonamido)pyrimidin-4-yl)-N-(4-(6-ethoxypyrazin-2-yl)-2-(trifluoromethoxy)phenyl)-2-methylpropanamide;
2-(2-(cyclopropanesulfonamido)pyrimidin-4-yl)-N-(4-(6-ethoxypyrazin-2-yl)-5-fluoro-2-methoxyphenyl)-2-methylpropanamide;
2-(2-(cyclopropanesulfonamido)pyrimidin-4-yl)-N-(4-(6-ethoxypyrazin-2-yl)-2-methoxyphenyl)-2-methylpropanamide;
2-(2-(cyclopropanesulfonamido)pyrimidin-4-yl)-2-methyl-N-(4-(pyrimidin-5-yl)phenyl)propanamide;
N-(4-(5-chloropyridin-3-yl)phenyl)-2-(2-(cyclopropanesulfonamido)pyrimidin-4-yl)-2-methylpropanamide;
N-(4-(5-cyanopyridin-3-yl)phenyl)-2-(2-(cyclopropanesulfonamido)pyrimidin-4-yl)-2-methylpropanamide;
2-(2-(cyclopropanesulfonamido)pyrimidin-4-yl)-N-(4-(5-fluoropyridin-3-yl)phenyl)-2-methylpropanamide;
2-(2-(cyclopropanesulfonamido)pyrimidin-4-yl)-2-methyl-N-(4-(5-methylpyridin-3-yl)phenyl)propanamide;
2-(2-(cyclopropanesulfonamido)pyrimidin-4-yl)-N-(4-(5-(difluoromethoxy)pyridin-3-yl)phenyl)-2-methylpropanamide;
2-(2-(cyclopropanesulfonamido)pyrimidin-4-yl)-N-(4-(5-methoxypyridin-3-yl)phenyl)-2-methylpropanamide;
2-(2-(cyclopropanesulfonamido)pyrimidin-4-yl)-N-(4-(5-ethoxypyridin-3-yl)phenyl)-2-methylpropanamide;
2-(2-(cyclopropanesulfonamido)pyrimidin-4-yl)-N-(4-(5-isopropoxypyridin-3-yl)phenyl)-2-methylpropanamide;
2-(2-(cyclopropanesulfonamido)pyrimidin-4-yl)-2-methyl-N-(4-(pyridin-3-yl)phenyl)propanamide;
2-(2-(cyclopropanesulfonamido)pyrimidin-4-yl)-2-methyl-N-(3′-(trifluoromethyl)-[1,1′-biphenyl]-4-yl)propanamide;
N-(3′-chloro-[1,1′-biphenyl]-4-yl)-2-(2-(cyclopropanesulfonamido)pyrimidin-4-yl)-2-methylpropanamide;
N-(3′-cyano-[1,1′-biphenyl]-4-yl)-2-(2-(cyclopropanesulfonamido)pyrimidin-4-yl)-2-methylpropanamide;
2-(2-(cyclopropanesulfonamido)pyrimidin-4-yl)-N-(3′-ethoxy-[1,1′-biphenyl]-4-yl)-2-methylpropanamide;
2-(2-(cyclopropanesulfonamido)pyrimidin-4-yl)-2-methyl-N-(4-(6-(trifluoromethyl)pyrazin-2-yl)phenyl)propanamide;
2-(2-(cyclopropanesulfonamido)pyrimidin-4-yl)-N-(4-(6-ethoxypyrazin-2-yl)phenyl)-2-methylpropanamide;
2-(2-(cyclopropanesulfonamido)pyrimidin-4-yl)-N-(4-(6-cyclopropoxypyrazin-2-yl)phenyl)-2-methylpropanamide;
2-(2-(cyclopropanesulfonamido)pyrimidin-4-yl)-N-(4-(6-isopropoxypyrazin-2-yl)phenyl)-2-methylpropanamide;
2-(2-(cyclopropanesulfonamido)-5-fluoropyrimidin-4-yl)-N-(4-(6-ethoxypyrazin-2-yl)phenyl)-2-methylpropanamide;
N-(4-(6-ethoxypyrazin-2-yl)phenyl)-2-methyl-2-(2-((1-methylcyclopropane)-1-sulfonamido)pyrimidin-4-yl)propanamide;
2-(2-(cyclopropanesulfonamido)-5-methylpyrimidin-4-yl)-N-(4-(6-ethoxypyrazin-2-yl)phenyl)-2-methylpropanamide;
2-(2-(cyclopropanesulfonamido)pyrimidin-4-yl)-2-methyl-N-(4-(pyrazin-2-yl)phenyl)propanamide;
N-(4-(6-ethoxypyrazin-2-yl)-2-fluorophenyl)-2-(2-(ethylsulfonamido)pyrimidin-4-yl)-2-methylpropanamide;
2-(2-(ethylsulfonamido)pyrimidin-4-yl)-2-methyl-N-(4-(6-(trifluoromethyl)pyrazin-2-yl)phenyl)propanamide;
N-(4-(6-ethoxypyrazin-2-yl)phenyl)-2-(2-(ethylsulfonamido)pyrimidin-4-yl)-2-methylpropanamide;
N-(5-(6-ethoxypyrazin-2-yl)-3-fluoropyridin-2-yl)-2-methyl-2-(2-(methylsulfonamido)pyrimidin-4-yl)propanamide;
N-(5-(6-ethoxypyrazin-2-yl)pyridin-2-yl)-2-methyl-2-(2-(methylsulfonamido)pyrimidin-4-yl)propanamide;
N-(2-fluoro-4-(5-isopropoxypyridin-3-yl)phenyl)-2-methyl-2-(2-(methylsulfonamido)pyrimidin-4-yl)propanamide;
N-(2-fluoro-4-(6-isopropoxypyrazin-2-yl)phenyl)-2-methyl-2-(2-(methylsulfonamido)pyrimidin-4-yl)propanamide;
2-methyl-N-(2-methyl-4-(6-(trifluoromethyl)pyrazin-2-yl)phenyl)-2-(2-(methylsulfonamido)pyrimidin-4-yl)propanamide;
2-methyl-2-(2-(methylsulfonamido)pyrimidin-4-yl)-N-(4-(6-(trifluoromethyl)pyrazin-2-yl)phenyl)propanamide;
N-(4-(6-ethoxypyrazin-2-yl)phenyl)-2-methyl-2-(2-(methylsulfonamido)pyrimidin-4-yl)propanamide;
2-(2-((1,1-dimethylethyl)sulfonamido)pyrimidin-4-yl)-N-(4-(6-ethoxypyrazin-2-yl)phenyl)-2-methylpropanamide;
1-(2-(cyclopropanesulfonamido)pyrimidin-4-yl)-N-(4-(6-ethoxypyrazin-2-yl)phenyl)cyclopropanecarboxamide;
2-(2-(cyclopropanesulfonamido)pyrimidin-4-yl)-N-(5′-(trifluoromethyl)-[3,3′-bipyridin]-6-yl)butanamide;
2-(2-(cyclopropanesulfonamido)pyrimidin-4-yl)-N-(5′-(2,2,2-trifluoroethoxy)-[3,3′-bipyridin]-6-yl)butanamide;
N-([3,3′-bipyridin]-6-yl)-2-(2-(cyclopropanesulfonamido)pyrimidin-4-yl)butanamide;
2-(2-(cyclopropanesulfonamido)pyrimidin-4-yl)-N-(5-(6-(trifluoromethyl)pyrazin-2-yl)pyridin-2-yl)butanamide;
2-(2-(cyclopropanesulfonamido)pyrimidin-4-yl)-N-(5-(6-ethoxypyrazin-2-yl)pyridin-2-yl)butanamide;
2-(2-(cyclopropanesulfonamido)pyrimidin-4-yl)-N-(5-(6-isopropoxypyrazin-2-yl)pyridin-2-yl)butanamide;
N-(4-(5-chloropyridin-3-yl)-2-fluorophenyl)-2-(2-(cyclopropanesulfonamido)pyrimidin-4-yl)butanamide;
2-(2-(cyclopropanesulfonamido)pyrimidin-4-yl)-N-(2-fluoro-4-(5-(2,2,2-trifluoroethoxy)pyridin-3-yl)phenyl)butanamide;
2-(2-(cyclopropanesulfonamido)pyrimidin-4-yl)-N-(2-fluoro-4-(5-isopropoxypyridin-3-yl)phenyl)butanamide;
2-(2-(cyclopropanesulfonamido)pyrimidin-4-yl)-N-(2-fluoro-4-(pyridin-3-yl)phenyl)butanamide;
2-(2-(cyclopropanesulfonamido)pyrimidin-4-yl)-N-(2-fluoro-4-(6-(trifluoromethyl)pyrazin-2-yl)phenyl)butanamide;
2-(2-(cyclopropanesulfonamido)pyrimidin-4-yl)-N-(2-fluoro-4-(6-methoxypyrazin-2-yl)phenyl)butanamide;
2-(2-(cyclopropanesulfonamido)pyrimidin-4-yl)-N-(4-(6-ethoxypyrazin-2-yl)-2-fluorophenyl)butanamide;
2-(2-(cyclopropanesulfonamido)pyrimidin-4-yl)-N-(2-fluoro-4-(6-isopropoxypyrazin-2-yl)phenyl)butanamide;
2-(2-(cyclopropanesulfonamido)pyrimidin-4-yl)-N-(2-fluoro-4-(6-(2,2,2-trifluoroethoxy)pyrazin-2-yl)phenyl)butanamide;
N-(4-(5-cyanopyridin-3-yl)phenyl)-2-(2-(cyclopropanesulfonamido)pyrimidin-4-yl)butanamide;
2-(2-(cyclopropanesulfonamido)pyrimidin-4-yl)-N-(4-(5-(2,2,2-trifluoroethoxy)pyridin-3-yl)phenyl)butanamide;
2-(2-(cyclopropanesulfonamido)pyrimidin-4-yl)-N-(4-(5-isopropoxypyridin-3-yl)phenyl)butanamide;
2-(2-(cyclopropanesulfonamido)pyrimidin-4-yl)-N-(4-(pyridin-3-yl)phenyl)butanamide;
2-(2-(cyclopropanesulfonamido)pyrimidin-4-yl)-N-(4-(6-(trifluoromethyl)pyrazin-2-yl)phenyl)butanamide;
N-(4-(6-chloropyrazin-2-yl)phenyl)-2-(2-(cyclopropanesulfonamido)pyrimidin-4-yl)butanamide;
2-(2-(cyclopropanesulfonamido)pyrimidin-4-yl)-N-(4-(6-ethoxypyrazin-2-yl)phenyl)butanamide;
2-(2-(cyclopropanesulfonamido)pyrimidin-4-yl)-N-(4-(6-methoxypyrazin-2-yl)phenyl)butanamide;
2-(2-(cyclopropanesulfonamido)pyrimidin-4-yl)-N-(4-(6-isopropoxypyrazin-2-yl)phenyl)butanamide;
2-(2-(cyclopropanesulfonamido)pyrimidin-4-yl)-N-(4-(6-(2,2,2-trifluoroethoxy)pyrazin-2-yl)phenyl)butanamide;
2-(2-(cyclopropanesulfonamido)pyrimidin-4-yl)-N-(4-(pyrazin-2-yl)phenyl)butanamide;
2-(2-(cyclopropanesulfonamido)pyrimidin-4-yl)-N-(4-(6-ethoxypyrazin-2-yl)phenyl)-4-methoxybutanamide;
2-(2-(cyclopropanesulfonamido)pyrimidin-4-yl)-N-(4-(pyridin-3-yl)phenyl)propenamide;
2-(2-(cyclopropanesulfonamido)pyrimidin-4-yl)-N-(5-(6-ethoxypyrazin-2-yl)pyridin-2-yl)-2-(R)-fluorobutanamide;
2-(2-(cyclopropanesulfonamido)pyrimidin-4-yl)-N-(5-(6-ethoxypyrazin-2-yl)pyridin-2-yl)-2-(S)-fluorobutanamide;
2-(2-(cyclopropanesulfonamido)pyrimidin-4-yl)-N-(5-(6-ethoxypyrazin-2-yl)pyridin-2-yl)-2-fluorobutanamide;
4-(2-(cyclopropanesulfonamido)pyrimidin-4-yl)-N-(5-(6-ethoxypyrazin-2-yl)pyridin-2-yl)tetrahydro-2H-pyran-4-carboxamide;
2-(2-(cyclopropanesulfonamido)pyrimidin-4-yl)-N-(5-(6-isopropylpyrazin-2-yl)pyridin-2-yl)-2-methylpropanamide;
2-(2-(cyclopropanesulfonamido)pyrimidin-4-yl)-N-(4-(6-ethoxypyrazin-2-yl)-2-fluorophenyl)-2,2-difluoroacetamide;
N-((2-(cyclopropanesulfonamido)pyrimidin-4-yl)methyl)-4-(6-ethoxypyrazin-2-yl)benzamide;
2-(2-(cyclopropanesulfonamido)pyrimidin-4-yl)-2-methyl-N-(5-(6-(prop-1-en-2-yl)pyrazin-2-yl)pyridin-2-yl)propanamide;
2-(2-(cyclopropanesulfonamido)-6-methylpyrimidin-4-yl)-N-(5-(6-ethoxypyrazin-2-yl)pyridin-2-yl)-2-methylpropanamide;
2-(2-(cyclopropanesulfonamido)-6-(trifluoromethyl)pyrimidin-4-yl)-N-(5-(6-ethoxypyrazin-2-yl)pyridin-2-yl)-2-methylpropanamide;
2-(2-(cyclopropanesulfonamido)pyrimidin-4-yl)-N-(5-(6-cyclopropylpyrazin-2-yl)pyridin-2-yl)-2-methylpropanamide;
2-(2-(cyclopropanesulfonamido)pyrimidin-4-yl)-N-(6-(6-ethoxypyrazin-2-yl)pyridin-3-yl)-2-methylpropanamide;
2-(2-(cyclopropanesulfonamido)pyrimidin-4-yl)-N-(4-(6-cyclopropylpyrazin-2-yl)-2-fluorophenyl)-2-methylpropanamide;
2-(2-(cyclopropanesulfonamido)-6-methylpyrimidin-4-yl)-N-(4-(6-ethoxypyrazin-2-yl)-2-fluorophenyl)-2-methylpropanamide;
2-(2-(cyclopropanesulfonamido)-6-(trifluoromethyl)pyrimidin-4-yl)-N-(4-(6-ethoxypyrazin-2-yl)-2-fluorophenyl)-2-methylpropanamide;
2-(2-(cyclopropanesulfonamido)pyrimidin-4-yl)-2-methyl-N-(4-(6-(prop-1-en-2-yl)pyrazin-2-yl)phenyl)propanamide;
2-(2-(cyclopropanesulfonamido)pyrimidin-4-yl)-N-(4-(6-isopropylpyrazin-2-yl)phenyl)-2-methylpropanamide;
2-(2-(cyclopropanesulfonamido)pyrimidin-4-yl)-N-(4-(6-(dimethylamino)pyrazin-2-yl)phenyl)-2-methylpropanamide;
2-(2-(cyclopropanesulfonamido)-6-methylpyrimidin-4-yl)-N-(4-(6-ethoxypyrazin-2-yl)phenyl)-2-methylpropanamide;
2-(2-(cyclopropanesulfonamido)-6-(trifluoromethyl)pyrimidin-4-yl)-N-(4-(6-ethoxypyrazin-2-yl)phenyl)-2-methylpropanamide;
2-(2-(cyclopropanesulfonamido)-6-methoxypyrimidin-4-yl)-2-methyl-N-(4-(pyridin-3-yl)phenyl)propanamide;
1-(2-(cyclopropanesulfonamido)pyrimidin-4-yl)-N-(5-(6-ethoxypyrazin-2-yl)pyridin-2-yl)cyclopentane-1-carboxamide;
4-(2-(cyclopropanesulfonamido)pyrimidin-4-yl)-N-(4-(6-ethoxypyrazin-2-yl)phenyl)tetrahydro-2H-pyran-4-carboxamide;
N-(5-(6-ethoxypyrazin-2-yl) pyridin-2-yl)-4-(2-(methylsulfonamido)pyrimidin-4-yl)piperidine-4-carboxamide;
tert-butyl 4-(2-(cyclopropanesulfonamido)pyrimidin-4-yl)-4-((5-(6-ethoxypyrazin-2-yl)pyridin-2-yl)carbamoyl)piperidine-1-carboxylate;
4-(2-(cyclopropanesulfonamido)pyrimidin-4-yl)-N-(5-(6-ethoxypyrazin-2-yl)pyridin-2-yl)piperidine-4-carboxamide;
tert-butyl 3-(2-(cyclopropanesulfonamido)pyrimidin-4-yl)-3-((5-(6-ethoxypyrazin-2-yl)pyridin-2-yl)carbamoyl)azetidine-1-carboxylate;
tert-butyl 4-((5-(6-ethoxypyrazin-2-yl)pyridin-2-yl)carbamoyl)-4-(2-(methylsulfonamido)pyrimidin-4-yl)piperidine-1-carboxylate;
4-(2-(cyclopropanesulfonamido)pyrimidin-4-yl)-N-(4-(6-ethoxypyrazin-2-yl)-2-fluorophenyl)tetrahydro-2H-pyran-4-carboxamide;
2-(2-(cyclopropanesulfonamido)pyrimidin-4-yl)-N-(5-(6-ethoxypyrazin-2-yl)-3-fluoropyridin-2-yl)-4-methoxybutanamide;
2-(2-(cyclopropanesulfonamido)pyrimidin-4-yl)-N-(5-(6-ethoxypyrazin-2-yl)pyridin-2-yl)-4-methoxybutanamide;
2-(2-(cyclopropanesulfonamido)pyrimidin-4-yl)-N-(4-(6-ethoxypyrazin-2-yl)-2-fluorophenyl)-4-methoxybutanamide;
N-(5-(6-ethoxypyrazin-2-yl)pyridin-2-yl)-4-methoxy-2-methyl-2-(2-(methylsulfonamido)pyrimidin-4-yl)butanamide;
N-(5′-chloro-[3,3′-bipyridin]-6-yl)-2-(2-(cyclopropanesulfonamido)pyrimidin-4-yl)butanamide;
N-(5′-chloro-[3,3′-bipyridin]-6-yl)-2-(2-(cyclopropanesulfonamido)pyrimidin-4-yl)-2-fluorobutanamide;
2-(2-(cyclopropanesulfonamido)pyrimidin-4-yl)-N-(5-(6-cyclopropylpyrazin-2-yl)pyridin-2-yl)-2-fluorobutanamide;
N-(5-(6-ethoxypyrazin-2-yl)pyridin-2-yl)-2-fluoro-2-(2-(methylsulfonamido)pyrimidin-4-yl)butanamide;
2-(2-(cyclopropanesulfonamido)pyrimidin-4-yl)-N-(5-(6-ethoxypyrazin-2-yl)-3-methylpyridin-2-yl)butanamide;
2-(2-(cyclopropanesulfonamido)pyrimidin-4-yl)-N-(5-(6-cyclopropylpyrazin-2-yl)pyridin-2-yl)butanamide;
2-(2-(cyclopropanesulfonamido)pyrimidin-4-yl)-N-(5-(6-(2,2,2-trifluoroethoxy)pyrazin-2-yl)pyridin-2-yl)butanamide;
2-(2-(cyclopropanesulfonamido)pyrimidin-4-yl)-N-(3-fluoro-5-(6-methoxypyrazin-2-yl)pyridin-2-yl)butanamide;
2-(2-(cyclopropanesulfonamido)pyrimidin-4-yl)-N-(5-(6-methoxypyrazin-2-yl)pyridin-2-yl)butanamide;
2-(2-(cyclopropanesulfonamido)pyrimidin-4-yl)-N-(4-(6-cyclopropylpyrazin-2-yl)-2-fluorophenyl)butanamide;
2-(2-(cyclopropanesulfonamido)pyrimidin-4-yl)-N-(4-(6-ethoxypyrazin-2-yl)-2-methylphenyl)butanamide;
2-(2-(cyclopropanesulfonamido)pyrimidin-4-yl)-N-(5-(6-ethoxypyrazin-2-yl)-3-fluoropyridin-2-yl)butanamide;
2-(2-(cyclopropanesulfonamido)pyrimidin-4-yl)-N-(5-(6-ethoxypyrazin-2-yl)pyridin-2-yl)-2-methylbutanamide;
2-(2-(cyclopropanesulfonamido)pyrimidin-4-yl)-N-(5-(6-ethoxypyrazin-2-yl)pyridin-2-yl)-2-fluoro-3-methylbutanamide;
2-(2-(cyclopropanesulfonamido)pyrimidin-4-yl)-N-(4-(6-ethoxypyrazin-2-yl)-2-fluorophenyl)-3-methylbutanamide;
2-(2-(cyclopropanesulfonamido)pyrimidin-4-yl)-N-(5-(6-ethoxypyrazin-2-yl)pyridin-2-yl)-3-methylbutanamide;
2-(2-(cyclopropanesulfonamido)pyrimidin-4-yl)-N-(4-(6-ethoxypyrazin-2-yl)phenyl)-2-methoxyacetamide;
N-(5-(6-ethoxypyrazin-2-yl)pyridin-2-yl)-2-fluoro-2-(2-(methylsulfonamido)pyrimidin-4-yl)-(R)-butanamide;
N-(5-(6-ethoxypyrazin-2-yl)pyridin-2-yl)-2-fluoro-2-(2-(methylsulfonamido)pyrimidin-4-yl)-(S)-butanamide;
N-(4-(5-chloropyridin-3-yl)phenyl)-2-(6-(cyclopropanesulfonamido)pyridin-2-yl)acetamide;
N-(4-(5-cyanopyridin-3-yl)phenyl)-2-(6-(cyclopropanesulfonamido)pyridin-2-yl)acetamide;
2-(6-(cyclopropanesulfonamido)pyridin-2-yl)-N-(4-(5-fluoropyridin-3-yl)phenyl)acetamide;
2-(6-(cyclopropanesulfonamido)pyridin-2-yl)-N-(4-(5-methoxypyridin-3-yl)phenyl)acetamide;
2-(6-(cyclopropanesulfonamido)pyridin-2-yl)-N-(4-(pyridin-3-yl)phenyl)acetamide;
2-(6-(cyclopropanesulfonamido)pyridin-2-yl)-N-(4-(6-(trifluoromethyl)pyrazin-2-yl)phenyl)acetamide;
2-(6-(cyclopropanesulfonamido)pyridin-2-yl)-N-(4-(6-methoxypyrazin-2-yl)phenyl)acetamide;
2-(6-(cyclopropanesulfonamido)pyridin-2-yl)-N-(4-(pyrazin-2-yl)phenyl)acetamide;
N-([3,3′-bipyridin]-6-yl)-2-(6-(cyclopropanesulfonamido)pyridin-2-yl)-2-methylpropanamide;
N-(4-(5-chloropyridin-3-yl)phenyl)-2-(6-(cyclopropanesulfonamido)pyridin-2-yl)-2-methylpropanamide;
2-(6-(cyclopropanesulfonamido)pyridin-2-yl)-N-(4-(5-fluoropyridin-3-yl)phenyl)-2-methylpropanamide;
2-(6-(cyclopropanesulfonamido)pyridin-2-yl)-N-(4-(5-ethoxypyridin-3-yl)phenyl)-2-methylpropanamide;
2-(6-(cyclopropanesulfonamido)pyridin-2-yl)-2-methyl-N-(4-(pyridin-3-yl)phenyl)propanamide;
2-(6-(cyclopropanesulfonamido)pyridin-2-yl)-N-(2-fluoro-4-(pyrazin-2-yl)phenyl)-2-methylpropanamide;
2-(6-(cyclopropanesulfonamido)pyridin-2-yl)-2-methyl-N-(4-(6-(trifluoromethyl)pyrazin-2-yl)phenyl)propanamide;
N-(4-(6-chloropyrazin-2-yl)phenyl)-2-(6-(cyclopropanesulfonamido)pyridin-2-yl)-2-methylpropanamide;
2-(6-(cyclopropanesulfonamido)pyridin-2-yl)-N-(4-(6-ethoxypyrazin-2-yl)phenyl)-2-methylpropanamide;
2-(6-(cyclopropanesulfonamido)pyridin-2-yl)-N-(4-(6-methoxypyrazin-2-yl)phenyl)-2-methylpropanamide;
2-(6-(cyclopropanesulfonamido)pyridin-2-yl)-2-methyl-N-(4-(pyrazin-2-yl)phenyl)propanamide;
4-(6-(cyclopropanesulfonamido)pyridin-2-yl)-N-(5-(6-ethoxypyrazin-2-yl)pyridin-2-yl)tetrahydro-2H-pyran-4-carboxamide;
2-(6-(cyclopropanesulfonamido)pyridin-2-yl)-N-(5-(6-(trifluoromethyl)pyrazin-2-yl)pyridin-2-yl)butanamide;
2-(6-(cyclopropanesulfonamido)pyridin-2-yl)-N-(5-(6-ethoxypyrazin-2-yl)pyridin-2-yl)butanamide;
N-(4-(5-chloropyridin-3-yl)phenyl)-2-(6-(cyclopropanesulfonamido)pyridin-2-yl)butanamide;
2-(6-(cyclopropanesulfonamido)pyridin-2-yl)-N-(4-(6-ethoxypyrazin-2-yl)-2-fluorophenyl)butanamide;
2-(6-(cyclopropanesulfonamido)pyridin-2-yl)-N-(4-(6-ethoxypyrazin-2-yl)phenyl)butanamide;
2-(6-(cyclopropanesulfonamido)pyrazin-2-yl)-N-(4-(pyridin-3-yl)phenyl)acetamide;
2-(6-(ethylsulfonamido)pyrazin-2-yl)-N-(4-(pyridin-3-yl)phenyl)acetamide;
2-(6-(methylsulfonamido)pyrazin-2-yl)-N-(4-(pyridin-3-yl)phenyl)acetamide;
2-(6-(cyclopropanesulfonamido)pyrazin-2-yl)-N-(5-(6-ethoxypyrazin-2-yl)pyridin-2-yl)-2-methylpropanamide;
2-(6-(cyclopropanesulfonamido)pyrazin-2-yl)-N-(4-(6-ethoxypyrazin-2-yl)phenyl)-2-methylpropanamide;
4-(6-(cyclopropanesulfonamido)pyrazin-2-yl)-N-(5-(6-ethoxypyrazin-2-yl)pyridin-2-yl)tetrahydro-2H-pyran-4-carboxamide;
2-(6-(cyclopropanesulfonamido)pyrazin-2-yl)-N-(5-(6-ethoxypyrazin-2-yl)pyridin-2-yl)-4-methoxy-2-methylbutanamide;
N-(5-(6-ethoxypyrazin-2-yl)pyridin-2-yl)-4-methoxy-2-methyl-2-(6-(methylsulfonamido)pyrazin-2-yl)butanamide;
2-(6-(cyclopropanesulfonamido)pyrazin-2-yl)-N-(5-(6-ethoxypyrazin-2-yl)pyridin-2-yl)-2-fluorobutanamide;
2-(6-(cyclopropanesulfonamido)pyrazin-2-yl)-N-(5-(6-ethoxypyrazin-2-yl)pyridin-2-yl)butanamide;
2-(6-(cyclopropanesulfonamido)pyrazin-2-yl)-N-(4-(6-ethoxypyrazin-2-yl)phenyl)butanamide;
2-(6-(cyclopropanesulfonamido)pyrazin-2-yl)-N-(5-(6-ethoxypyrazin-2-yl)pyridin-2-yl)-2-methoxyacetamide;
2-(6-(cyclopropanesulfonamido)pyrazin-2-yl)-N-(4-(6-ethoxypyrazin-2-yl)phenyl)-2-methoxyacetamide;
2-(6-(cyclopropanesulfonamido)pyrazin-2-yl)-N-(5-(6-ethoxypyrazin-2-yl)pyridin-2-yl)-2-methoxypropanamide;
2-(6-(cyclopropanesulfonamido)pyrazin-2-yl)-N-(5-(6-ethoxypyrazin-2-yl)pyridin-2-yl)-2-(R)-fluorobutanamide;
2-(6-(cyclopropanesulfonamido)pyrazin-2-yl)-N-(5-(6-ethoxypyrazin-2-yl)pyridin-2-yl)-2-(S)-fluorobutanamide;
2-(4-(cyclopropanesulfonamido)pyrimidin-2-yl)-N-(5-(6-ethoxypyrazin-2-yl)pyridin-2-yl)butanamide;
N-(1-(2-(cyclopropanesulfonamido)pyrimidin-4-yl)cyclopropyl)-4-(6-ethoxypyrazin-2-yl)-2-fluorobenzamide;
N-(1-(2-(cyclopropanesulfonamido)pyrimidin-4-yl)propyl)-5-(6-ethoxypyrazin-2-yl)picolinamide;
N-(1-(2-(cyclopropanesulfonamido)pyrimidin-4-yl)propyl)-2-fluoro-4-(5-(trifluoromethyl)pyridin-3-yl)benzamide;
4-(5-chloropyridin-3-yl)-N-(1-(2-(cyclopropanesulfonamido)pyrimidin-4-yl)propyl)-2-fluorobenzamide;
N-(1-(2-(cyclopropanesulfonamido)pyrimidin-4-yl)propyl)-4-(5-(trifluoromethyl)pyridin-3-yl)benzamide;
4-(5-chloropyridin-3-yl)-N-(1-(2-(cyclopropanesulfonamido)pyrimidin-4-yl)propyl)benzamide;
N-(1-(2-(cyclopropanesulfonamido)pyrimidin-4-yl)propyl)-4-(6-ethoxypyrazin-2-yl)-2-(trifluoromethyl)benzamide;
N-(1-(2-(cyclopropanesulfonamido)pyrimidin-4-yl)propyl)-4-(6-ethoxypyrazin-2-yl)-2-fluorobenzamide;
N-(1-(2-(cyclopropanesulfonamido)pyrimidin-4-yl)propyl)-4-(6-(trifluoromethyl)pyrazin-2-yl)benzamide;
N-(1-(2-(cyclopropanesulfonamido)pyrimidin-4-yl)propyl)-4-(6-isopropoxypyrazin-2-yl)benzamide;
N-(1-(2-(cyclopropanesulfonamido)pyrimidin-4-yl)propyl)-4-(6-ethoxypyrazin-2-yl)benzamide;
N-(2-(2-(cyclopropanesulfonamido)pyrimidin-4-yl)butan-2-yl)-4-(6-ethoxypyrazin-2-yl)-2-fluorobenzamide;
N-(2-(6-(cyclopropanesulfonamido)pyrazin-2-yl) propan-2-yl)-2-fluoro-4-(6-isopropoxypyrazin-2-yl)benzamide;
N-(2-(6-(cyclopropanesulfonamido)pyrazin-2-yl)propan-2-yl)-4-(6-(trifluoromethyl)pyrazin-2-yl)benzamide;
N-(1-(6-(cyclopropanesulfonamido)pyrazin-2-yl)propyl)-4-(6-ethoxypyrazin-2-yl)-2-fluorobenzamide;
N-(1-(6-(cyclopropanesulfonamido)pyrazin-2-yl)propyl)-4-(6-ethoxypyrazin-2-yl)-2-(R)-fluorobenzamide; and
N-(1-(6-(cyclopropanesulfonamido)pyrazin-2-yl)propyl)-4-(6-ethoxypyrazin-2-yl)-2-(S)-fluorobenzamide;
   or a pharmaceutically acceptable salt and/or pharmaceutically acceptable solvate thereof.

Such CTPS1 inhibitors are disclosed in PCT publication number WO2019179652 which is incorporated by reference in its entirety for the purpose of the CTPS1 inhibitors disclosed therein. In particular a CTPS1 inhibitor may be a compound described in any one of clauses 1 to 148 of WO2019179652 or a pharmaceutically acceptable salt and/or pharmaceutically acceptable solvate thereof, in particular a compound P1 to P225 or a pharmaceutically acceptable salt and/or pharmaceutically acceptable solvate thereof.

Such CTPS1 inhibitors are also disclosed in PCT publication number WO2019180244 which is incorporated by reference in its entirety for the purpose of the CTPS1 inhibitors disclosed therein. In particular a CTPS1 inhibitor may be a compound described in any one of clauses 1 to 148 of WO2019180244 or a pharmaceutically acceptable salt and/or pharmaceutically acceptable solvate thereof, in particular a compound P1 to P225 or a pharmaceutically acceptable salt and/or pharmaceutically acceptable solvate thereof.

More suitably, the CTPS1 inhibitor is a compound of formula (IV):

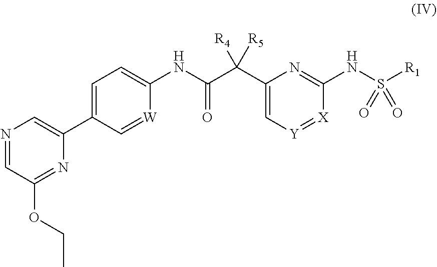

wherein:
(a) when R₄, R₅, X, Y and R₁ are as follows:

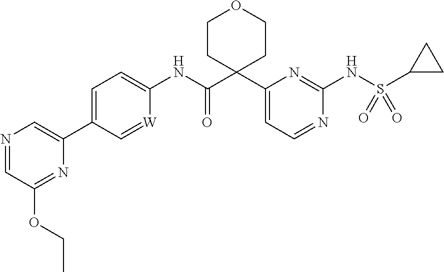

then W is N, CH or CF;
(b) when R₄, R₅, X, W and R₁ are as follows:

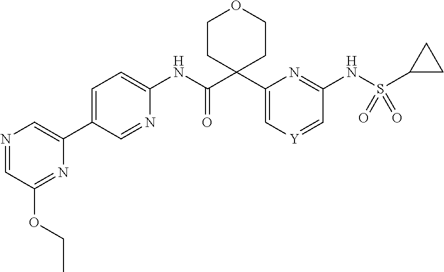

then Y is CH or N;
(c) when W, X, Y and R₁ are as follows:

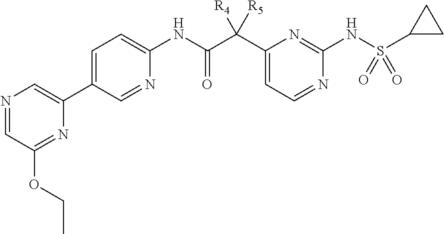

then R₄ and R₅ are joined to form the following structures:

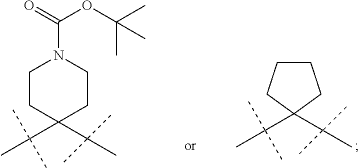

(d) when W, R₄, R₅, X and Y are as follows:

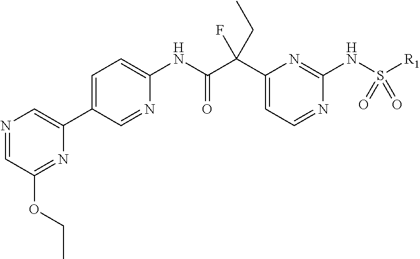

then R₁ is methyl or cyclopropyl; and
(e) the compound is selected from the group consisting of:

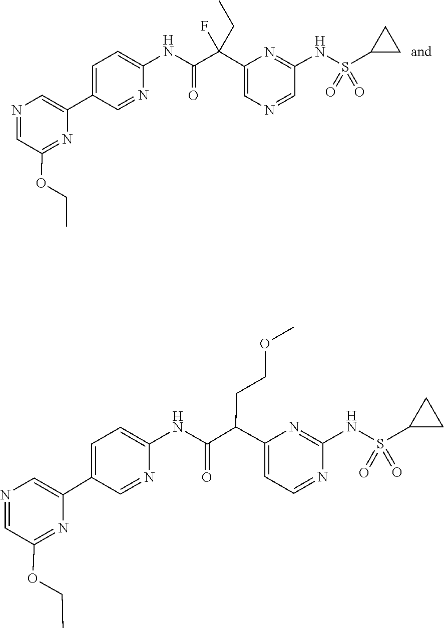 [SMILES: CCOc1cncc(-c2ccc(NC(=O)C(F)(CC)c3cncc(NS(=O)(=O)C4CC4)n3)nc2)n1 ; CCOc1cncc(-c2ccc(NC(=O)C(CCOC)c3ccnc(NS(=O)(=O)C4CC4)n3)nc2)n1]

or a pharmaceutically acceptable salt and/or pharmaceutically acceptable solvate thereof.

More suitably the CTPS1 inhibitor is selected from the following (‘List D’) compounds:
(R)-2-(2-(cyclopropanesulfonamido)pyrimidin-4-yl)-N-(5-(6-ethoxypyrazin-2-yl)pyridin-2-yl)-2-fluorobutanamide;
(S)-2-(2-(cyclopropanesulfonamido)pyrimidin-4-yl)-N-(5-(6-ethoxypyrazin-2-yl)pyridin-2-yl)-2-fluorobutanamide;
4-(2-(cyclopropanesulfonamido)pyrimidin-4-yl)-N-(5-(6-ethoxypyrazin-2-yl)pyridin-2-yl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclopentane-1-carboxamide;
4-(2-(cyclopropanesulfonamido)pyrimidin-4-yl)-N-(4-(6-ethoxypyrazin-2-yl)phenyl)tetrahydro-2H-pyran-4-carboxamide;
tert-butyl 4-(2-(cyclopropanesulfonamido)pyrimidin-4-yl)-4-((5-(6-ethoxypyrazin-2-yl)pyridin-2-yl)carbamoyl)piperidine-1-carboxylate;
4-(2-(cyclopropanesulfonamido)pyrimidin-4-yl)-N-(4-(6-ethoxypyrazin-2-yl)-2-fluorophenyl)tetrahydro-2H-pyran-4-carboxamide;
2-(2-(cyclopropanesulfonamido)pyrimidin-4-yl)-N-(5-(6-ethoxypyrazin-2-yl)pyridin-2-yl)-4-methoxybutanamide;
(R)—N-(5-(6-ethoxypyrazin-2-yl)pyridin-2-yl)-2-fluoro-2-(2-(methylsulfonamido)pyrimidin-4-yl)butanamide;
(S)—N-(5-(6-ethoxypyrazin-2-yl)pyridin-2-yl)-2-fluoro-2-(2-(methylsulfonamido)pyrimidin-4-yl)butanamide;
4-(6-(cyclopropanesulfonamido)pyridin-2-yl)-N-(5-(6-ethoxypyrazin-2-yl)pyridin-2-yl)tetrahydro-2H-pyran-4-carboxamide;
4-(6-(cyclopropanesulfonamido)pyrazin-2-yl)-N-(5-(6-ethoxypyrazin-2-yl) pyridin-2-yl)tetrahydro-2H-pyran-4-carboxamide;
(R)-2-(6-(cyclopropanesulfonamido)pyrazin-2-yl)-N-(5-(6-ethoxypyrazin-2-yl)pyridin-2-yl)-2-fluorobutanamide; and
(S)-2-(6-(cyclopropanesulfonamido)pyrazin-2-yl)-N-(5-(6-ethoxypyrazin-2-yl)pyridin-2-yl)-2-fluorobutanamide;
   or a pharmaceutically acceptable salt and/or a pharmaceutically acceptable solvate thereof.

Such CTPS1 inhibitors are disclosed in PCT publication number WO2020083975 which is incorporated by reference in its entirety for the purpose of the CTPS1 inhibitors disclosed therein. In particular a CTPS1 inhibitor may be a compound selected from P112, P113, P114, P115, P136, P137, P139, P143, P145, P165, P166, P186, P197, P206 and P207 or a pharmaceutically acceptable salt and/or pharmaceutically acceptable solvate thereof.

Alternatively, the CTPS1 inhibitor is a compound of formula (V):

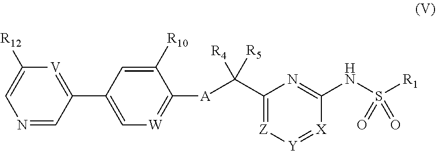

(a) when A, V, W, X, Y, Z, R₁, R₁₀ and R₁₂ are as follows:

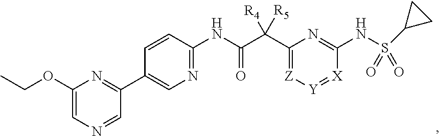

then R₄ and R₅ together with the carbon atom to which they attached form:

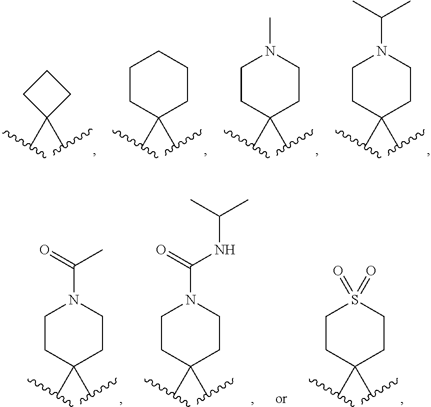

or
(b) when A, V, W, X, Y, Z, R₁, R₁₀ and R₁₂ are as follows:

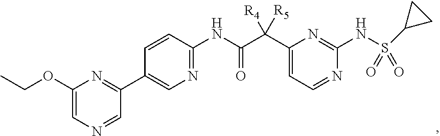

then R₄ and R₅ together with the carbon atom to which they are attached form:

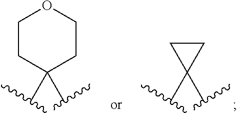 [SMILES: *C1(*)CCOCC1 ; *C1(*)CC1]

or
(c) when A, V, W, X, Y, Z, R₄, R₅, R₁₀ and R₁₂ are as follows:

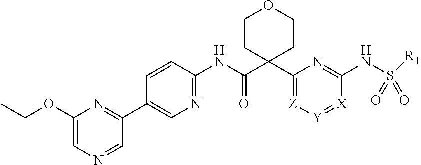

then R₁ is

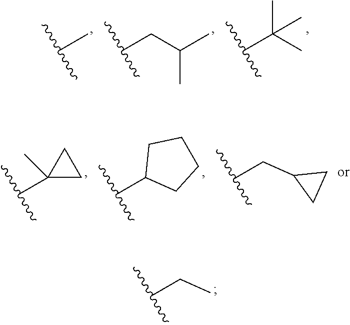

or
(d) when A, V, W, X, Y, Z, R₄, R₅, R₁₀ and R₁₂ are as follows:

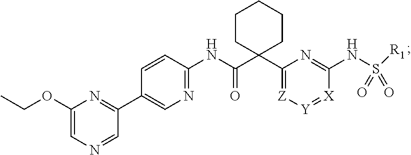

then R₁ is

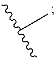

or
(e) when A, X, Y, Z, R₁, R₄ and R₅ are as follows:

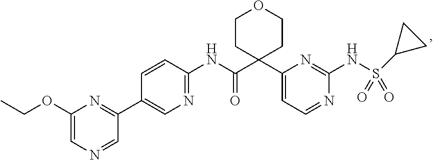 [SMILES: CCOc1cncc(-c2ccc(NC(=O)C3(c4ccnc(NS(=O)(=O)C5CC5)n4)CCOCC3)nc2)n1]

then V, W, R₁₀ and R₁₂ are:

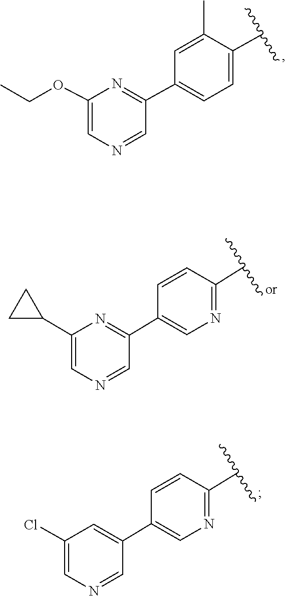 [SMILES: *c1ccc(-c2cncc(OCC)n2)cc1C ; *c1ccc(-c2cncc(C3CC3)n2)cn1 ; *c1ccc(-c2cncc(Cl)c2)cn1]

or
(f) when A, V, W, R₁, R₄, R₅, R₁₀ and R₁₂ are as follows:

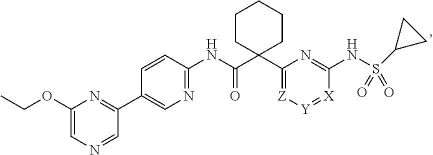

then Z, X and Y are

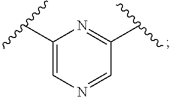 [SMILES: *c1cncc(*)n1]

or
(g) when A, V, W, R₁, R₄, R₅, R₁₀ and R₁₂ are as follows:

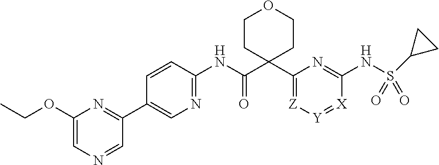

then Z, X and Y are

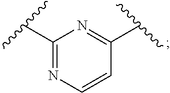 [SMILES: *c1ccnc(*)n1]

or
(h) when A, V, W, R₁, R₄, R₅, R₁₀ and R₁₂ are as follows

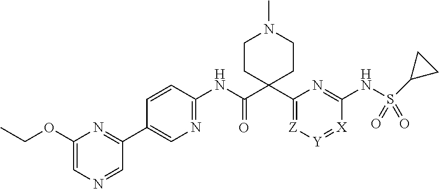

then Z, X and Y are

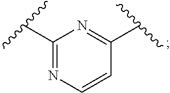 [SMILES: *c1ccnc(*)n1]

or a pharmaceutically acceptable salt and/or pharmaceutically acceptable solvate thereof.

More suitably the CTPS1 inhibitor is selected from the following (‘List E’) compounds:
N-(5-(6-ethoxypyrazin-2-yl)pyridin-2-yl)-4-(2-(methylsulfonamido)pyrimidin-4-yl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclohexane-1-carboxamide;
N-(5-(6-ethoxypyrazin-2-yl)pyridin-2-yl)-1-(2-(methylsulfonamido)pyrimidin-4-yl)cyclohexane-1-carboxamide;
1-(6-(cyclopropanesulfonamido)pyrazin-2-yl)-N-(5-(6-ethoxypyrazin-2-yl)pyridin-2-yl)cyclohexane-1-carboxamide;
4-(2-(cyclopropanesulfonamido)pyrimidin-4-yl)-N-(4-(6-ethoxypyrazin-2-yl)-2-methylphenyl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclobutane-1-carboxamide;
4-(4-(cyclopropanesulfonamido)pyrimidin-2-yl)-N-(5-(6-ethoxypyrazin-2-yl)pyridin-2-yl)tetrahydro-2H-pyran-4-carboxamide;
4-(2-(cyclopentanesulfonamido)pyrimidin-4-yl)-N-(5-(6-ethoxypyrazin-2-yl)pyridin-2-yl)tetrahydro-2H-pyran-4-carboxamide;
N-(5-(6-ethoxypyrazin-2-yl)pyridin-2-yl)-4-(2-((1-methylcyclopropane)-1-sulfonamido)pyrimidin-4-yl)tetrahydro-2H-pyran-4-carboxamide;
4-(2-(Cyclopropanesulfonamido)pyrimidin-4-yl)-N-(5-(6-ethoxypyrazin-2-yl)pyridin-2-yl)-1-methylpiperidine-4-carboxamide;
4-(2-(Cyclopropanesulfonamido)pyrimidin-4-yl)-N-(5-(6-ethoxypyrazin-2-yl)pyridin-2-yl)-1-isopropylpiperidine-4-carboxamide;
4-(2-(Cyclopropanesulfonamido)pyrimidin-4-yl)-N4-(5-(6-ethoxypyrazin-2-yl)pyridin-2-yl)-N1-isopropylpiperidine-1,4-dicarboxamide;
4-(2-((1,1-dimethylethyl)sulfonamido)pyrimidin-4-yl)-N-(5-(6-ethoxypyrazin-2-yl)pyridin-2-yl)tetrahydro-2H-pyran-4-carboxamide;
N-(4-(2-(cyclopropanesulfonamido)pyrimidin-4-yl)tetrahydro-2H-pyran-4-yl)-5-(6-ethoxypyrazin-2-yl)picolinamide;
1-Acetyl-4-(2-(cyclopropanesulfonamido)pyrimidin-4-yl)-N-(5-(6-ethoxypyrazin-2-yl)pyridin-2-yl)piperidine-4-carboxamide;
N-(5-(6-ethoxypyrazin-2-yl) pyridin-2-yl)-4-(2-((2-methylpropyl)sulfonamido)pyrimidin-4-yl)tetrahydro-2H-pyran-4-carboxamide;
4-(2-(cyclopropanesulfonamido)pyrimidin-4-yl)-N-(5-(6-cyclopropylpyrazin-2-yl) pyridin-2-yl)tetrahydro-2H-pyran-4-carboxamide;
N-(5′-chloro-[3,3′-bipyridin]-6-yl)-4-(2-(cyclopropanesulfonamido)pyrimidin-4-yl)tetrahydro-2H-pyran-4-carboxamide;
N-(1-(2-(cyclopropanesulfonamido)pyrimidin-4-yl)cyclopropyl)-5-(6-ethoxypyrazin-2-yl)picolinamide;
4-(2-(cyclopropanesulfonamido)pyrimidin-4-yl)-N-(5-(6-ethoxypyrazin-2-yl)pyridin-2-yl)tetrahydro-2H-thiopyran-4-carboxamide 1,1-dioxide;
N-(5-(6-ethoxypyrazin-2-yl) pyridin-2-yl)-4-(2-(ethylsulfonamido)pyrimidin-4-yl)tetrahydro-2H-pyran-4-carboxamide;
4-(2-(cyclopropylmethylsulfonamido)pyrimidin-4-yl)-N-(5-(6-ethoxypyrazin-2-yl)pyridin-2-yl)tetrahydro-2H-pyran-4-carboxamide and
4-(4-(cyclopropanesulfonamido)pyrimidin-2-yl)-N-(5-(6-ethoxypyrazin-2-yl)pyridin-2-yl)-1-methylpiperidine-4-carboxamide;
   or a pharmaceutically acceptable salt and/or pharmaceutically acceptable solvate thereof.

Such CTPS1 inhibitors are disclosed in PCT publication number WO2020245664 which is incorporated by reference in its entirety for the purpose of the CTPS1 inhibitors disclosed therein. In particular a CTPS1 inhibitor may be a compound selected from P319, P231 to P234, P236, P237, P238, P239, P240, P241, P243, P245, P246, P247, P249, P250, P252, P253, P257, P259, P262, P263 and P140 or a pharmaceutically acceptable salt and/or pharmaceutically acceptable solvate thereof.

Alternatively, the CTPS1 inhibitor is a compound of formula (VI):

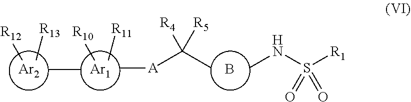

wherein ring B is selected from the group consisting of:

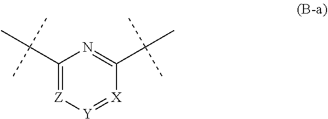

wherein X, Y and Z are as defined below; and

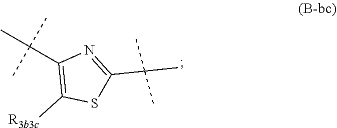

wherein R_{3b3c} is R_{3b} or R_{3c} as defined below;
   wherein when B is (B-a) the compound of formula (VI) is a compound of formula (VI-a):

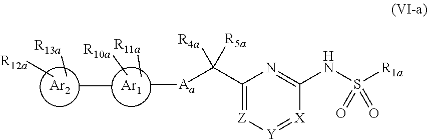

wherein:
Aₐ is Aₐₐ or A_{ba};
   wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂a;
Z is N or CR₃a,
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
   when Aₐ is —NHC(═O)— or —NHCH₂—;
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—;
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or
C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₅ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
   wherein
R₁ₐ is R₁ₐₐ; and/or
R₄ₐ and R₅ₐ are R₄ₐₐ and R₅ₐₐ; and/or
Aₐ is Aₐₐ; and
   wherein when B is (B-bc) and R_{3b3c} is R_{3b}, the compound of formula (VI) is a compound of formula (VI-b):

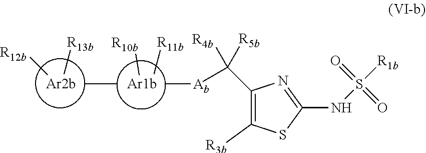

wherein:
A_{b} is A_{ab} or A_{bb};
   wherein:
      A_{ab} is —NR_{6b}CH₂— or —NR_{6b}—;
      A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆ heterocycloalkyl, C₁₋₃alkyleneOC₁₋₃alkyl, halo, OC₁₋₃haloalkyl, OC⁰⁻²alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆ cycloalkyl, C₀₋₂alkyleneC₃₋₆heterocycloalkyl, OC₁₋₆alkyl, OC₀₋₂alkyleneC₃₋₆ cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is 0 and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1 b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1 b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
   wherein:
R_{1b} is R_{1ab}; and/or
R_{4b} and R_{5b} are R_{4ab} and R_{5ab}; and/or
A is A_{ab}; or
   wherein when B is (B-bc) and R_{3b3c} is R_{3c}, the compound of formula (VI) is a compound of formula (VI-c):

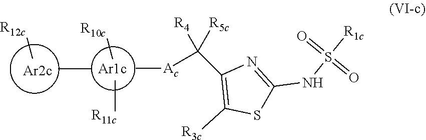

wherein:
A_{c} is A_{ac} or A_{bc};
   wherein:
   A_{ac} is —CH₂NR_{6c}—;
   A_{bc} is —C(═O)NR_{6c}—;
R_{1c} is R_{1ac} or R_{1bc};
   wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{23c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
wherein:
R_{1c} is R_{1ac}; and/or
R₄, and R₅, are R_{4ac} and R_{5ac}; and/or
A_{c} is A_{ac};
   or a pharmaceutically acceptable salt and/or pharmaceutically acceptable solvate thereof.

More suitably the CTPS1 inhibitor is selected from the following (‘List F’) compounds:
1-(2-(cyclopropanesulfonamido)pyrimidin-4-yl)-N-(5-(6-ethoxypyrazin-2-yl)pyridin-2-yl)-4-oxocyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 1);
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 2);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide;
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 2);
N-(4-(1-((4-(6-Ethoxypyrazin-2-yl)-2-fluorobenzyl)amino)propyl)pyrimidin-2-yl)cyclopropanesulfonamide;
1-(2-(cyclopropanesulfonamido)pyrimidin-4-yl)-N-(5-(6-ethoxypyrazin-2-yl)pyridin-2-yl)-4,4-difluorocyclohexane-1-carboxamide;
8-(2-(cyclopropanesulfonamido)pyrimidin-4-yl)-N-(5-(6-ethoxypyrazin-2-yl)pyridin-2-yl)-1,4-dioxaspiro[4.5]decane-8-carboxamide;
4-(2-((N,N-dimethylsulfamoyl)amino)pyrimidin-4-yl)-N-(5-(6-ethoxypyrazin-2-yl)pyridin-2-yl)tetrahydro-2H-pyran-4-carboxamide;
4-(2-(cyclopropanesulfonamido)pyrimidin-4-yl)-N-(5-(6-ethoxypyrazin-2-yl)pyridin-2-yl)-1-(methylsulfonyl)piperidine-4-carboxamide;
N-(4-(1-(((5-(6-ethoxypyrazin-2-yl)pyridin-2-yl)methyl)amino)cyclopropyl)pyrimidin-2-yl)cyclopropanesulfonamide;
N-(4-(1-((4-(6-ethoxypyrazin-2-yl)-2-fluorobenzyl)amino)cyclopropyl)pyrimidin-2-yl)cyclopropanesulfonamide;
N-(4-(4-(((4-(6-ethoxypyrazin-2-yl)phenyl)amino)methyl)tetrahydro-2H-pyran-4-yl)pyrimidin-2-yl)cyclopropanesulfonamide;
2-(2-(cyclopropanesulfonamido)pyrimidin-4-yl)-N-(5-(6-ethoxypyrazin-2-yl)pyridin-2-yl)-5,8-dioxaspiro[3.4]octane-2-carboxamide;
1-(2-(cyclopropanesulfonamido)pyrimidin-4-yl)-N-(5-(6-ethoxypyrazin-2-yl)pyridin-2-yl)-4-methoxycyclohexane-1-carboxamide;
N-(4-(1-((4-(6-ethoxypyrazin-2-yl)phenyl)amino)propyl)pyrimidin-2-yl)cyclopropanesulfonamidearboxamide;
4-(2-(Cyclopropanesulfonamido)pyrimidin-4-yl)-N-(5-(6-ethoxypyrazin-2-yl)pyridin-2-yl)-1-(2-methoxyacetyl)piperidine-4-carboxamide;
4-(2-(Cyclopropanesulfonamido)pyrimidin-4-yl)-N-(5-(6-ethoxypyrazin-2-yl)pyridin-2-yl)-1-(ethylsulfonyl)piperidine-4-carboxamide;
4-(2-(Cyclopropanesulfonamido)pyrimidin-4-yl)-1-(cyclopropylsulfonyl)-N-(5-(6-ethoxypyrazin-2-yl)pyridin-2-yl)piperidine-4-carboxamide;
4-(2-(Cyclopropanesulfonamido)pyrimidin-4-yl)-1-(N,N-dimethylsulfamoyl)-N-(5-(6-ethoxypyrazin-2-yl)pyridin-2-yl)piperidine-4-carboxamide;
4-(2-(Cyclopropanesulfonamido)pyrimidin-4-yl)-N-(5-(6-ethoxypyrazin-2-yl)pyridin-2-yl)-1-((trifluoromethyl)sulfonyl)piperidine-4-carboxamide;
4-(2-(Cyclopropanesulfonamido)pyrimidin-4-yl)-N-(5-(6-ethoxypyrazin-2-yl)pyridin-2-yl)-1-((1-methyl-1H-pyrazol-3-yl)sulfonyl)piperidine-4-carboxamide;
1-(cyanomethyl)-4-(2-(cyclopropanesulfonamido)pyrimidin-4-yl)-N-(5-(6-ethoxypyrazin-2-yl)pyridin-2-yl)piperidine-4-carboxamide;
ethyl 2-(4-(2-(cyclopropanesulfonamido)pyrimidin-4-yl)-4-((5-(6-ethoxypyrazin-2-yl)pyridin-2-yl)carbamoyl)piperidin-1-yl)acetate;
N-(5-(6-ethoxypyrazin-2-yl)pyridin-2-yl)-4-(2-(ethylsulfonamido)pyrimidin-4-yl)-1-(2-methoxyacetyl)piperidine-4-carboxamide;
N-(5-(6-ethoxypyrazin-2-yl)pyridin-2-yl)-4-(2-(ethylsulfonamido)pyrimidin-4-yl)-1-(methylsulfonyl)piperidine-4-carboxamide;
N-(5-(6-ethoxypyrazin-2-yl)pyridin-2-yl)-4-(2-(ethylsulfonamido)pyrimidin-4-yl)-1-(ethylsulfonyl)piperidine-4-carboxamide;
1-(Cyclopropylsulfonyl)-N-(5-(6-ethoxypyrazin-2-yl)pyridin-2-yl)-4-(2-(ethylsulfonamido)pyrimidin-4-yl)piperidine-4-carboxamide;
N-(5-(6-ethoxypyrazin-2-yl)pyridin-2-yl)-4-(2-(ethylsulfonamido)pyrimidin-4-yl)-1-((1-methyl-1H-pyrazol-3-yl)sulfonyl)piperidine-4-carboxamide;
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 1);
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 2);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 1);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 2);
4-amino-1-(2-(cyclopropanesulfonamido)pyrimidin-4-yl)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 2);
1-(2-(cyclopropanesulfonamido)pyrimidin-4-yl)-N-(5-(6-ethoxypyrazin-2-yl)pyridin-2-yl)-4-(methyl(oxetan-3-yl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 2);
1-(2-(cyclopropanesulfonamido)pyrimidin-4-yl)-4-((2,2-difluoroethyl)(methyl)amino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 2);
4-(6-(cyclopropanesulfonamido)pyrazin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(methylsulfonyl)piperidine-4-carboxamide;
4-(4-(cyclopropanesulfonamido)pyrimidin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(ethylsulfonyl)piperidine-4-carboxamide; and
4-(4-(cyclopropanesulfonamido)pyrimidin-2-yl)-N-(5-(6-ethoxypyrazin-2-yl)pyridin-2-yl)-1-(ethylsulfonyl)piperidine-4-carboxamide;
   or a pharmaceutically acceptable salt and/or pharmaceutically acceptable solvate thereof.

Such CTPS1 inhibitors are disclosed in PCT publication number WO2020245665 which is incorporated by reference in its entirety for the purpose of the CTPS1 inhibitors disclosed therein. In particular a CTPS1 inhibitor may be a compound described in any one of clauses 1 to 204 of WO2020245665 or a pharmaceutically acceptable salt and/or pharmaceutically acceptable solvate thereof, in particular a compound selected from P226, P227, P228, P229, P230, P235, P242, P244, P248, P251, P254, P255, P256, P258, P260, P261, P288, P289, P290, P291, P292, P293, P294, P295, P296, P297, P298, P299, P300, P301, P302, P303, P304, P305, P306, P307, P308, P309, P310, P311, P312, P313, P314, P315, P316, P317 and P318 or a pharmaceutically acceptable salt and/or pharmaceutically acceptable solvate thereof.

Alternatively, the CTPS1 inhibitor is a compound of formula (VII):

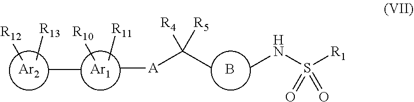

wherein
A is Aₐ or A_{b};
wherein
   Aₐ is an amine linker having the following structure: —NH—, —CH₂NH— or —NHCH₂—;
   A_{b} is an amide linker having the following structure: —C(═O)NH— or —NHC(═O)—;
B is

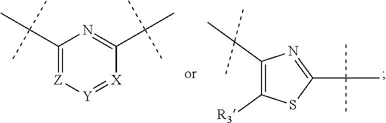

X is N or CH;
Y is N or CR₂;
Z is N or CR₃,
   with the proviso that when at least one of X or Z is N, Y cannot be N;
R₁ is C₁₋₅fluoroalkyl, with the proviso that R₁ is not CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R_{3′} is H, halo, CH₃, OC₁₋₂alkyl or CF₃; and
   when A is —NHC(═O)—, additionally R₃, together with R₅ forms a 5- or 6-membered cycloalkyl or 5 or 6 membered oxygen-containing heterocycloalkyl;
R₄ and R₅ are R₄ₐ and R₅ₐ, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—;
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—;
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃together with the nitrogen atom to which they are attached form a C₃₋₅heterocycloalkyl;
   or a pharmaceutically acceptable salt and/or pharmaceutically acceptable solvate thereof.

More suitably the CTPS1 inhibitor is selected from the following (‘List G’) compounds:
4-(2-((2,2-difluoroethyl)sulfonamido)pyrimidin-4-yl)-N-(5-(6-ethoxypyrazin-2-yl)pyridin-2-yl)tetrahydro-2H-pyran-4-carboxamide; and
2-(2-((2,2-difluoroethyl)sulfonamido)pyrimidin-4-yl)-N-(5-(6-ethoxypyrazin-2-yl)pyridin-2-yl)-2-fluorobutanamide;
   or a pharmaceutically acceptable salt and/or pharmaceutically acceptable solvate thereof.

Such CTPS1 inhibitors are disclosed in PCT publication number WO2021053403 which is incorporated by reference in its entirety for the purpose of the CTPS1 inhibitors disclosed therein. In particular a CTPS1 inhibitor may be a compound described in any one of clauses 1 to 191 of WO2021053403 or a pharmaceutically acceptable salt and/or pharmaceutically acceptable solvate thereof, in particular a compound selected from P271 and P284 or a pharmaceutically acceptable salt and/or pharmaceutically acceptable solvate thereof.

Alternatively, the CTPS1 inhibitor is compound of formula (VIII):

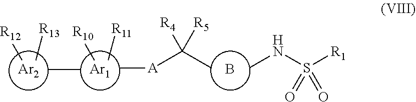

wherein
A is Aₐ or A_{b};
wherein
   Aₐ is an amine linker having the following structure: —NH—, —CH₂NH— or —NHCH₂—;
   A_{b} is an amide linker having the following structure: —C(═O)NH— or —NHC(═O)—;
B is

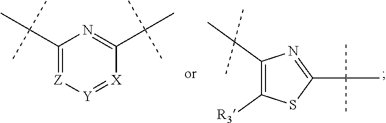

X is N or CH;
Y is N or CR₂;
Z is N or CR₃,
   with the proviso that when at least one of X or Z is N, Y cannot be N;
R₁ is C₁₋₅alkyl or C₀₋₂alkyleneC₃₋₅cycloalkyl, which alkyl or (alkylene)cycloalkyl is substituted by CN;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R_{3′} is H, halo, CH₃, OC₁₋₂alkyl or CF₃; and
   when A is —NHC(═O)—, additionally R₃ together with R₅ forms a 5- or 6-membered cycloalkyl or 5 or 6 membered oxygen-containing heterocycloalkyl;
R₄ and R₅ are R₄ₐ and R₅ₐ, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—;
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—;
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃together with the nitrogen atom to which they are attached form a C₃₋₅heterocycloalkyl;
   or a pharmaceutically acceptable salt and/or pharmaceutically acceptable solvate thereof.

More suitably the CTPS1 inhibitor is selected from the following (‘List H’) compounds:
4-(2-((1-cyanocyclopropane)-1-sulfonamido)pyrimidin-4-yl)-N-(5-(6-ethoxypyrazin-2-yl)pyridin-2-yl)tetrahydro-2H-pyran-4-carboxamide; and
4-(2-((cyanomethyl)sulfonamido)pyrimidin-4-yl)-N-(5-(6-ethoxypyrazin-2-yl)pyridin-2-yl)tetrahydro-2H-pyran-4-carboxamide;
   or a pharmaceutically acceptable salt and/or pharmaceutically acceptable solvate thereof.

Such CTPS1 inhibitors are disclosed in PCT publication number WO2021053402 which is incorporated by reference in its entirety for the purpose of the CTPS1 inhibitors disclosed therein. In particular a CTPS1 inhibitor may be a compound described in any one of clauses 1 to 191 of WO2021053402 or a pharmaceutically acceptable salt and/or pharmaceutically acceptable solvate thereof, in particular a compound selected from P285 and P287 or a pharmaceutically acceptable salt and/or pharmaceutically acceptable solvate thereof.

The CTPS1 inhibitor may be 4-(2-(cyclopropanesulfonamido)pyrimidin-4-yl)-N-(5-(6-ethoxypyrazin-2-yl)pyridin-2-yl)tetrahydro-2H-pyran-4-carboxamide (referred to herein as ‘CTPS-IA’):

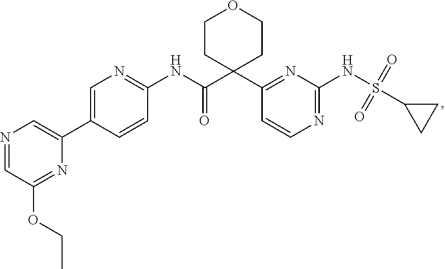 [SMILES: CCOc1cncc(-c2ccc(NC(=O)C3(c4ccnc(NS(=O)(=O)C5CC5)n4)CCOCC3)nc2)n1]

or a pharmaceutically acceptable salt and/or pharmaceutically acceptable solvate thereof.

Alternatively, the CTPS1 inhibitor may be N-(5-(6-ethoxypyrazin-2-yl)pyridin-2-yl)-4-(2-(ethylsulfonamido)pyrimidin-4-yl)tetrahydro-2H-pyran-4-carboxamide (referred to herein as ‘CTPS-IB’):

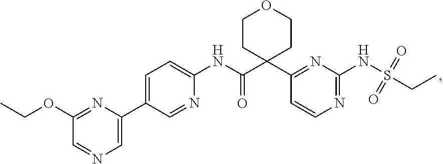 [SMILES: CCOc1cncc(-c2ccc(NC(=O)C3(c4ccnc(NS(=O)(=O)CC)n4)CCOCC3)nc2)n1]

or a pharmaceutically acceptable salt and/or pharmaceutically acceptable solvate thereof.

The compounds described above (and methods for making these compounds) are disclosed in PCT publication numbers WO2019106156, WO2019180244, WO2019106146, WO2019179652, WO2020245665, WO2020245664, WO2021053403, WO2021053402 or WO2020083975.

Depending on the nature of the specific CTPS1 inhibitor, the CTPS1 inhibitor may be provided in the form of a pharmaceutically acceptable salt and/or pharmaceutically acceptable solvate. In some embodiments the CTPS1 inhibitor is provided in the form of a pharmaceutically acceptable salt and pharmaceutically acceptable solvate (i.e. a pharmaceutically acceptable solvate of a pharmaceutically acceptable salt). In other embodiments the CTPS1 inhibitor is provided in the form of a pharmaceutically acceptable salt. In further embodiments the CTPS1 inhibitor is provided in the form of a pharmaceutically acceptable solvate. In some embodiments the CTPS1 inhibitor is provided in free form (i.e. not a salt or solvate).

Suitable pharmaceutically acceptable salts will be apparent to those skilled in the art. Pharmaceutically acceptable salts include those Remington's Pharmaceutical Sciences, 17th ed., Mack Publishing Company, Easton, PA, 1985, p. 1418. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Pharmaceutically acceptable salts may also be formed with metal ions such as metal salts, such as sodium or potassium salts, and organic bases such as basic amines e.g. with ammonia, meglumine, tromethamine, piperazine, arginine, choline, diethylamine, benzathine or lysine.

The CTPS1 inhibitor may form acid or base addition salts with one or more equivalents of the acid or base. The present invention includes within its scope all possible stoichiometric and non-stoichiometric forms.

The CTPS1 inhibitor may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

The CTPS1 inhibitor encompasses all isomers of the CTPS1 inhibitors disclosed herein including all geometric, tautomeric and optical forms, and mixtures thereof (e.g. racemic mixtures). Where additional chiral centres are present, the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The CTPS1 inhibitor encompasses all isotopic forms of the CTPS1 inhibitors provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

Unnatural variant isotopic forms comprising radioisotopes may, for example, be used for drug and/or substrate tissue distribution studies.

In one embodiment, the CTPS1 inhibitor is provided in a natural isotopic form.

In one embodiment, the CTPS1 inhibitor is provided in an unnatural variant isotopic form. In one embodiment, the CTPS1 inhibitor is provided whereby a single atom of the compound exists in an unnatural variant isotopic form. In another embodiment, the CTPS1 inhibitor is provided whereby two or more atoms exist in an unnatural variant isotopic form.

The CTPS1 inhibitor administered to a subject should be safe and effective, i.e. a CTPS1 inhibitor providing an acceptable balance of desired benefits and undesired side effects. “Safe and effective” is intended to include a compound that is effective to achieve a desirable effect in treatment of cancer. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms.

For avoidance of doubt, “safe and effective” as recited herein can be achieved by any suitable dosage regimen. Hence, for example, references herein to administering a safe and effective CTPS1 inhibitor, such as by a particular administration route, include achieving the safe and effective amount via a single dose or by plural doses, such as administered by the specified administration route. For instance, orally administering a safe and effective CTPS1 inhibitor includes both orally administering a single dose and orally administering any plural number of doses, provided that a safe and effective dose of CTPS1 inhibitor is thereby achieved by oral administration.

The CTPS1 inhibitor may be a compound of formula (IX):

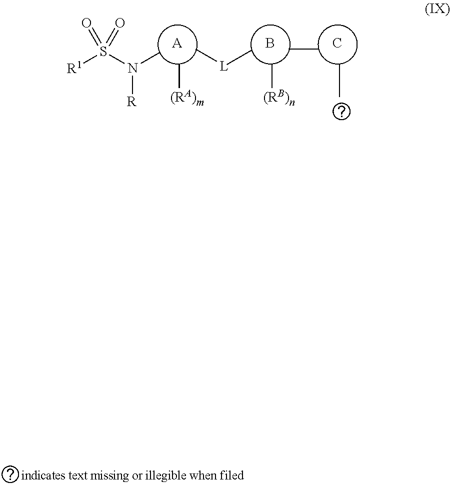

or a pharmaceutically acceptable salt thereof, wherein;
R¹ is selected from Cis aliphatic; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; each of which is substituted with q instances of R^{A};
Ring A is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur, a 3-7 membered saturated or partially unsaturated monocyclic heterocyclic ring having 1-2 heteroatoms independently selected from nitrogen, oxygen, and sulfur, and a 7-11 membered fused bicyclic heteroaryl ring having 1-4 heteroatoms independently selected from nitrogen, oxygen, and sulfur;
L is

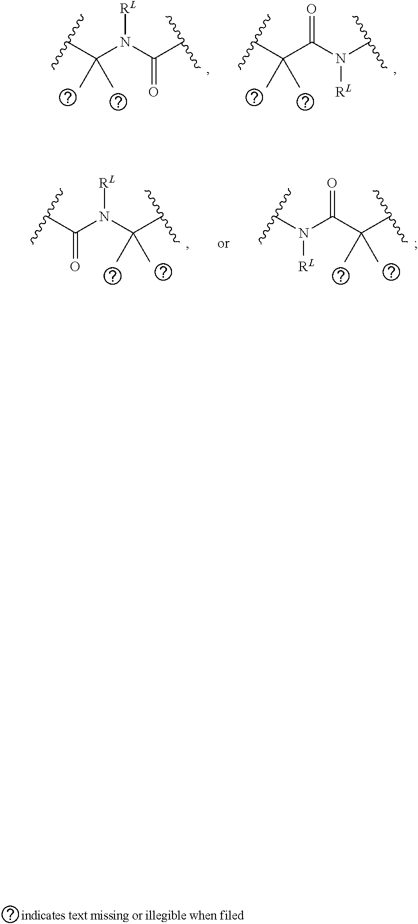

wherein each of R^{L}, R^{L′}, and R^{L″} is independently hydrogen, —CN, halogen, or an optionally substituted group selected from Cis aliphatic; phenyl; a 3-7 membered saturated or partially unsaturated monocyclic carbocyclic sing, a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or
two R^{L}, R^{L′}, and R^{L″} groups are taken together with the atoms to which each is attached, to form an optionally substituted 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; of
any one of R^{L}, R^{L′}, and R^{L″} together with Re forms a 7-10 membered saturated or partially unsaturated fused bicyclic ring;
Ring B is selected from phenyl, a 3-7 membered saturated or partially unsaturated monocyclic carbocyclic ring, a 5-6 membered monocyclic heteroaryl ring having 1-4 heteroatoms independently selected from nitrogen, oxygen, and sulfur, s 3-7 membered saturated or partially unsaturated monocyclic heterocyclic ring having 1-3 heteroatoms independently selected from nitrogen, oxygen, and sulfur; a 5-11 membered saturated or partially unsaturated fused, bridged, or spiro, bicycle carbocycle ring, a 7-11 membered fused bicycle aryl ring, a 7-11 membered saturated or partially unsaturated fused, bridged, or spiro, bicyclic heterocyclic ring having 1-4 heteroatoms independently selected from nitrogen, oxygen, and sulfur; and a 7-11 membered fused bicyclic heteroaryl ring having 1-4 heteroatoms independently selected from nitrogen, oxygen, and sulfur;
Ring C is selected from s phenyl, 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicycle heteroaryl ring having 1-4 heteroatoms independently selected from nitrogen, oxygen, and sulfur; or
the bond between Ring B and Ring C is absent, and Ring B and Ring C together form a 7-11 membered saturated or partially unsaturated fused, bridged, or spiro, bicyclic carbocyclic ring, a 7-11 membered fused bicyclic aryl ring; a 7-11 membered saturated or partially unsaturated fused, bridged, or spiro, bicyclic heterocyclic ring having 1-4 heteroatoms independently selected from nitrogen, oxygen, and sulfur; or a 7-11 membered fused bicycle heteroaryl ring having 1-4 heteroatoms independently selected from nitrogen, oxygen, and sulfur;
each instance of R^{A}, R^{B}, and R^{C} is independently oxo, halogen, —CN, —NO₂, —OR, —SR, —NR₂, —S(O)₂R,
—S(O)₂NR₂, —S(O)R, —S(O)NR₂, —C(O)R, —C(O)OR, —C(O)NR₂, —C(O)N(R)OR, —OC(O)R, —OC(O)NR₂, —N(R)(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R) OR or —P(O)R₂; or each instance of R^{C} is independently an optionally substituted group selected from C₁₋₆ aliphatic; phenyl, naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ting having 1-4 heteroatoms independently selected from nitrogen, oxygen, and sulfur, an 8-10 membered bicyclic heteroaryl ring having 1-5 heteroatoms independently selected from nitrogen, oxygen, and sulfur; & 5-8 membered saturated or partially unsaturated bridged bicyclic ring having 0-3 heteroatoms independently selected from nitrogen, oxygen, and sulfur; a 6-10 membered saturated or partially unsaturated spirocycle ring having 0.3 heteroatoms independently selected from nitrogen, oxygen, and sulfur; or a 6-11 membered saturated or partially unsaturated bicyclic heterocyclic ting having 1-2 heteroatoms independently selected from nitrogen, oxygen, and sulfur; each of which is substituted with r instances of R and s instances of R^{D}; or two R^{C} groups are optionally taken together with the atoms to which each RE is attached, to form an optionally substituted 3.7 membered saturated or partially unsaturated monocyclic carbocyclic ring; a 5-7 membered heteroaryl ring having 1-2 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur;
each instance of R^{D} is independently oxo, halogen, —CN, —NO₂, —OR, —SR, —NR₂, —S(O)₂R, —S(O)₂NR₂, —S(O)R, —S(O) NR₂, —C(O)R, —C(O)OR, —C(O)NR₂, —C(O)N(R)OR, —OC(O)R, —OC(O)NR₂, —N(R)C(O)OR, —N(R)C(O)OR, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring: a 3-7 membered saturated or partially unsaturated monocyclic
heterocycle ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur; an 8-10 membered bicycle heteroaryl ring having 1-4 heteroatoms independently selected from nitrogen, oxygen, and sulfur: a 7-12 membered saturated or partially unsaturated bicycle heterocyclic ting having 1-4 heteroatoms independently selected from nitrogen, oxygen, and sulfur; a 5-8 membered saturated of partially unsaturated bridged bicycle ring having 0-3 heteroatoms independently selected from nitrogen, oxygen, and sulfur, a 6-10 membered saturated or partially unsaturated spirocyclic ring having 0-3 heteroatoms independently selected from nitrogen, oxygen, and sulfur, a 6-11 membered saturated or partially unsaturated bicycle carbocyclic ring having 1-2 heteroatoms independently selected from nitrogen, oxygen, and sulfur; or:
two R groups are taken together with the atoms to which each R is attached, to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur, or
m is 0, 3, or 2,
n is 0, 1, or 2;
p is 0, 3, or 2;
each q is independently 0, 1, 2, 3, or 4;
each r is independently 0, 1, 2, 3, or 4; and
each s is independently 0, 1, 2, 3, or 4;
provided that when:
   R¹ is C₁₋₆ aliphatic or a 3-7 membered saturated or partially unsaturated monocyclic carbocyclic ring;
   the R group of the sulfonamide moiety

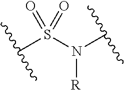

is hydrogen or para-methoxybenzyl;
L is

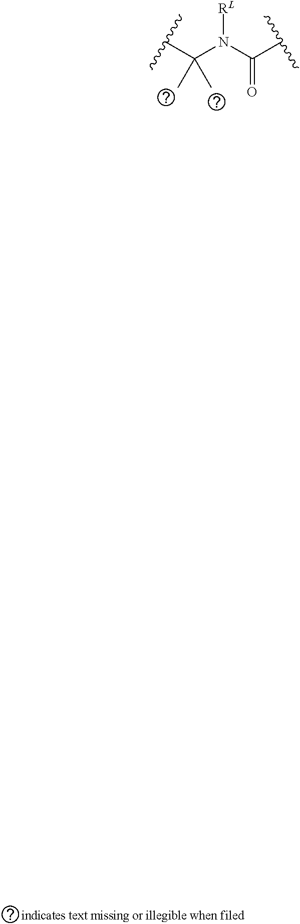

and the R^{L} and R^{L′} or R_{L} and R^{L′} groups are not taken together with the atoms to which each is attached to form an optionally substituted 3-7 membered saturated or partially unsaturated monocyclic heterocyclic ring having 1-2 heteroatoms independently selected from nitrogen, oxygen, and sulfur, or L is

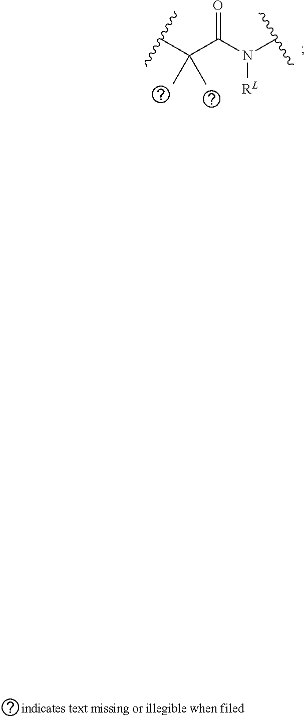

Ring B is phenyl or a 6-membered monocyclic heteroaryl ring having 1-4 heteroatoms independently selected from nitrogen, oxygen, and sulfur; and
Ring C is phenyl or a 6-membered monocyclic heteroaryl ring having 1-4 heteroatoms independently selected from nitrogen, oxygen, and sulfur and is attached to Ring B in the para position relative to the L group;
   then Ring A and its R^{A} substituents are other than

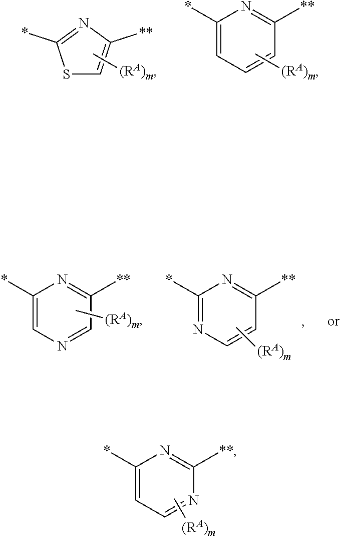

where * denotes attachment to the

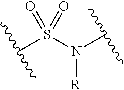

moiety and ** denotes attachment to the

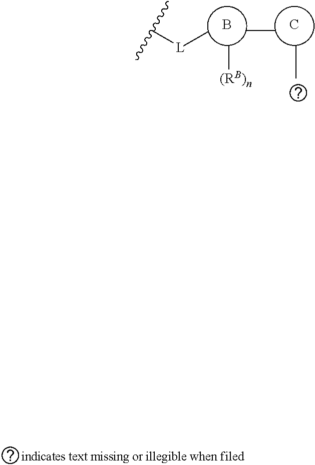

moiety.
   or a pharmaceutically acceptable salt and/or pharmaceutically acceptable solvate thereof.

Such CTPS1 inhibitors are disclosed in PCT publication number WO2022087634 which is incorporated by reference in its entirety for the purpose of the CTPS1 inhibitors disclosed therein. In particular a CTPS1 inhibitor may be a compound described in any one of claims 1 to 31 of WO2022087634 or a pharmaceutically acceptable salt and/or pharmaceutically acceptable solvate thereof. A CTPS1 inhibitor may be a compound selected from compounds I-1 to 1-286 of WO2022087634, or a pharmaceutically acceptable salt and/or pharmaceutically acceptable solvate thereof. A CTPS1 inhibitor may be a compound selected from compounds Z-1 to Z-10 of WO2022087634, or a pharmaceutically acceptable salt and/or pharmaceutically acceptable solvate thereof.

In one embodiment the CTPS1 inhibitor is not a CTPS1 inhibitor disclosed in PCT publication number WO2022087634. In a further embodiment, the CTPS1 inhibitor is not (i) a compound described in any one of claims 1 to 31 of WO2022087634 or a pharmaceutically acceptable salt and/or pharmaceutically acceptable solvate thereof, (ii) a compound selected from compounds I-1 to 1-286 of WO2022087634, or a pharmaceutically acceptable salt and/or pharmaceutically acceptable solvate thereof, or (iii) a compound selected from compounds Z-1 to Z-10 of WO2022087634, or a pharmaceutically acceptable salt and/or pharmaceutically acceptable solvate thereof.

CTPS1 inhibitors are disclosed in WO2022/087634, which is incorporated by reference in its entirety for the purpose of defining CTPS1 inhibitors. In some embodiments the CTPS1 inhibitor is as described in WO2022/087634, such as any of compounds I-1 to I-286 or Z-1 to Z-10, or a pharmaceutically acceptable salt and/or pharmaceutically acceptable solvate thereof. In other embodiments the CTPS1 inhibitor is not described in WO2022/087634.

Suitably, the CTPS1 inhibitor is not:
1. A compound of formula I:

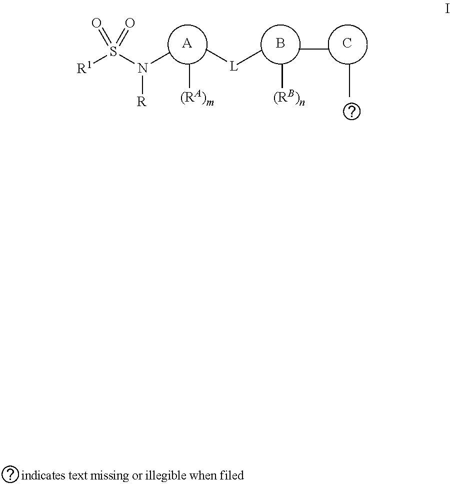

or a pharmaceutically acceptable salt thereof, wherein;
R¹ is selected from C₁₋₆ aliphatic; a 3-7 membered saturated or partially unsaturated monocyclic carbocyclic ring; and a 3-7 membered saturated or partially unsaturated monocyclic heterocyclic ring having 1-2 heteroatoms independently selected front nitrogen, oxygen, and sulfur; each of which is substituted with q instances of R^{A};
Ring A is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur, a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur;
L is

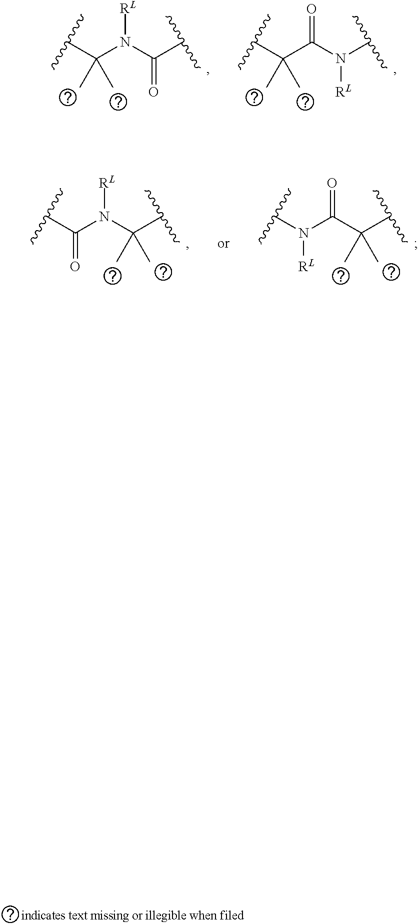

wherein each of R^{L}, R^{L′}, and R^{L″} is independently hydrogen, —CN, halogen, or an optionally substituted group selected from C₁₋₆ aliphatic; phenyl, a 3-7 membered saturated or partially unsaturated monocyclic carbocyclic sing, a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or
two of R^{L}, R^{L′} and R^{L″} groups are taken together with the atoms to which each is attached, to form an optionally substituted 3-7 membered saturated or partially unsaturated monocyclic carbocyclic ring; or a 3-7 membered saturated or partially unsaturated monocyclic heterocyclic ding having 1-2 heteroatoms independently selected from nitrogen, oxygen, and sulfur; or
any one of R^{L}, R^{L′}, and R^{L″}, together with R^{B} forms a 7-10 membered saturated or partially unsaturated fused bicyclic ring;
Ring B is selected from phenyl, a 3-7 membered saturated or partially unsaturated monocyclic carbocyclic ring; a 5-6 membered monocycle heteraryl ring having 1-4 heteroatoms independently selected from nitrogen, oxygen, and sulfur; a 3-7 membered saturated or partially unsaturated monocyclic heterocyclic ring having 1-3 heteroatoms independently selected from nitrogen, oxygen, and sulfur; a 5-13 membered saturated or partially unsaturated fused, bridged, or spiro, bicyclic carbocycle ring, a 7-11 membered fused bicyclic aryl ring, a 7-11 membered saturated of partially unsaturated fused, bridged, or spiro, bicyclic heterocyclic ring having 1-4 heteroatoms independently selected from nitrogen, oxygen, and sulfur; and a 7-11 membered fused bicycle heteroaryl ting having 1-4 heteroatoms independently selected from nitrogen, oxygen, and sulfur;
Ring C is selected from a phenyl, 3-7 membered saturated or partially unsaturated monocyclic carbocyclic ring, a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur, or
the bond between Ring B and Ring C is absent, and Ring B and Ring C together form a 7-11 membered saturated or partially unsaturated fused, bridged, or spiro, bicyclic carbocyclic ring; a 7-11 membered fused bicyclic aryl ring; a 7-11 membered saturated or partially unsaturated fused, bridged, or spiro, bicyclic heterocyclic ring having 1-4 heteroatoms independently selected from nitrogen, oxygen, and sulfur; or a 7-11 membered fused bicyclic heteroaryl ring having 1-4 heteroatoms independently selected from nitrogen, oxygen, and sulfur;
each instance of R^{A}, R^{B}, and R^{C} is independently oxo, halogen, —CN, —NO₂, —OR, —SR, —NR₂, —S(O)₂R, —SO₂NR₂, —S(O)R, —S(O)NR₂, C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{C} is independently an optionally substituted group selected from C₁₋₆ aliphatic, phenyl, naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur, an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ting having 1-3 heteroatoms independently selected from nitrogen, oxygen, and sulfur; each of which is substituted with r instances of R and s instances of R^{D}; or two R^{C} groups are optionally taken together with the atoms to which each R^{C} is attached to form an optionally substituted 3-7 membered saturated or partially unsaturated monocyclic carbocyclic ring, a 5-7 membered heteroaryl ring having 1-2 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur;
each instance of R^{D} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂;
each R is independently hydrogen, —CN, halogen, or an optionally substituted group selected from C₁₋₆ aliphatic; phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, an 8-10 membered bicyclic heteraryl ring having 1-4 heteroatoms independently selected from nitrogen, oxygen, and sulfur; a 7-12 membered Saturated or partially unsaturated bicycle heterocyclic ding having 1-4 heteroatoms independently selected from nitrogen, oxygen, and sulfur; a 5-8 membered saturated of partially unsaturated bridged bicyclic ring having 0-3 heteroatoms independently selected from nitrogen, oxygen, and sulfur, a 6-10 membered saturated or partially unsaturated spirocyclic ring having 0-3 heteroatoms independently selected from nitrogen, oxygen, and sulfur; a 6-11 membered saturated ex partially unsaturated bicyclic carbocyclic ring having 3-2 heteroatoms independently selected from nitrogen, oxygen, and sulfur; or:
two R groups are taken together with the atoms to which each R is attached, to form an optionally substituted 3-7 membered saturated or partially unsaturated monocyclic carbocyclic ring, a 3-7 membered saturated or partially unsaturated monocyclic heterocyclic ring having 1-2 heteroatoms independently selected from nitrogen, oxygen, and sulfur; of
m is 0, 1, or 2;
n is 0, 3, or 2;
p is 0, 1, or 2;
each q is independently 0, 1, 2, 3, or 4;
each r is independently 0, 1, 2, 3, or 4; and
each s is independently 0, 1, 2, 3, or 4;
provided that when:
   R¹ is C₁₋₆ aliphatic or a 3-7 membered saturated or partially unsaturated monocyclic carbocyclic ring;
   the R group of the sulfonamide moiety

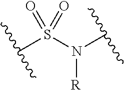

is hydrogen of pars-methoxybenzyl,
L is

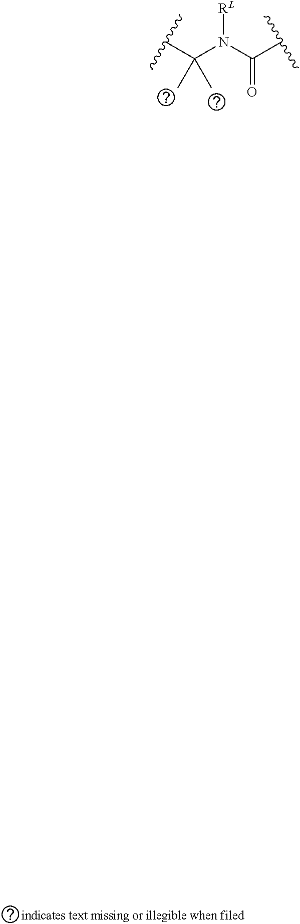

and the R^{L} and R^{L′} or R^{L} and R^{L″} groups are not taken together with the atoms to which each is attached to form an optionally substituted 3-7 membered saturated or partially unsaturated monocylic heterocyclic ring having 1-2 heteroatoms independently selected from nitrogen, oxygen, and sulfur, or L is

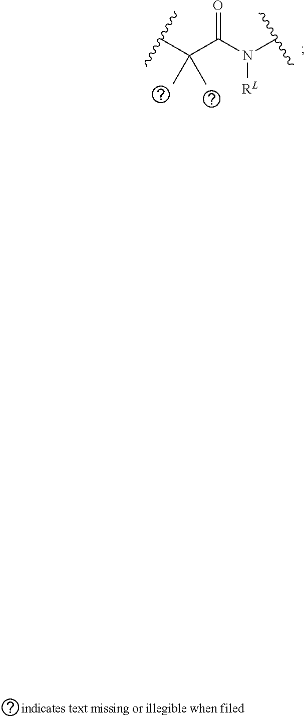

Ring B is phenyl or a 6-membered monocyclic heteroaryl ring having 1-4 heteroatoms independently selected fro nitrogen, oxygen, and sulfur, and
Ring C is phenyl or a 6-membered monocycle heteroaryl ring having 1-4 heteroatoms independently selected from nitrogen, oxygen, and sulfur and is attached to Ring B in the para position relative to the L group;
   then Ring A and its R^{A} substituents are other than

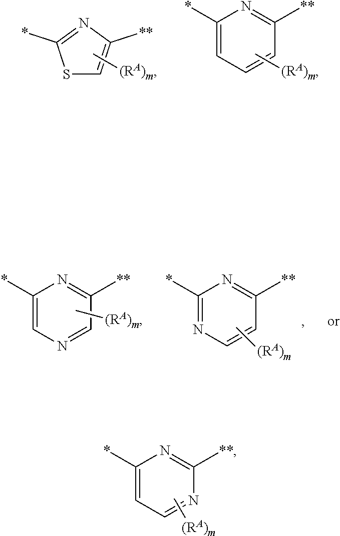

where * denotes attachment to the

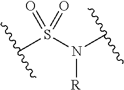

moiety and ** denotes attachment to the

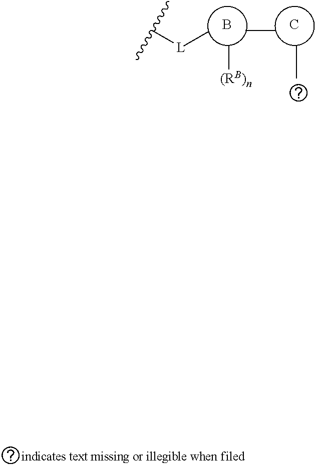

moiety.

Suitably, the CTPS1 inhibitor is not a CTPS1 inhibitor as defined in claim 1 of WO2022/087634. Suitably, the CTPS1 inhibitor is not a CTPS1 inhibitor as defined in WO2022/087634.

### BCL2 Inhibitors

In one aspect of the invention there is provided a BCL2 inhibitor for use in the treatment of cancer with a CTPS1 inhibitor.

A BCL2 inhibitor, as used herein, is an agent which directly inhibits BCL2 activity, such as BCL2 induced inhibition of apoptosis. Direct inhibition of BCL2 may be quantified using any suitable the assay procedure, though is suitably performed using the procedure set out in Example 3.

The activity of the BCL2 inhibitors has been determined using competitive fluorescence polarisation and time-resolved fluorescence resonance energy transfer binding affinity assays to calculate inhibitory constant (Ki) values for binding to BCL2 to determine potency, and related proteins such as BCL2L1 to determine specificity (Souers 2013; Tse 2008; Zhang 2002).

BCL2 inhibitors of particular interest are those demonstrating Ki values for binding to BCL2 of 50 nM or lower, such as 20 nM or lower, such as 10 nM or lower, such as 5 nM or lower, such as 1 nM or lower.

BCL2 inhibitors of particular interest are those demonstrating Ki values for binding to BCL2 of 50 nM or lower, such as 20 nM or lower, such as 10 nM or lower, such as 5 nM or lower, such as 1 nM or lower using the assay procedure set out in Example 3.

BCL2 inhibitors of particular interest are those demonstrating a selectivity for BCL2 over BCL2L1 of >2-fold, such as >5-fold, such as >10-fold, for example a selectivity for BCL2 over BCL2L1 of >2-fold, such as >5-fold, such as >10-fold using the assay procedure set out in Example 4.

Further BCL2 inhibitors of particular interest are those demonstrating a selectivity for BCL2 over MCL1 of >2-fold, such as >5-fold, such as >10-fold, for example a selectivity for BCL2 over BCL2L1 of >2-fold, such as >5-fold, such as >10-fold using the assay procedure set out in Example 4.

In the case of medicaments intended for human use, BCL2 inhibition and BCL2 vs BCL2L1 selectivity should be based on human forms of the proteins.

Suitably the BCL2 inhibitor is a BH-3 mimetic. A BH-3 mimetic is an agent which mimics the activity of the BH3 domain of pro-apoptotic proteins, binding to and blocking the anti-apoptotic BCL2 protein. Particular BH-3 mimetics include the following (clinical trial data is sourced from .clinicaltrials.gov accessed 21 Dec. 2021):

### Venetoclax

Venetoclax (4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide, CAS number 1257044-40-8, trade name Venclyxto®) is a BCL2 specific BH3-mimetic. It is FDA approved as a single agent or in combination with an anti-CD20 monoclonal antibody for the treatment of chronic lymphocytic leukaemia and small lymphocytic lymphoma, and in combination with azacitidine, decitabine, or low-dose cytarabine for the treatment of acute myeloid leukaemia. Venetoclax is typically administered orally once daily up to a dose of 400 to 600 mg. Of 197 clinical trials of venetoclax currently recruiting, 97.5% are exclusively enrolling haematological malignancies and 2.5% are enrolling patients with solid tumours. The structure of venetoclax is provided below.

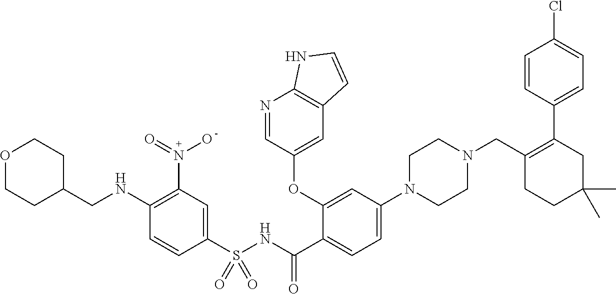

The BCL2 inhibitor may therefore be ventoclax or a pharmaceutically acceptable salt and/or a pharmaceutically acceptable solvate thereof. In one embodiment the BCL2 inhibitor is venetoclax. In one embodiment the BCL2 inhibitor is a pharmaceutically acceptable salt of venetoclax. In one embodiment the BCL2 inhibitor is a pharmaceutically acceptable solvate of venetoclax. In one embodiment the BCL2 inhibitor is a pharmaceutically acceptable salt and a pharmaceutically acceptable solvate of venetoclax.

In one embodiment, the BCL2 inhibitor is not venetoclax. Suitably, the BCL2 inhibitor is not venetoclax or a pharmaceutically acceptable salt and/or a pharmaceutically acceptable solvate thereof.

Venetoclax is disclosed in Park 2008, which is incorporated herein by reference in its entirety for the purpose of defining the BCL2 inhibitor.

### Navitoclax

Navitoclax (4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide, CAS number 923564-51-6, also known as ABT-263) is a BH3-mimetic with activity against BCL2L1 (BCL-XL) as well as BCL2. It has yet to be approved in any indication. Clinical trials are currently recruiting patients with haematological and solid tumours. It is typically administered orally. Clinical development has been hampered by thrombocytopenia due to inhibition of BCL2L1. The structure of navitoclax is provided below.

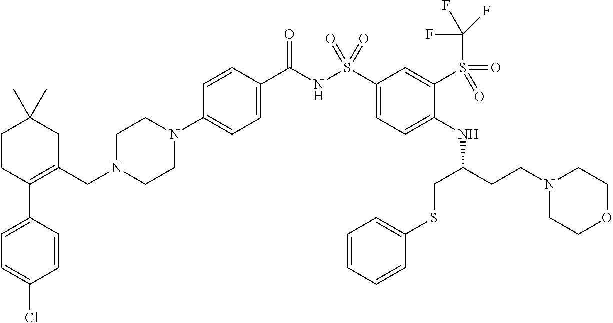 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

The BCL2 inhibitor may therefore be navitoclax or a pharmaceutically acceptable salt and/or a pharmaceutically acceptable solvate thereof. In one embodiment the BCL2 inhibitor is navitoclax. In one embodiment the BCL2 inhibitor is a pharmaceutically acceptable salt of navitoclax. In one embodiment the BCL2 inhibitor is a pharmaceutically acceptable solvate of navitoclax. In one embodiment the BCL2 inhibitor is a pharmaceutically acceptable salt and a pharmaceutically acceptable solvate of navitoclax.

Navitoclax is disclosed in Tse 2008, which is incorporated herein by reference in its entirety for the purpose of defining the BCL2 inhibitor.

### Obatoclax

Obatoclax ((2Z)-2-[(5Z)-5-[(3,5-dimethyl-1H-pyrrol-2-yl)methylidene]-4-methoxypyrrol-2-ylidene]indole, CAS number 803712-67-6, also known as GX15-070) is a BCL2 specific BH3-mimetic that has previously been tested in clinical trials in a range of haematological and solid tumour indications. It has yet to be approved in any condition and there are no clinical trials of this agent currently recruiting. The structure of obatoclax is provided below.

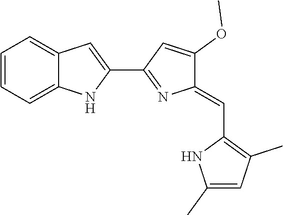 [SMILES: COC1=CC(c2cc3ccccc3[nH]2)=N/C1=C\c1[nH]c(C)cc1C]

The BCL2 inhibitor may therefore be obatoclax or a pharmaceutically acceptable salt and/or a pharmaceutically acceptable solvate thereof. In one embodiment the BCL2 inhibitor is obatoclax. In one embodiment the BCL2 inhibitor is a pharmaceutically acceptable salt of obatoclax. In one embodiment the BCL2 inhibitor is a pharmaceutically acceptable solvate of obatoclax. In one embodiment the BCL2 inhibitor is a pharmaceutically acceptable salt and a pharmaceutically acceptable solvate of obatoclax.

Obatoclax is disclosed in Li 2008, which is incorporated herein by reference in its entirety for the purpose of defining the BCL2 inhibitor.

### BCL201

BCL201 (also known as S55746) is a BCL2 specific BH3-mimetic that has previously been tested in a small number of clinical trials of haematological tumours. It has yet to be approved in any condition and there are no clinical trials of this agent currently recruiting. The structure of BCL201 is provided below.

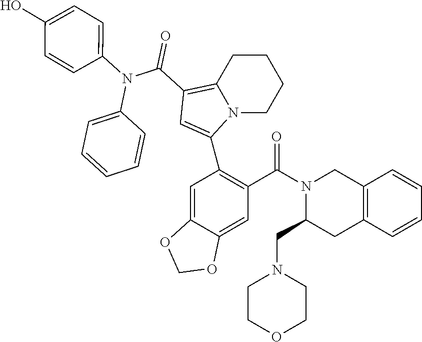 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

The BCL2 inhibitor may therefore be BCL201 or a pharmaceutically acceptable salt and/or a pharmaceutically acceptable solvate thereof. In one embodiment the BCL2 inhibitor is BCL201. In one embodiment the BCL2 inhibitor is a pharmaceutically acceptable salt of BCL201. In one embodiment the BCL2 inhibitor is a pharmaceutically acceptable solvate of BCL201. In one embodiment the BCL2 inhibitor is a pharmaceutically acceptable salt and a pharmaceutically acceptable solvate of BCL201.

BCL201 is disclosed in Casara 2018 which is incorporated herein by reference in its entirety for the purpose of defining the BCL2 inhibitor.

### AT101

AT101 (also known as gossypol) is a BH3-mimetic with activity against BCL2L1 (BCL-XL) and MCL1 as well as BCL2 that has previously been tested in clinical trials in a range of haematological and solid tumour indications. It has yet to be approved in any condition and there are no clinical trials of this agent currently recruiting. The structure of AT101 is provided below.

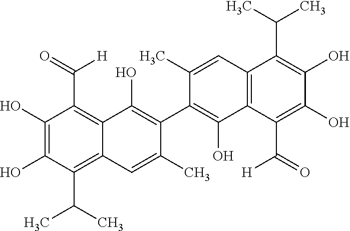 [SMILES: Cc1cc2c(C(C)C)c(O)c(O)c(C=O)c2c(O)c1-c1c(C)cc2c(C(C)C)c(O)c(O)c(C=O)c2c1O]

The BCL2 inhibitor may therefore be AT101 or a pharmaceutically acceptable salt and/or a pharmaceutically acceptable solvate thereof. In one embodiment the BCL2 inhibitor is AT101. In one embodiment the BCL2 inhibitor is a pharmaceutically acceptable salt of AT101. In one embodiment the BCL2 inhibitor is a pharmaceutically acceptable solvate of AT101. In one embodiment the BCL2 inhibitor is a pharmaceutically acceptable salt and a pharmaceutically acceptable solvate of AT101.

AT101 is disclosed in Wang 2006 which is incorporated herein by reference in its entirety for the purpose of defining the BCL2 inhibitor.

In one embodiment, the BCL2 inhibitor is selected from the group consisting of venetoclax, navitoclax, obatoclax, BCL201 and AT101, pharmaceutically acceptable salts and/or pharmaceutically acceptable solvates of any thereof. More suitably the BCL2 inhibitor is selected from the group consisting of venetoclax, navitoclax, obatoclax, BCL201 and AT101, pharmaceutically acceptable salts and/or pharmaceutically acceptable solvates of any thereof. More suitably the BCL2 inhibitor is selected from the list consisting of venetoclax, obatoclax, BCL201 and AT101, pharmaceutically acceptable salts and/or pharmaceutically acceptable solvates of any thereof. More suitably the BCL2 inhibitor is venetoclax, or a pharmaceutically acceptable salt and/or pharmaceutically acceptable solvate thereof.

Depending on the nature of the specific BCL2 inhibitor, the BCL2 inhibitor may be provided in the form of a pharmaceutically acceptable salt and/or pharmaceutically acceptable solvate. In some embodiments the BCL2 inhibitor is provided in the form of a pharmaceutically acceptable salt and pharmaceutically acceptable solvate (i.e. a pharmaceutically acceptable solvate of a pharmaceutically acceptable salt). In other embodiments the BCL2 inhibitor is provided in the form of a pharmaceutically acceptable salt. In further embodiments the BCL2 inhibitor is provided in the form of a pharmaceutically acceptable solvate. In some embodiments the BCL2 inhibitor is provide in free form (i.e. not a salt or solvate).

Suitable pharmaceutically acceptable salts will be apparent to those skilled in the art. Pharmaceutically acceptable salts include those Remington's Pharmaceutical Sciences, 17th ed., Mack Publishing Company, Easton, PA, 1985, p. 1418. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Pharmaceutically acceptable salts may also be formed with metal ions such as metal salts, such as sodium or potassium salts, and organic bases such as basic amines e.g. with ammonia, meglumine, tromethamine, piperazine, arginine, choline, diethylamine, benzathine or lysine.

The BCL2 inhibitor may form acid or base addition salts with one or more equivalents of the acid or base. The present invention includes within its scope all possible stoichiometric and non-stoichiometric forms.

The BCL2 inhibitor may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

The BCL2 inhibitor encompasses all isomers of the BCL2 inhibitors disclosed herein including all geometric, tautomeric and optical forms, and mixtures thereof (e.g. racemic mixtures). Where additional chiral centres are present, the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The BCL2 inhibitor encompasses all isotopic forms of the BCL2 inhibitors provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

Unnatural variant isotopic forms comprising radioisotopes may, for example, be used for drug and/or substrate tissue distribution studies.

In one embodiment, the BCL2 inhibitor is provided in a natural isotopic form.

In one embodiment, the BCL2 inhibitor is provided in an unnatural variant isotopic form.

In one embodiment, the BCL2 inhibitor is provided whereby a single atom of the compound exists in an unnatural variant isotopic form. In another embodiment, the BCL2 inhibitor is provided whereby two or more atoms exist in an unnatural variant isotopic form.

In general, the BCL2 inhibitors disclosed herein may be made according to the organic synthesis techniques known to those skilled in this field. For example, preparation of venetoclax is described in Park 2008, which is incorporated herein by reference in its entirety for the purpose of methods of producing the BCL2 inhibitor venetoclax as disclosed herein.

The BCL2 inhibitor administered to a subject should be safe and effective, i.e. a BCL2 inhibitor providing an acceptable balance of desired benefits and undesired side effects. “Safe and effective” is intended to include a compound that is effective to achieve a desirable effect in treatment of cancer. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms.

For avoidance of doubt, “safe and effective” as recited herein can be achieved by any suitable dosage regimen. Hence, for example, references herein to administering a safe and effective BCL2 inhibitor, such as by a particular administration route, include achieving the safe and effective amount via a single dose or by plural doses, such as administered by the specified administration route. For instance, orally administering a safe and effective BCL2 inhibitor includes both orally administering a single dose and orally administering any plural number of doses, provided that a safe and effective dose of BCL2 inhibitor is thereby achieved by oral administration.

### Administration

The invention is typically intended for use with mammalian subjects, in particular human subjects. The combination treatment will typically be administered to a subject in need thereof, in particular a mammalian subject in need thereof, in particular a human subject in need thereof.

In a further aspect the invention provides a CTPS1 inhibitor and a BCL2 inhibitor for use in the treatment of cancer.

One aspect of the invention provides the use of a CTPS1 inhibitor in the manufacture of a medicament for the treatment of cancer with a BCL2 inhibitor.

A further aspect of the invention provides the use of a BCL2 inhibitor in the manufacture of a medicament for the treatment of cancer with a CTPS1 inhibitor.

A further aspect of the invention provides the use of a CTPS1 inhibitor and a BCL2 inhibitor in the manufacture of a medicament for the treatment of cancer.

A further aspect of the invention provides a method of treating cancer in a subject which method comprises administering to the subject a CTPS1 inhibitor and a BCL2 inhibitor.

A further aspect of the invention provides a pharmaceutical composition comprising a CTPS1 inhibitor and a BCL2 inhibitor, suitably for use in the treatment of cancer.

Suitably the CTPS1 inhibitor and the BCL2 inhibitor act synergistically in treating the cancer. The CTPS1 inhibitor and the BCL2 inhibitor act ‘synergistically’ if their combined administration results in a beneficial effect greater than the sum of the beneficial effects of each agent administered alone. Suitably the CTPS1 inhibitor and the BCL2 inhibitor act synergistically if they achieve a Bliss score (Bliss 1939; Zheng 2021) of equal to or greater than 10 when applied to a cancer cell line as set out in Example 6.

### Administration of the CTPS1 Inhibitor

The CTPS1 inhibitor may be administered by any suitable route, which may depend on the nature of the specific agent. Exemplary routes include oral, parenteral, buccal, sublingual, nasal or rectal administration. Conveniently, the CTPS1 inhibitor is administered orally.

The CTPS1 inhibitor may be provided in the form of a pharmaceutical composition comprising the CTPS1 inhibitor and a pharmaceutically acceptable carrier or excipient.

If delivered orally, the CTPS1 inhibitor may suitably be delivered in a solid pharmaceutical composition (such as a tablet, capsule or lozenge) or in a liquid pharmaceutical composition (such as a suspension, emulsion or solution).

A liquid formulation will generally consist of a suspension or solution of the CTPS1 inhibitor in a suitable liquid carrier e.g. an aqueous solvent such as water, ethanol or glycerine, or a non-aqueous solvent, such as polyethylene glycol or an oil. The formulation may also contain a suspending agent, preservative, flavouring and/or colouring agent.

A tablet formulation can be prepared using any suitable pharmaceutical carrier(s) routinely used for preparing solid formulations, such as magnesium stearate, starch, lactose, sucrose and cellulose.

Suitably, the pharmaceutical composition is in unit dose form, such as a tablet, capsule or ampoule. Suitably the unit dose form is for oral delivery.

The pharmaceutical composition may for example contain from 0.1% to 99.99% by weight, for example from 10 to 60% by weight, of the active material, depending on the method of administration. The pharmaceutical composition may contain from 0.01% to 99% by weight, for example 40% to 90% by weight, of the carrier, depending on the method of administration. The pharmaceutical composition may contain from 0.05 mg to 2000 mg of the active material, for example from 1.0 mg to 500 mg, depending on the method of administration. The pharmaceutical composition may contain from 50 mg to 1000 mg of the carrier, for example from 100 mg to 400 mg, depending on the method of administration.

The dose of the compound used will vary in the usual way with the seriousness of the cancer, the weight of the sufferer, and other similar factors. However, as a general guide, suitable unit doses may be 0.05 mg to 1000 mg, more suitably 1.0 mg to 500 mg, and such unit doses may be administered more than once a day, for example two or three a day. Such therapy may extend for a number of weeks, months or longer. A plurality of unit does, such as a plurality of tablets, may be taken together.

Suitably, the CTPS1 inhibitor is administered orally, such as administered orally in a solid pharmaceutical composition.

The dose provided to a subject will typically be a safe and effective dose, i.e. an amount providing an acceptable balance of desired benefits and undesired side effects. A “safe and effective amount” is intended to include an amount of a compound that is effective to achieve a desirable effect in treatment of a disease-state. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms. The safe and effective amount is one that is sufficient to achieve the desirable effect when the CTPS1 inhibitor is administered with the BCL2 inhibitor.

For avoidance of doubt, a “safe and effective amount” as recited herein can be achieved by any suitable dosage regimen. Hence, for example, references herein to administering a safe and effective amount of a compound, such as by a particular administration route, include achieving the safe and effective amount via a single dose or by plural doses, such as administered by the specified administration route. For instance, orally administering a safe and effective amount includes both orally administering a single dose and orally administering any plural number of doses, provided that a safe and effective amount is thereby achieved by oral administration.

### Administration of the BCL2 Inhibitor

The BCL2 inhibitor may be administered by any suitable route, which may depend on the nature of the specific agent. Exemplary routes include oral, parenteral, buccal, sublingual, nasal or rectal administration. Conveniently, the BCL2 inhibitor is administered orally. The BCL2 inhibitor may be provided in the form of a pharmaceutical composition comprising the BCL2 inhibitor and a pharmaceutically acceptable carrier or excipient.

If delivered orally, the BCL2 inhibitor may suitably be delivered in a solid pharmaceutical composition (such as a tablet, capsule or lozenge) or in a liquid pharmaceutical composition (such as a suspension, emulsion or solution).

A liquid formulation will generally consist of a suspension or solution of the BCL2 inhibitor in a suitable liquid carrier e.g. an aqueous solvent such as water, ethanol or glycerine, or a non-aqueous solvent, such as polyethylene glycol or an oil. The formulation may also contain a suspending agent, preservative, flavouring and/or colouring agent.

A tablet formulation can be prepared using any suitable pharmaceutical carrier(s) routinely used for preparing solid formulations, such as magnesium stearate, starch, lactose, sucrose and cellulose.

Suitably, the pharmaceutical composition is in unit dose form, such as a tablet, capsule or ampoule. Suitably the unit dose form is for oral delivery.

The pharmaceutical composition may for example contain from 0.1% to 99.99% by weight, for example from 10 to 60% by weight, of the active material, depending on the method of administration. The pharmaceutical composition may contain from 0.01% to 99% by weight, for example 40% to 90% by weight, of the carrier, depending on the method of administration. The pharmaceutical composition may contain from 0.05 mg to 2000 mg of the active material, for example from 1.0 mg to 500 mg, suitably 5 mg to 15 mg, such as 10 mg, depending on the method of administration. For oral administration, 10 mg may be desirable. The pharmaceutical composition may contain from 50 mg to 1000 mg, for example from 100 mg to 400 mg of the carrier, depending on the method of administration.

The dose of the compound used will vary in the usual way with the seriousness of the cancer, the weight of the sufferer, and other similar factors. However, as a general guide, suitable unit doses may be 0.05 mg to 1000 mg, more suitably 1.0 mg to 500 mg, and such unit doses may be administered more than once a day, for example two or three a day. Such therapy may extend for a number of weeks, months or longer. A plurality of unit does, such as a plurality of tablets, may be taken together.

In one embodiment the BCL2 inhibitor is administered daily for a period 5 weeks. The BCL2 inhibitor may be administered at a daily dose of 20 mg for week 1, 50 mg for week 2, 100 mg for week 3, 200 mg for week 4 and 400 mg for week 5.

The BCL2 inhibitor may be administered at a daily dose of up to 600 mg, suitably 1.0 mg to 500 mg.

Suitably, the BCL2 inhibitor is administered orally, such as administered orally in a solid pharmaceutical composition.

The dose provided to a subject will typically be a safe and effective dose, i.e. an amount providing an acceptable balance of desired benefits and undesired side effects. A “safe and effective amount” is intended to include an amount of a compound that is effective to achieve a desirable effect in treatment of a disease-state. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms. The safe and effective amount is one that is sufficient to achieve the desirable effect when the CTPS1 inhibitor is administered with the BCL2 inhibitor.

For avoidance of doubt, a “safe and effective amount” as recited herein can be achieved by any suitable dosage regimen. Hence, for example, references herein to administering a safe and effective amount of a compound, such as by a particular administration route, include achieving the safe and effective amount via a single dose or by plural doses, such as administered by the specified administration route. For instance, orally administering a safe and effective amount includes both orally administering a single dose and orally administering any plural number of doses, provided that a safe and effective amount is thereby achieved by oral administration.

### Administration Regimes

The CTPS1 inhibitor and BCL2 inhibitor may be administered separately, sequentially or simultaneously.

The CTPS1 inhibitor may be administered before the BCL2 inhibitor. Alternatively, the BCL2 inhibitor may be administered before the CTPS1 inhibitor.

To maintain therapeutic efficacy whilst controlling toxicity, the CTPS1 inhibitor and/or BCL2 inhibitor may be administered intermittently. Intermittently in this context means that the CTPS1 inhibitor and/or the BCL2 inhibitor are not administered every day of a treatment cycle (e.g. the CTPS1 inhibitor and/or the BCL2 inhibitor are administered for 4 days in each 7 day period of a treatment cycle). It will be understood that when the CTPS1 inhibitor and BCL2 inhibitor are both administered intermittently, they need not be administered according to the same schedule.

Suitably, the CTPS1 inhibitor and/or BCL2 inhibitor may be administered continuously i.e. administered at least daily in a treatment cycle (e.g. the CTPS1 inhibitor and/or the BCL2 inhibitor are administered each day of a treatment cycle).

Suitably, the CTPS1 inhibitor is administered intermittently and the BCL2 inhibitor is administered intermittently. Suitably, the CTPS1 inhibitor is administered continuously and the BCL2 inhibitor is administered continuously. Suitably, the CTPS1 inhibitor is administered intermittently and the BCL2 inhibitor is administered continuously. Suitably, the CTPS1 inhibitor is administered continuously and the BCL2 inhibitor is administered intermittently.

The CTPS1 inhibitor and the BCL2 inhibitor may be delivered in co-formulation (where compatible with co-formulation and whether the dosage regimes of the two agents allow) or in separate formulations. Most suitably the CTPS1 inhibitor and the BCL2 inhibitor are delivered in co-formulation or in separate formulations which are simultaneously administered. Alternatively, if delivered in separate formulations, the CTPS1 inhibitor and the BCL2 inhibitor may be delivered at different times.

If separately formulated, the CTPS1 inhibitor (or a pharmaceutical composition comprising such, such as a tablet or capsule) and BCL2 inhibitor (or a pharmaceutical composition comprising such, such as a tablet or capsule) may be provided in separate containers.

If separately formulated, the CTPS1 inhibitor and BCL2 inhibitor may be provided in the form of a kit of parts comprising:
a) a first container comprising a CTPS1 inhibitor; and
b) a second container comprising a BCL2 inhibitor.

More suitably, the CTPS1 inhibitor and BCL2 inhibitor may be provided in the form of a kit of parts comprising a first container comprising a CTPS1 inhibitor (or a pharmaceutical composition comprising such, such as a tablet or capsule) and a second container comprising a BCL2 inhibitor (or a pharmaceutical composition comprising such, such as a tablet or capsule).

Combinations with Further Agents

Treatment with the CTPS1 inhibitor and BCL2 inhibitor may be combined with one or more further pharmaceutically acceptable active ingredients, which may be selected from: anti-mitotic agents such as vinblastine, paclitaxel and docetaxel; alkylating agents, for example cisplatin, carboplatin, dacarbazine and cyclophosphamide; antimetabolites, for example 5-fluorouracil, cytosine arabinoside and hydroxyurea; intercalating agents for example adriamycin and bleomycin; topoisomerase inhibitors for example etoposide, topotecan and irinotecan; thymidylate synthase inhibitors for example raltitrexed; PI3 kinase inhibitors for example idelalisib; mTor inhibitors for example everolimus and temsirolimus; proteasome inhibitors for example bortezomib; histone deacetylase inhibitors for example panobinostat or vorinostat; and hedgehog pathway blockers such as vismodegib.

The CTPS1 inhibitor, BCL2 inhibitor and the additional pharmaceutically acceptable active ingredients may each be administered in any combination of separate, sequential or simultaneous dosing. If administered simultaneously, the CTPS1 inhibitor and BCL2 inhibitor may be e.g. (a) formulated together but separately from the further pharmaceutically acceptable active ingredient, (b) formulated separately from each other and separately from the further pharmaceutically acceptable active ingredient (c) formulated together with the further pharmaceutically acceptable active ingredient.

The CTPS1 inhibitor, the BCL2 inhibitor and the additional pharmaceutically acceptable active ingredients may each be administered in any combination of separate, sequential or simultaneous dosing. The CTPS1 inhibitor, BCL2 inhibitor and the additional pharmaceutically acceptable active ingredients may be e.g. (a) formulated together but separately from the further pharmaceutically acceptable active ingredient, (b) formulated separately from each other and separately from the further pharmaceutically acceptable active ingredient, (c) formulated together with the further pharmaceutically acceptable active ingredient; (d) formulated separately from each other, but one of the CTPS1 inhibitor or BCL2 inhibitor formulated together with the further pharmaceutically acceptable active ingredient.

The further pharmaceutically acceptable active ingredient may be selected from tyrosine kinase inhibitors such as, for example, axitinib, dasatinib, erlotinib, imatinib, nilotinib, pazopanib and sunitinib. Alternatively, the further pharmaceutically acceptable active ingredient may be selected from azacitidine, decitabine, or cytarabine.

Further pharmaceutically acceptable active ingredients also include anticancer antibodies, such as those selected from the group consisting of anti-CD20 antibodies (such as obinutuzumab, ofatumumab, tositumomab or rituximab) or other antibodies such as olaratumab, daratumumab, necitumumab, dinutuximab, traztuzumab emtansine, pertuzumab, brentuximab, panitumumab, catumaxomab, bevacizumab, cetuximab, traztuzumab and gentuzumab ozogamycin.

The CTPS1 inhibitor and BCL2 inhibitor may also be administered in combination with radiotherapy, surgery, hyperthermia therapy or cryotherapy.

### Cancer

Response to venetoclax has been associated with BCL2 expression (higher expression correlates with better sensitivity to venetoclax) and the ratio of BCL2 expression to the expression of BCL2L1 and MCL1 (higher BCL2:BCL2L1 and BCL2:MCL1 ratios correlate with better sensitivity to venetoclax) in haematological and solid tumours (Punnoose 2016; Tutusaus 2018). Mitochondrial priming has also been associated with response to venetoclax in chronic lymphocytic leukaemia, whereby cells that are primed for apoptosis are more sensitive to the effects of venetoclax (Anderson 2016).

Intersecting BCL2 expression with markers of replication stress may prioritise tumour types most likely to show sensitivity to combined inhibition of BCL2 and CTPS1. Among different cancer types, haematological tumours show the highest levels of both BCL2 expression and replication stress, and combined CTPS1 and BCL2 inhibition has been shown herein to be effective against B cell lymphoma and myeloma. Solid tumours showing high levels of both BCL2 expression and replicative stress are also expected to be indications for this combination. Combining measurement of BCL2 expression ratio [BCL2/(BCL2L1+MCL1)] and a marker of replication stress [pCHEK1] enables ranking of cancers by their predicted response to combined inhibition of CTPS1 and BCL2. Based on this, cancers particularly susceptible to combined CTPS1 and BCL2 inhibition may be as follows: leukemia, lymphoma, lung cancer, bone cancer, melanoma, prostate cancer, brain tumours, colorectal cancer, esophagogastric cancer, breast cancer, endometrial cancer renal cancer, sarcoma, hepatic cancer, pancreatic cancer, bladder cancer, thyroid cancer, ovarian cancer, head and neck cancer, mesothelioma and biliary tract cancer (data used for this analysis available at .nci.nih.gov/rsconnect/cellminercdb/ accessed February 2022). More suitably leukemia, lymphoma, lung cancer, bone cancer, melanoma, prostate cancer, brain tumours, colorectal cancer, esophagogastric cancer, breast cancer, endometrial cancer renal cancer, sarcoma, hepatic cancer and pancreatic cancer; more suitably leukemia, lymphoma, lung cancer, bone cancer, melanoma, prostate cancer, brain tumours and colorectal cancer.

In one embodiment the cancer is a cancer which may be susceptible to replication stress, displays high replicative stress. By a ‘high’ level it is meant that the cancer displays a level of replication stress which is higher than an average cancer. Suitably the cancer expresses BCL2.

In one embodiment the cancer is a cancer displaying high expression of BCL2. By ‘high’ expression it is meant that the cancer displays a level of expression which is higher than an average cancer.

In one embodiment the cancer is a cancer displaying a high BCL2:BCL2L1 ratio and/or a high BCL2:MCL1 ratio. By a ‘high’ ratio it is meant that the cancer displays a ratio which is higher than an average cancer.

Suitably the cancer is a haematological cancer, such as acute myeloid leukemia, angioimmunoblastic T-cell lymphoma, B-cell acute lymphoblastic leukemia, Sweet syndrome, T-cell non-Hodgkin lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell non-Hodgkin lymphoma (including Burkitt lymphoma, diffuse large B-cell lymphoma, follicular lymphoma, mantle cell lymphoma, marginal zone lymphoma), hairy cell leukemia, Hodgkin lymphoma, lymphoblastic lymphoma, lymphoplasmacytic lymphoma, mucosa-associated lymphoid tissue lymphoma, multiple myeloma, myelodysplastic syndrome, plasma cell myeloma, primary mediastinal large B-cell lymphoma, chronic myeloproliferative disorders (such as chronic myeloid leukemia, primary myelofibrosis, essential thrombocythemia, polycythemia vera) or chronic lymphocytic leukemia. Most suitably, T cell lymphoma, diffuse large B cell lymphoma, plasma cell myeloma, acute myeloid leukaemia, chronic lymphocytic leukaemia or peripheral T cell lymphoma. A further haematological cancer of interest is T-cell prolymphocytic leukemia. Other haematological cancers of interest are myelodysplastic syndromes (MDS), such as MDS with single lineage dysplasia, MDS with multilineage dysplasia or MDS with excess blasts.

Alternatively, the cancer is a non-haematological cancer, such as selected from the group consisting of lung cancer, bone cancer, melanoma, prostate cancer, brain tumours, colorectal cancer, esophagogastric cancer, breast cancer, endometrial cancer renal cancer, sarcoma, hepatic cancer, pancreatic cancer, bladder cancer, thyroid cancer, ovarian cancer, head and neck cancer, mesothelioma and biliary tract cancer.

The CTPS1 inhibitor, BCL2 inhibitor, use, method, composition or kit of the invention may be for administration to a subject identified as having a cancer expected to be susceptible to treatment by a CTPS1 inhibitor and a BCL2 inhibitor. The CTPS1 inhibitor, BCL2 inhibitor, use, method, composition or kit of the invention may be for administration to a subject from whom a sample of cancer cells has been shown to be susceptible to treatment by a CTPS1 inhibitor and a BCL2 inhibitor. A ‘susceptible’ cancer or cancer cell sample in this context is one which is associated with generally demonstrating a benefit from the treatment according to the invention relative to treatment with CTPS1 or BCL2 inhibitors alone, e.g. additive or, suitably, synergistic effects—high in vivo efficacy, reduction in the dose required for effect in vivo and/or an improved safety profile/reduced side effects.

The invention is further exemplified by the following non-limiting examples.

### EXAMPLES

### Example 1: Human CTPS1 Enzyme Inhibition

### Enzyme Inhibitory Activities of Compounds CTPS1-IA and CTPS1-IB

The enzyme inhibitory activities of compounds CTPS1-IA and CTPS1-IB against CTPS1 were determined using the ADP-Glo™ Max assay (Promega, UK).

CTPS1-IA is 4-(2-(cyclopropanesulfonamido)pyrimidin-4-yl)-N-(5-(6-ethoxypyrazin-2-yl)pyridin-2-yl)tetrahydro-2H-pyran-4-carboxamide.
CTPS-IB is N-(5-(6-ethoxypyrazin-2-yl)pyridin-2-yl)-4-(2-(ethylsulfonamido)pyrimidin-4-yl)tetrahydro-2H-pyran-4-carboxamide.

Assays for human CTPS1 were performed in 1× assay buffer containing 50 mM Tris, 10 mM MgCl₂, 0.01% Tween-20, pH to 8.0 accordingly. Finally, immediately before use, L-cysteine was added to the 1× assay buffer to a final concentration of 2 mM. All reagents are from Sigma-Aldrich unless specified otherwise. Human full length active C-terminal FLAG-His₈-tag CTPS1 (UniProtKB—P17812, CTPS[1-591]-GGDYKDDDDKGGHHHHHHHH, SEQ ID NO: 1) was obtained from Proteros biostructures GmbH.

### Assay Procedure

3× human CTPS1 protein was prepared in 1× assay buffer to the final working protein concentration required for the reaction. A 2 uL volume per well of 3× human CTPS1 protein was mixed with 2 uL per well of 3× test compound (compound prepared in 1× assay buffer to an appropriate final 3× compound concentration respective to the concentration response curve designed for the compounds under test) for 10 minutes at 25° C. The enzymatic reaction was then initiated by addition of a 2 uL per well volume of a pre-mixed substrate mix (UltraPure ATP from ADP-Glo™ Max kit (0.31 mM), GTP (0.034 mM), UTP (0.48 mM) and L-glutamine (0.186 mM)) and the mixture was incubated for an appropriate amount of time within the determined linear phase of the reaction at 25° C. under sealed plate conditions with constant agitation at 500 revolutions per minute (rpm). ADP-Glo™ Max reagent was added for 60 minutes (6 μL per well) and subsequently ADP-Go™ Max development reagent was added for 60 minutes (12 uL per well) prior to signal detection in a microplate reader (EnVision® Multilabel Reader, Perkin Elmer). Following each reagent addition over the course of the assay, assay plates were pulse centrifuged for 30 seconds at 500 rpm.

In all cases, the enzyme converts ATP to ADP and the ADP-Glo™ Max reagent subsequently depletes any remaining endogenous ATP in the reaction system. The ADP-Glo™ Max detection reagent converts the ADP that has been enzymatically produced back into ATP and using ATP as a substrate together with luciferin for the enzyme luciferase, light is generated which produces a detectable luminescence. The luminescent signal measured is directly proportional to the amount of ADP produced by the enzyme reaction and a reduction in this signal upon compound treatment demonstrates enzyme inhibition. The percentage inhibition produced by each concentration of compound was calculated using the equation shown below:

$$\% Inhibition = 1 - \frac{( {Mean}_{Min} - {Mean}_{Inh} )}{( {Mean}_{Min} - {Mean}_{Max )}} \times 100$$

Percentage inhibition was then plotted against compound concentration, and the 50% inhibitory concentration (IC₅₀) was determined from the resultant concentration-response curve. The data for the tested compounds are presented below.

TABLE 1
Human CTPS1 Enzyme Inhibition data

| Compound | IC₅₀ (um) |
|---|---|
| CTPS1-IA | ≤0.1 |
| CTPS1-IB | ≤0.1 |

Both compounds were found to demonstrate inhibition of CTPS1 enzyme in this assay. Consequently, these compounds may be expected to have utility in the inhibition of CTPS1.

### Example 2: RapidFire/MS-based CTPS1 Enzyme Selectivity Assays

### Human CTPS1 Versus CTPS2 Selectivity Assessment by RapidFire/MS Analysis.

The enzyme inhibitory activities against each target isoform of interest were determined for compounds using an optimised RapidFire high-throughput mass spectrometry (RF/MS) assay format. RF/MS assays for both human CTPS1 and CTPS2 were performed in assay buffer consisting of 50 mM HEPES (Merck), 20 mM MgCl₂, 5 mM KCl, 1 mM DTT, 0.01% Tween-20, pH to 8.0 accordingly. Human full-length active C-terminal FLAG-His-tag CTPS1 (UniProtKB—P17812, CTPS[1-591]-GGDYKDDDDKGGHHHHHHHH, SEQ ID NO: 1) was obtained from Proteros biostructures GmbH. Human full length active C-terminal FLAG-His-Avi tagged CTPS2 (UniProtKB—Q9NRF8, CTPS2 [1-586]-DYKDDDDKHHHHHHGLNDIFEAQKIEWHE, SEQ ID NO: 2) was obtained from Harker Bio.

### Assay Procedure

Human CTPS (1 or 2) protein was prepared in 1× assay buffer to the final working protein concentration required for the reaction. A 2 uL volume per well of 2×CTPS (1 or 2) protein was mixed with 40 nL of compound using acoustic (ECHO) delivery and incubated for 10 minutes at 25° C. Each isoform enzymatic reaction was subsequently initiated by addition of 2 uL per well of a 2× substrate mix in assay buffer. For hCTPS1: ATP (0.3 mM), UTP (0.2 mM), GTP (0.07 mM) and L-glutamine (0.1 mM). For hCTPS2: ATP (0.1 mM), UTP (0.04 mM), GTP (0.03 mM) and L-glutamine (0.1 mM). Each mixture was incubated for an appropriate amount of time per isoform within the determined linear phase of the reaction at 25° C. A 60 uL volume of stop solution (1% formic acid with 0.5 uM ¹³C₉-¹⁵N₃-CTP in H₂O) was added and the plate immediately heat-sealed and centrifuged for 10 minutes at 4,000 rpm. Following centrifugation, plates were loaded onto the Agilent RapidFire microfluidic solid phase extraction system coupled to an AP14000 triple quadrupole mass spectrometer (RF/MS) for analysis.

In all cases, the enzyme converts UTP to CTP. Highly specific and sensitive multiple reaction monitoring (MRM) MS methods may be optimised for the detection of the enzymatic reaction product, CTP, and the stable isotope labelled product standard ¹³C₉-¹⁵N₃-CTP. Readout for data analysis was calculated as the ratio between the peak area of the product CTP and the internal standard ¹³C₉-¹⁵N₃-CTP. For data reporting, the following equation was used:

$$R = \frac{P}{IS}$$

(R=ratio/readout, P=product signal area, IS=internal standard signal area)

For each screening plate, the means of the negative (DMSO) and positive control values were used for the calculation of the respective assay window (S/B) and Z′ values. The median of the respective control values was used for calculation of percent inhibition according to the following equation:

$$I = \frac{{R}_{neg} - {R}_{sample}}{[ {R}_{n e g} - {R}_{p o s} ]} \%$$

(I=Inhibition, R_{neg}=median of negative control readout values, Rₚₒₛ=median of positive control readout values, Rₛₐₘₚₗₑ=sample readout value)

Percentage inhibition was then plotted against compound concentration, and the 50% inhibitory concentration (IC₅₀) was determined from the resultant concentration-response curve.

Fold selectivity between CTPS1 and CTPS2 was subsequently calculated according to the following equation:

$$Fold selectivity = \frac{CTPS 2 {IC}_{50}}{CTOS 1 {IC}_{50}}$$

CTPS1-IA and CTPS1-IB was tested for selectivity. The results are presented below.

TABLE 2
Selectivity data

| Compound | Selectivity |
|---|---|
| CTPS1-IA | >60 fold |
| CTPS1-IB | >60 fold |

These compounds may be expected to have utility in the treatment of diseases whereby a selective CTPS1 compound is beneficial.

### Example 3: Human BCL2 Inhibition

Inhibition of BCL2 protein by a small molecule inhibitor can be measured by the ability of the small molecule inhibitor to disrupt the interaction between BCL2 and its downstream protein target, BAD. In this assay, recombinant BCL2 protein at known concentration is incubated with a fluorescent labelled peptide from a BH3 protein that is a known binding partner of BCL2 at known concentration. The fluorescent labelled BH3 peptide binds strongly to BCL2 but can be displaced by a small molecular inhibitor of BCL2. The amount of free fluorescent labelled BH3 peptide, which can be accurately quantitated following excitation at a suitable wavelength and measurement of the resulting fluorescence, can then be used to determine the binding efficiency of the small molecular inhibitor, by measuring fluorescence across a range of concentrations of the small molecular inhibitor. This method is disclosed in Zhang 2002 which is incorporated herein by reference in its entirety for the purpose of methods of measuring BCL2 inhibition.

### Example 4: BCL2 Selectivity

The method outlined in Example 3 can be adapted to replace recombinant human BCL2 protein with either recombinant human MCL1 or recombinant human BCL2L1 protein, in order to measure the inhibition of MCL1 and BCL2L1. The relative inhibition of BCL2 compared with BCL2L1 and MCL1 informs the selectivity of the small molecule inhibitor for BCL2. This method is disclosed in Zhang 2002 which is incorporated herein by reference in its entirety for the purpose of methods of measuring selectivity of BCL2 inhibition.

### Example 5: CTPS1 Involvement in the Proliferation of Cancer Cells

Pathways involved in providing the key building blocks for nucleic acid replication are the purine and pyrimidine synthesis pathways, and pyrimidine biosynthesis has been observed to be up-regulated in tumors and neoplastic cells. CTPS activity is upregulated in a range of tumour types of both haematological and non-haematological origin, although heterogeneity is observed among patients. Linkages have also been made between high enzyme levels and resistance to chemotherapeutic agents.

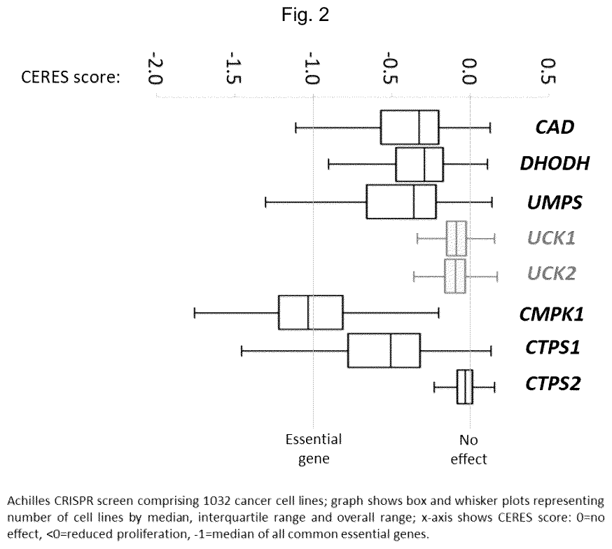

In an analysis of published data, CTPS1 was found by the present inventors to be essential for the proliferation of human cancer cells derived from a broad range of haematological and solid tumour types, whereas CTPS2 was invariably redundant. This analysis used data from the Achilles project where every gene in the human genome was independently deleted using CRIPR technology in each of 324 human cancer cell lines, and the effects of each gene deletion was assessed using an in vitro proliferation assay (Behan 2019). This dataset has subsequently been expanded to include data from 1,032 human cancer cell lines (Cancer Dependency Map:p.org/). The effects of deletion of different genes in the pyrimidine synthesis pathway were assessed (see FIG. 2). Deletion of CTPS2 had no effect on cancer cell proliferation. Deletion of genes in the salvage pathway (UCK1, UCK2) had minimal effect on cell proliferation. Deletion of CMPK1 had a marked effects on cell proliferation, consistent with CMPK1 being an essential gene. Deletion of CTPS1, UMPS, DHODH or CAD inhibited cancer cell proliferation with an effect that is consistent with dependency of cancer cells on the products of these genes; inhibition of CTPS1 produced the greatest impairment of cancer cell proliferation. These findings indicate that the majority of cancer cells are dependent on CTPS1 for cell proliferation, whereas CTPS2 is not required.

In recent work, the CTPS1 isoform has shown higher enzymatic activity than CTPS2. Taken together with the CRISPR study analysis outlined above, these findings highlight CTPS1 as the more potent CTP synthase enzyme and identify a non-redundant role for CTPS1 in the proliferation of human cancer cells, thus identifying CTPS1 as a potential therapeutic target in a wide range of human malignancies.

### Example 6: Effect of Combined Treatment with a CTPS1 Inhibitor and a BCL2 Inhibitor

In vitro proliferation assays were performed using human cancer cell lines to look for interactions between the antiproliferative effects of CTPS1-IA and (a) venetoclax or (b) BCL201.

Cell lines (available from commercial repositories, such as Deutsche Sammlung von Mikroorganismen und Zellkulturen) were plated in triplicate at a density of 0.2×10⁶ cells/ml in 96 well plates. CTPS1-IA and venetoclax were added at prespecified concentrations that were specific to each cell line, covering concentrations above and below the IC₅₀ value for the individual agents, and viability was assessed after 72 hours incubation using a tetrazolium salt-based colourimetric assay. CTPS1-IA and venetoclax were tested in a 4×4 matrix (total 16 conditions). Each compound was included at concentrations producing single agent 72 hour viability of 80-90%, 50-60% and 30-40%, as well as a no drug condition.

Analogous assays were carried out using BCL201 in place of venetoclax.

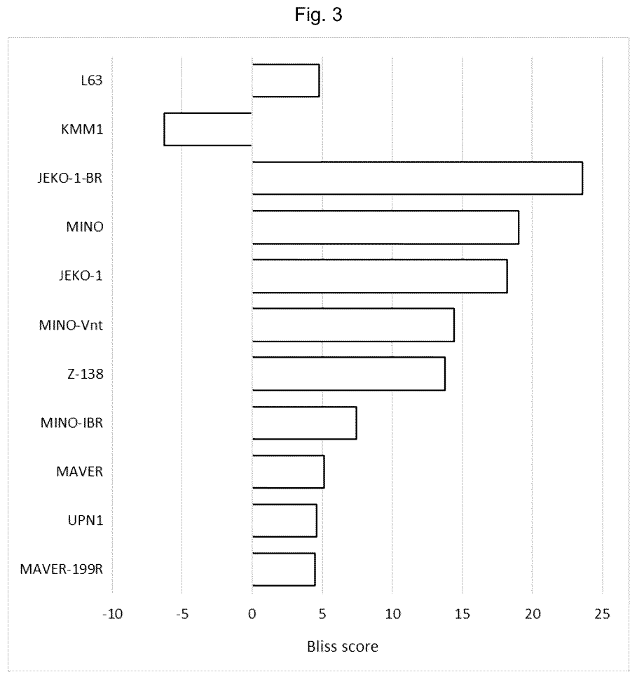

The bar chart in FIG. 3 shows Bliss scores (Bliss 1939; Zheng 2021) for the interaction between CTPS1-IA and venetoclax for a range of cell lines derived from B cell malignancies. A value of −10 to <10 indicates an additive effect and a value of >10 indicates synergy. Synergy between CTPS1-IA and venetoclax was observed for a subset of mantle cell lymphoma and myeloma cell lines. Additive effects were observed in other cell lines tested.

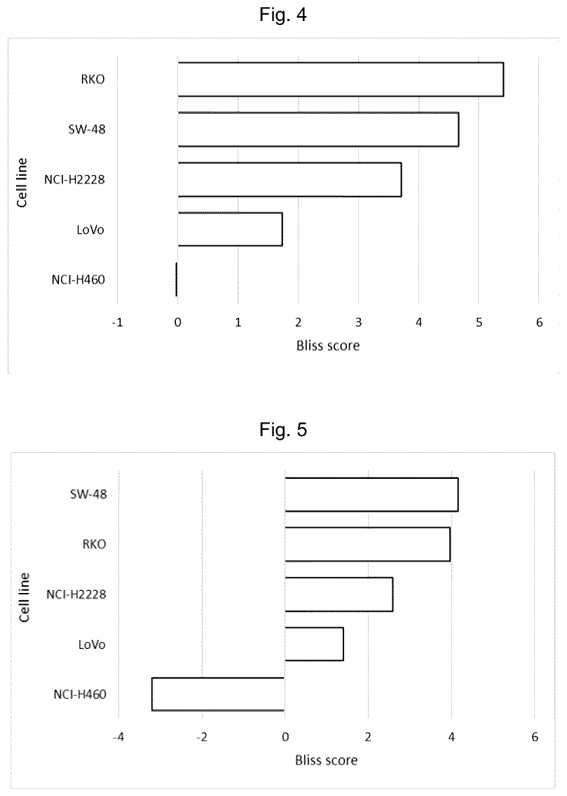

The bar charts in FIGS. 4 and 5 show Bliss scores for the interaction between CTPS1-IA and venetoclax and between CTPS1-IA and BCL201, respectively, for cell lines derived from solid tumours, either colorectal or lung cancer. A value of −10 to <10 indicates an additive effect and a value of >10 indicates synergy. Synergy was not observed between venetoclax or BCL201 for any of the cell lines tested.

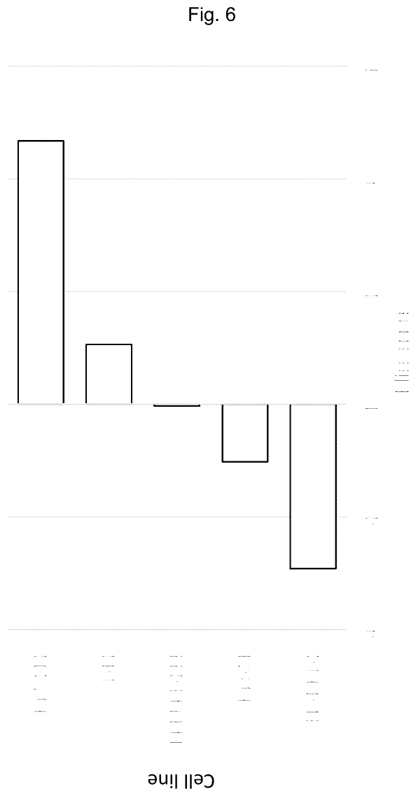

The bar chart in FIG. 6 shows Bliss scores for the interaction between CTPS1-IA and venetoclax for cell lines derived from T cell lymphoma. A value of −10 to <10 indicates an additive effect and a value of >10 indicates synergy. Synergy was not observed between CTPS1-IA and venetoclax for any of the T cell lymphoma cell lines tested.

### Example 7: In Vivo Effect of Combined Treatment with a CTPS1 Inhibitor and a BCL2 Inhibitor

In vivo tumour growth studies were performed using a human cancer cell line to look for interactions between the antiproliferative effects of CTPS1-IA and venetoclax.

Human mantle cell lymphoma cells (Z138) were transplanted subcutaneously into immunodeficient mice (NOD.Cg-Prkdc^{scid} Il2rg^{tm1Wjl}/SzJ (NSG) strain). Treatment was initiated when the tumours reached approximately 50 mm³; n=5 mice per group. CTPS1-IA was dosed at 30 mg/kg/day subcutaneously days 1-4 of a 7 day cycle; venetoclax was dosed at 75 mg/kg/day orally days 2-5 of a 7 day cycle for three cycles. Tumor size was assessed by calipers.

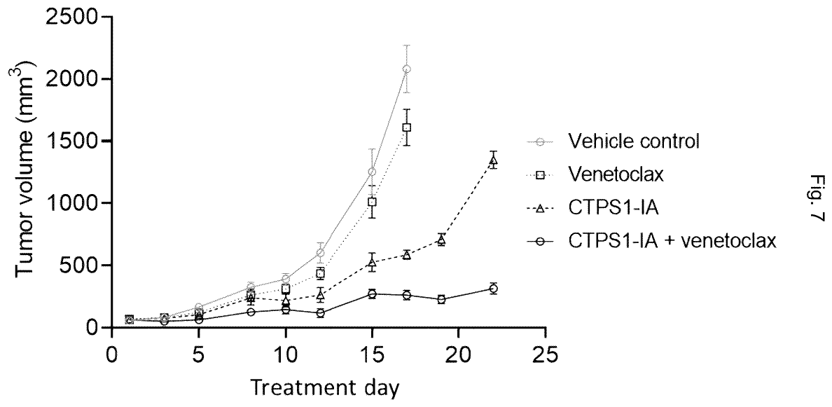

The graph in FIG. 7 shows the tumour growth in the treated and control mice, demonstrating in vivo synergy between CTPS1-IA and venetoclax.

### Example 8: RNA Transcript and Protein Levels Following Exposure to a CTPS1 Inhibitor

### RNA Sequencing

RNA was obtained from cancer cell lines treated with CTPS1-IA at IC50 or control for 24 hours. Libraries were prepared from 10 ng total RNA. mRNA poly(A) tails were tagged with universal adapters, sample-specific barcodes and unique molecular identifiers using template switching reverse transcriptase. Barcoded cDNAs from multiple samples were then pooled, amplified and tagmented using a transposon-fragmentation approach which enriches for 3′ ends of cDNA. A library of 350-800 bp length was run on an Illumina NovaSeq 6000 using NovaSeq 6000 SP Reagent Kit 100 cycles (ref #20027464). Raw fastq pairs used for analysis matched the following criteria: the 16 bases of the first read correspond to 6 bases for a sample-specific barcode and 10 bases for a unique molecular identifier. The second read (58 bases) corresponds to the captured poly(A) RNAs sequence. Demultiplexing of these fastq pairs was performed to generate one single-end fastq for each of the 96 samples. fastq files were then aligned with bwa to reference mRNA sequences and the mitochondrial genomic sequence (UCSC). DGE profiles were generated by parsing the alignment files (.bam) and counting for each sample the number of unique molecular identifiers associated with each RefSeq gene. Reads aligned on multiple genes, containing more than one mismatch with the reference sequence, or containing a polyA pattern were discarded. Expression values, corresponding to the absolute abundance of mRNAs in all samples, were used for further analysis. DESeq2 was used to normalize expression. Normalized counts were transformed with the vst (variance stabilized transformation) function from the DESeq library. Batch effects were corrected with the limma library function removeBatchEffect.

### Western Blot

Cells were incubated on ice for 40 minutes with lysis buffer then centrifuged at 11000 rpm for 30′. Total protein extracts were collected from supernatants and protein concentrations determined using a colorimetric assay. 30 μg of proteins were separated by SDS-PAGE using precast gels and transferred to membranes using the Trans-Blot Turbo system. Membranes were blocked for 1 hour in a 5% dry milk, TBS-Tween20 solution and incubated with primary antibodies overnight at 4° C. Membranes were incubated for at least 1 hour with secondary antibodies, either goat anti-mouse or goat anti-rabbit conjugated with peroxidase. Chemiluminescence was determined using the Clarity Western ECL Substrate on the Fusion FX imaging system.

### Results

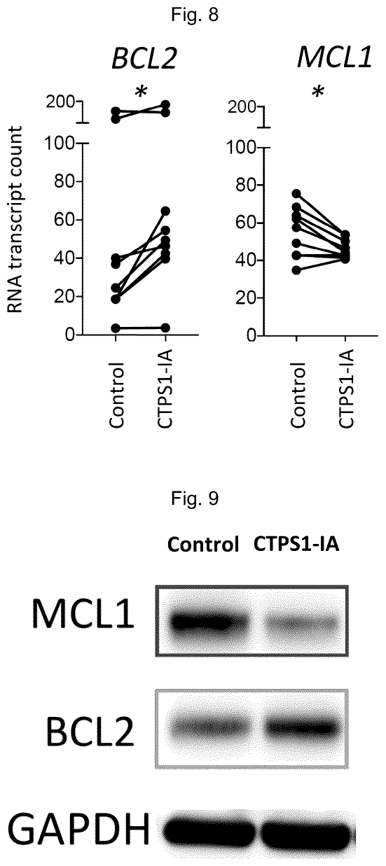

Exposure of human mantle cell lymphoma cell lines to CTPS1-IA resulted in an increase in BCL2 transcripts and a decrease in MCL1 transcripts (FIG. 8). These changes were confirmed at the protein level in an analysis of one of the cell lines (FIG. 9). Higher levels of BCL2 and a higher BCL2 to MCL1 ratio have both been associated with an increased likelihood of response to BCL2 inhibition with drugs such as venetoclax. These findings suggest that in some human cancer cells, exposure to CTPS1-IA sensitizes cells to the anti-tumour effects of venetoclax.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### Clauses of the Invention

A series of clauses setting out embodiments of the invention are as follows.
Clause 1. A CTPS1 inhibitor for use in the treatment of cancer with a BCL2 inhibitor.
Clause 2. A BCL2 inhibitor for use in the treatment of cancer with a CTPS1 inhibitor.
Clause 3. A CTPS1 inhibitor and a BCL2 inhibitor for use in the treatment of cancer.
Clause 4. Use of a CTPS1 inhibitor in the manufacture of a medicament for the treatment of cancer with a BCL2 inhibitor.
Clause 5. Use of a BCL2 inhibitor in the manufacture of a medicament for the treatment of cancer with a CTPS1 inhibitor.
Clause 6. Use of a CTPS1 inhibitor and a BCL2 inhibitor in the manufacture of a medicament for the treatment of cancer.
Clause 7. A method of treating cancer in a subject which method comprises administering to the subject a CTPS1 inhibitor and a BCL2 inhibitor.
Clause 8. A pharmaceutical composition comprising a CTPS1 inhibitor and a BCL2 inhibitor.
Clause 9. A kit of parts comprising:
a) a first container comprising a CTPS1 inhibitor; and
b) a second container comprising a BCL2 inhibitor.
   Clause 10. The CTPS1 inhibitor, BCL2 inhibitor, use, method, composition or kit according to any one of clauses 1 to 9, wherein the CTPS1 inhibitor has an IC₅₀ of 10 uM or lower in respect of human CTPS1 enzyme.
   Clause 11. The CTPS1 inhibitor, BCL2 inhibitor, use, method, composition or kit according to clause 10, wherein the CTPS1 inhibitor has an IC₅₀ of 1 uM or lower in respect of human CTPS1 enzyme.
   Clause 12. The CTPS1 inhibitor, BCL2 inhibitor, use, method, composition or kit according to clause 11, wherein the CTPS1 inhibitor has an IC₅₀ of 100 nM or lower in respect of human CTPS1 enzyme.
   Clause 13. The CTPS1 inhibitor, BCL2 inhibitor, use, method, composition or kit according to any one of clauses 10 to 12, wherein the IC₅₀ of the CTPS1 inhibitor is established using the assay procedure set out in Example 1.
   Clause 14. The CTPS1 inhibitor, BCL2 inhibitor, use, method, composition or kit according to any one of clauses 1 to 13, wherein the CTPS1 inhibitor has a selectivity for human CTPS1 over human CTPS2 of at least 2-fold.
   Clause 15. The CTPS1 inhibitor, BCL2 inhibitor, use, method, composition or kit according to clause 14, wherein the CTPS1 inhibitor has a selectivity for human CTPS1 over human CTPS2 of at least 30-fold.
   Clause 16. The CTPS1 inhibitor, BCL2 inhibitor, use, method, composition or kit according to clause 15, wherein the CTPS1 inhibitor has a selectivity for human CTPS1 over human CTPS2 of at least 60-fold, such as at least 1000-fold.
   Clause 17. The CTPS1 inhibitor, BCL2 inhibitor, use, method, composition or kit according to any one of clauses 14 to 16, wherein the selectivity of the CTPS1 inhibitor is established using the assay procedure set out in Example 2.
   Clause 18. The CTPS1 inhibitor, BCL2 inhibitor, use, method, composition or kit according to any one of clauses 1 to 17, wherein the CTPS1 inhibitor is a compound of formula (I)

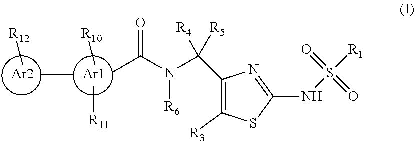

wherein
R¹ is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R₃ is H, CH₃, halo, OC₁₋₂alkyl or CF₃;
R₄ and R₅ are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R₄ and R₅ together with the carbon atom to which they are attached form a C₃₋₆ cycloalkyl or C₃₋₆heterocycloalkyl ring;
R₆ is H or C₁₋₃alkyl;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, Cl, CH₃, ethyl, OCH₃, CF₃, OCF₃ or CN;
R₁₂ is attached to Ar2 in the meta or ortho position relative to Ar1 and R₁₂ is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR₂₃R₂₄, SO₂CH₃, C(O)N(CH₃)₂, NHC(O)C₁₋₃alkyl, or a C₃₋₆heterocycloalkyl comprising one nitrogen located at the point of attachment to Ar2, or R₁₂ together with a nitrogen atom to which it is attached forms an N-oxide (N⁺—O⁻);
R₂₃ is H or C₁₋₂alkyl;
R₂₄ is H or C₁₋₂alkyl;
   or a pharmaceutically acceptable salt and/or pharmaceutically acceptable solvate thereof.
   Clause 19. The CTPS1 inhibitor, BCL2 inhibitor, use, method, composition or kit according to clause 18, wherein the CTPS1 inhibitor is selected from the compounds disclosed in List A or a pharmaceutically acceptable salt and/or pharmaceutically acceptable solvate thereof.
   Clause 20. The CTPS1 inhibitor, BCL2 inhibitor, use, method, composition or kit according to any one of clauses 1 to 17, wherein the CTPS1 inhibitor is a compound of formula (II):

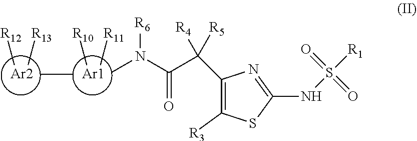

wherein
R₁ is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R₃ is H, halo, CH₃, OC₁₋₂alkyl or CF₃;
   or R₃ together with R₅ forms a 5- or 6-membered cycloalkyl or 5 or 6 membered oxygen-containing heterocycloalkyl;
R₄ and R₅ are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃ alkyl, C₁₋₆alkylOH, C₁₋₆haloalkyl, OC₁₋₆haloalkyl or NR₂₁R₂₂,
   or R₄ is H and R₅ together with R₃ form a 5- or 6-membered cycloalkyl or 5 or 6 membered oxygen-containing heterocycloalkyl,
   or R₄ and R₅ together with the carbon atom to which they are attached form a C₃₋₆cycloalkyl or C₃₋₆heterocycloalkyl,
   or R₄ is H and R₅ and R₆ are a C₂₋₃alkylene chain forming a 5- or 6-membered ring;
   or R₄ is O and R₅ is absent;
R₆ is H or C₁₋₃alkyl,
   or R₆ together with R₁₁ when in the ortho-position to the amide are a C₂alkylene chain forming a 5-membered ring,
   or R₅ and R₆ are a C₂₋₃alkylene chain forming a 5- or 6-membered ring and R₄ is H;
Ar1 is 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, Cl, CH₃, ethyl, OCH₃, CF₃, OCF₃ or CN,
   or R₁₁, when in the ortho-position to the amide, together with R₆ are a C₂alkylene chain forming a 5-membered ring;
R₁₂ is attached to Ar2 in the ortho or meta position relative to Ar1 and R₁₂ is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR₂₃R₂₄, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H, halo, CH₃ or OCH₃;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
or a pharmaceutically acceptable salt and/or pharmaceutically acceptable solvate thereof.
   Clause 21. The CTPS1 inhibitor, BCL2 inhibitor, use, method, composition or kit according to clause 20, wherein the CTPS1 inhibitor is selected from the compounds disclosed in List B or a pharmaceutically acceptable salt and/or pharmaceutically acceptable solvate thereof.
   Clause 22. The CTPS1 inhibitor, BCL2 inhibitor, use, method, composition or kit according to any one of clauses 1 to 17, wherein the CTPS1 inhibitor is a compound of formula (III):

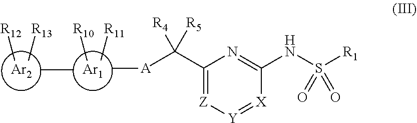

wherein
A is an amide linker having the following structure: —C(═O)NH— or —NHC(═O)—;
X is N or CH;
Y is N or CR₂;
Z is N or CR₃,
   with the proviso that when at least one of X or Z is N, Y cannot be N;
R₁ is C₁₋₅alkyl, C₀₋₂alkyleneC₃₋₅cycloalkyl which cycloalkyl is optionally substituted by CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl; and
   when A is —NHC(═O)—;
   R₄ and R₅ may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN; R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—;
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
   or a pharmaceutically acceptable salt and/or pharmaceutically acceptable solvate thereof.
   Clause 23. The CTPS1 inhibitor, BCL2 inhibitor, use, method, composition or kit according to clause 22, wherein the CTPS1 inhibitor is selected from the compounds disclosed in List C or a pharmaceutically acceptable salt and/or pharmaceutically acceptable solvate thereof.
   Clause 24. The CTPS1 inhibitor, BCL2 inhibitor, use, method, composition or kit according to clause 22, wherein the CTPS1 inhibitor is a compound of formula (IV):

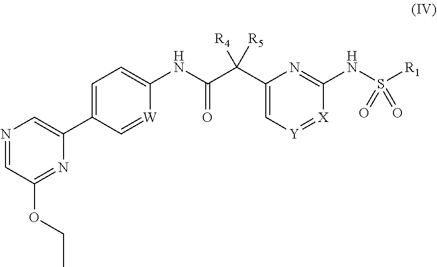

wherein:
(a) when R₄, R₅, X, Y and R₁ are as follows:

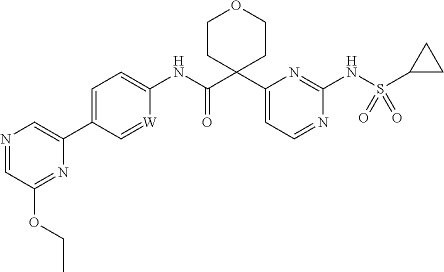

then W is N, CH or CF;
(b) when R₄, R₅, X, W and R₁ are as follows:

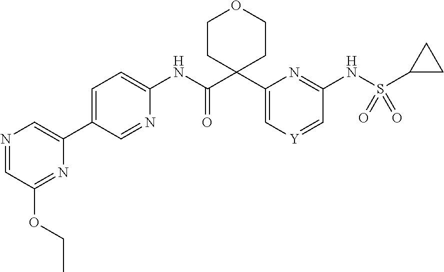

then Y is CH or N;
(c) when W, X, Y and R₁ are as follows:

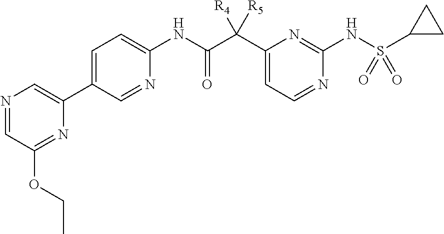

then R₄ and R₅ are joined to form the following structures:

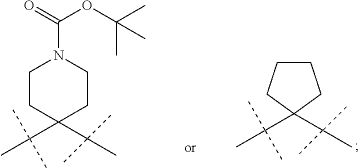

(d) when W, R₄, R₅, X and Y are as follows:

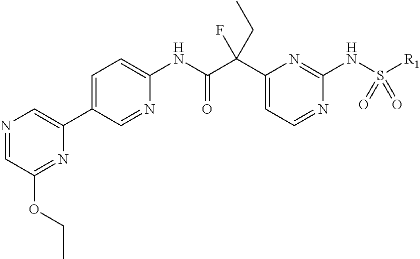

then R₁ is methyl or cyclopropyl; and
(e) the compound is selected from the group consisting of:

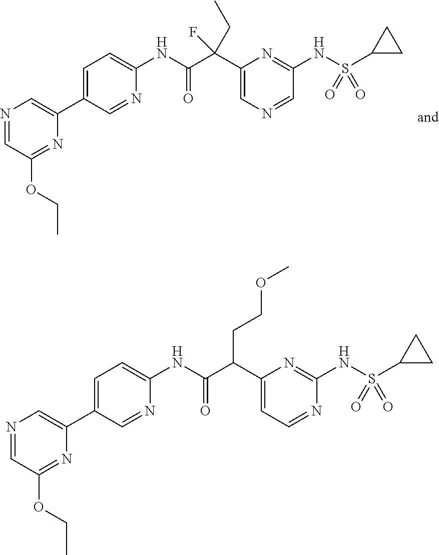 [SMILES: CCOc1cncc(-c2ccc(NC(=O)C(F)(CC)c3cncc(NS(=O)(=O)C4CC4)n3)nc2)n1 ; CCOc1cncc(-c2ccc(NC(=O)C(CCOC)c3ccnc(NS(=O)(=O)C4CC4)n3)nc2)n1]

or a pharmaceutically acceptable salt and/or pharmaceutically acceptable solvate thereof.
Clause 25. The CTPS1 inhibitor, BCL2 inhibitor, use, method, composition or kit according to clause 24, wherein the CTPS1 inhibitor is selected from the compounds disclosed in List D or a pharmaceutically acceptable salt and/or pharmaceutically acceptable solvate thereof.
Clause 26. The CTPS1 inhibitor, BCL2 inhibitor, use, method, composition or kit according to clause 22, wherein the CTPS1 inhibitor is a compound of formula (V):

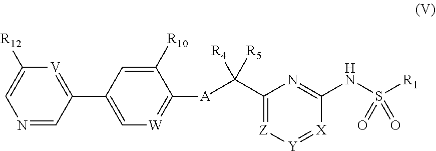

(a) when A, V, W, X, Y, Z, R₁, R₁₀ and R₁₂ are as follows:

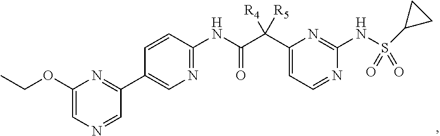

then R₄ and R₅ together with the carbon atom to which they attached form:

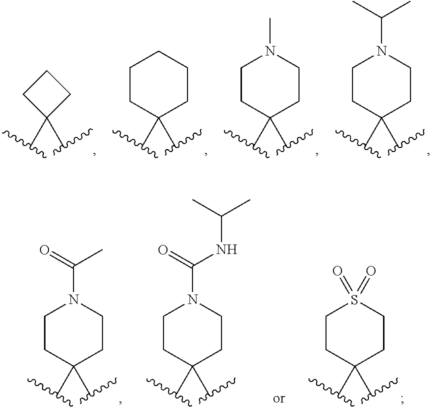

or
(b) when A, V, W, X, Y, Z, R₁, R₁₀ and R₁₂ are as follows:

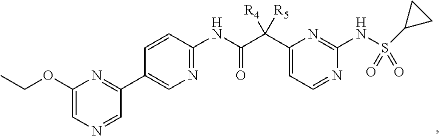

then R₄ and R₅ together with the carbon atom to which they are attached form:

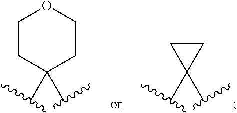 [SMILES: *C1(*)CCOCC1 ; *C1(*)CC1]

or
(c) when A, V, W, X, Y, Z, R₄, R₅, R₁₀ and R₁₂ are as follows:

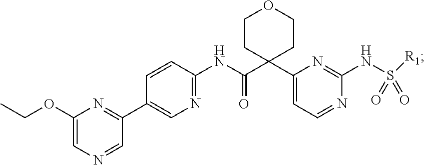

then R, is

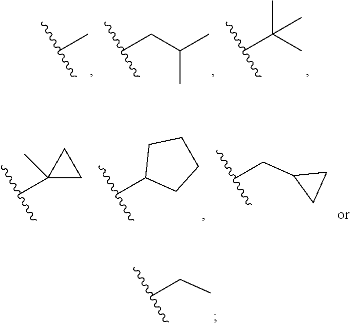

or
(d) when A, V, W, X, Y, Z, R₄, R₅, R₁₀ and R₁₂ are as follows:

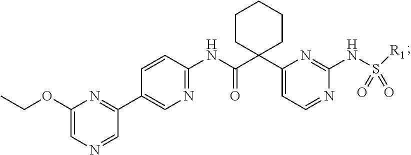

then R₁ is

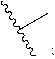

or
(e) when A, X, Y, Z, R₁, R₄ and R₅ are as follows:

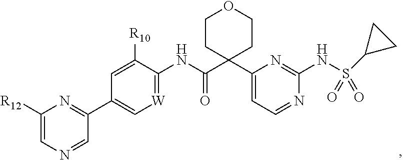

then V, W, R₁₀ and R₁₂ are:

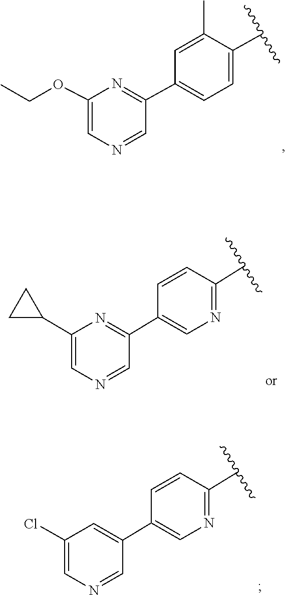 [SMILES: *c1ccc(-c2cncc(OCC)n2)cc1C ; *c1ccc(-c2cncc(C3CC3)n2)cn1 ; *c1ccc(-c2cncc(Cl)c2)cn1]

or
(f) when A, V, W, R₁, R₄, R₅, R₁₀ and R₁₂ are as follows:

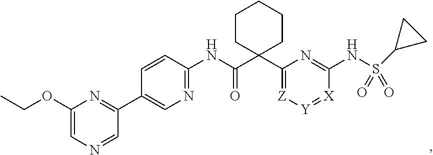

then Z, X and Y are

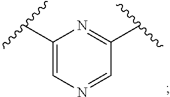 [SMILES: *c1cncc(*)n1]

or
(g) when A, V, W, R₁, R₄, R₅, R₁₀ and R₁₂ are as follows:

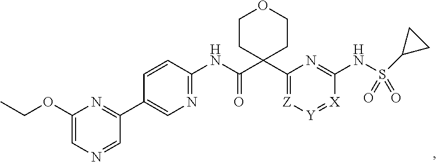

then Z, X and Y are

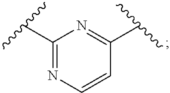 [SMILES: *c1ccnc(*)n1]

or
(h) when A, V, W, R₁, R₄, R₅, R₁₀ and R₁₂ are as follows

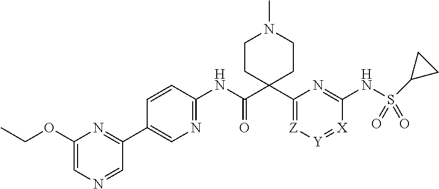

then Z, X and Y are

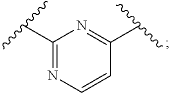 [SMILES: *c1ccnc(*)n1]

or a pharmaceutically acceptable salt and/or pharmaceutically acceptable solvate thereof.
Clause 27. The CTPS1 inhibitor, BCL2 inhibitor, use, method, composition or kit according to clause 26, wherein the CTPS1 inhibitor is selected from the compounds disclosed in List E or a pharmaceutically acceptable salt and/or pharmaceutically acceptable solvate thereof.
Clause 28. The CTPS1 inhibitor, BCL2 inhibitor, use, method, composition or kit according to any one of clauses 1 to 17, wherein the CTPS1 inhibitor is a compound of formula (VI):

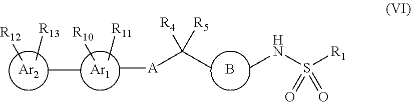

wherein ring B is selected from the group consisting of:

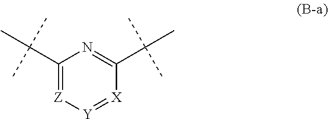

wherein X, Y and Z are as defined below; and

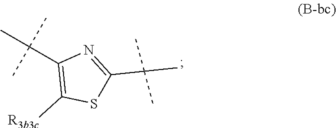

wherein R_{3b3}c is R_{3b} or R_{3c} as defined below;
wherein when B is (B-a) the compound of formula (VI) is a compound of formula (VI-a):

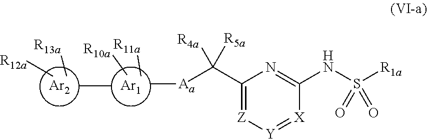

wherein:
Aₐ is Aₐₐ or A_{ba};
   wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂a;
Z is N or CR₃a,
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl,
   or R_{4ba} and R_{5ba} together with the carbon atom to which they are attached form a C₃₋₆cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
wherein
   R₁ₐ is R₁ₐₐ; and/or
   R₄ₐ and R₅ₐ are R₄ₐₐ and R₅ₐₐ; and/or
   Aₐ is Aₐₐ; and
wherein when B is (B-bc) and R_{3b3}c is R_{3b}, the compound of formula (VI) is a compound of formula (VI-b):

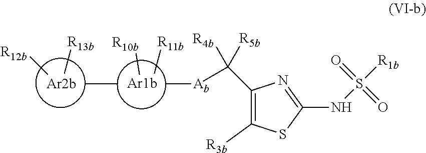

wherein:
A_{b} is A_{ab} or A_{bb};
   wherein:
      A_{ab} is —NR_{6b}CH₂— or —NR_{6b}—;
      A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl; R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1 b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1 b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
   R_{1b} is R_{1ab}; and/or
   R_{4b} and R_{5b} are R_{4ab} and R_{5ab}; and/or
   A is A_{ab}; or
wherein when B is (B-bc) and R_{3b3c} is R_{3c}, the compound of formula (VI) is a compound of formula (VI-c):

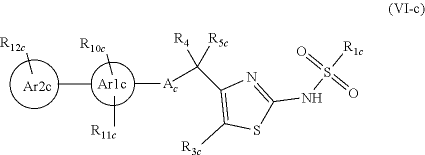

wherein:
   A_{c} is A_{ac} or A_{bc};
      wherein:
      A_{ac} is —CH₂NR_{6c}—;
      A_{bc} is —C(═O)NR_{6c}—;
   R_{1c} is R_{1ac} or R_{1bc};
      wherein:
      R_{1ac} is NR_{32c}R_{33c};
   R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
   R_{3c} is H, CH₃, halo, OC₁₋₂alkyl or CF₃;
   R_{4c} and R_{5c} are either R_{4ac} and R_{5ac} or R_{4bc} and R_{5bc};
   wherein:
      R_{4ac} and R_{5ac} together with the carbon atom to which they are attached form a C₃₋₆cycloalkyl which is:
         substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
         one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
      R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
      R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
      R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
      or R_{4bc} and R_{5bc} together with the carbon atom to which they are attached form a C₃₋₆cycloalkyl or C₃₋₆heterocycloalkyl ring;
   R_{6c} is H or C₁₋₃alkyl;
   Ar1c is a 6-membered aryl or heteroaryl;
   Ar2c is a 6-membered aryl or heteroaryl and is attached to Ar1c in the para position relative to group A_{c};
   R_{10c} is H, halo, C₁₋₃alkyl, OC₁₋₂alkyl, C₁₋₂haloalkyl, OC₁₋₂haloalkyl or CN; R₁₁ₑ is H, F, Cl, CH₃, ethyl, OCH₃, CF₃, OCF₃ or CN;
   R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
   R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
   R_{22c} is H or CH₃;
   R_{23c} is H or C₁₋₂alkyl;
   R_{24c} is H or C₁₋₂alkyl;
   R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
   R_{32c} is C₁₋₃alkyl and R_{33c} is C₁₋₃alkyl; or
   R_{32c} and R_{33c} together with the nitrogen atom to which they are attached form a C₃₋₅heterocycloalkyl;
   wherein:
   R_{1c} is R_{1ac}; and/or
   R_{4c} and R_{5c} are R_{4ac} and R_{5ac}; and/or A_{c} is A_{ac};
      or a pharmaceutically acceptable salt and/or pharmaceutically acceptable solvate thereof.
      Clause 29. The CTPS1 inhibitor, BCL2 inhibitor, use, method, composition or kit according to clause 28, wherein the CTPS1 inhibitor is selected from the compounds disclosed in List F or a pharmaceutically acceptable salt and/or pharmaceutically acceptable solvate thereof.
      Clause 30. The CTPS1 inhibitor, BCL2 inhibitor, use, method, composition or kit according to any one of clauses 1 to 17, wherein the CTPS1 inhibitor is a compound of formula (VII):

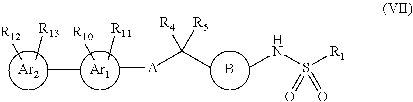

wherein
   A is Aₐ or A_{b};
   wherein
      Aₐ is an amine linker having the following structure: —NH—, —CH₂NH— or —NHCH₂—;
      A_{b} is an amide linker having the following structure: —C(═O)NH— or —NHC(═O)—;
   B is

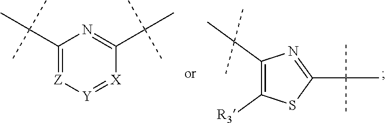

X is N or CH;
Y is N or CR₂;
Z is N or CR₃,
   with the proviso that when at least one of X or Z is N, Y cannot be N;
R₁ is C₁₋₅fluoroalkyl, with the proviso that R₁ is not CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R_{3′} is H, halo, CH₃, OC₁₋₂alkyl or CF₃; and
   when A is —NHC(═O)—, additionally R₃, together with R₅ forms a 5- or 6-membered cycloalkyl or 5 or 6 membered oxygen-containing heterocycloalkyl;
R₄ and R₅ are R₄ₐ and Rea, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆ heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—:
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—:
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;
   or a pharmaceutically acceptable salt and/or pharmaceutically acceptable solvate thereof.
   Clause 31. The CTPS1 inhibitor, BCL2 inhibitor, use, method, composition or kit according to clause 30, wherein the CTPS1 inhibitor is selected from the compounds disclosed in List G or a pharmaceutically acceptable salt and/or pharmaceutically acceptable solvate thereof.
   Clause 32. The CTPS1 inhibitor, BCL2 inhibitor, use, method, composition or kit according to any one of clauses 1 to 17, wherein the CTPS1 inhibitor is compound of formula (VIII):

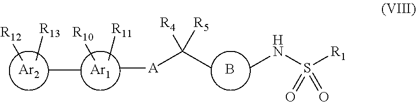

wherein
   A is Aₐ or A_{b};
   wherein
      Aₐ is an amine linker having the following structure: —NH—, —CH₂NH— or —NHCH₂—;
      A_{b} is an amide linker having the following structure: —C(═O)NH— or —NHC(═O)—;
   B is

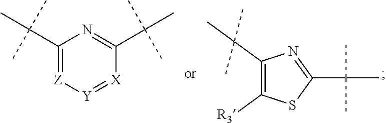

X is N or CH;
Y is N or CR₂;
Z is N or CR₃,
   with the proviso that when at least one of X or Z is N, Y cannot be N; R₁ is C₁₋₅alkyl or C₀₋₂alkyleneC₃₋₅cycloalkyl, which alkyl or (alkylene)cycloalkyl is substituted by CN;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R_{3′} is H, halo, CH₃, OC₁₋₂alkyl or CF₃; and
   when A is —NHC(═O)—, additionally R₃ together with R₅ forms a 5- or 6-membered cycloalkyl or 5 or 6 membered oxygen-containing heterocycloalkyl;
R₄ and R₅ are R₄ₐ and Rea, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the CO₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—:
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—:
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;
   or a pharmaceutically acceptable salt and/or pharmaceutically acceptable solvate thereof.
   Clause 33. The CTPS1 inhibitor, BCL2 inhibitor, use, method, composition or kit according to clause 32, wherein the CTPS1 inhibitor is selected from the compounds disclosed in List H or a pharmaceutically acceptable salt and/or pharmaceutically acceptable solvate thereof.
   Clause 34. The CTPS1 inhibitor, BCL2 inhibitor, use, method, composition or kit according to any one of clauses 1 to 17, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide:

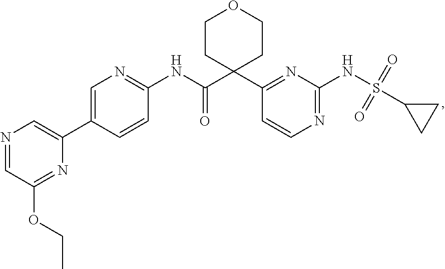 [SMILES: CCOc1cncc(-c2ccc(NC(=O)C3(c4ccnc(NS(=O)(=O)C5CC5)n4)CCOCC3)nc2)n1]

or a pharmaceutically acceptable salt and/or pharmaceutically acceptable solvate thereof.
Clause 35. The CTPS1 inhibitor, BCL2 inhibitor, use, method, composition or kit according to any one of clauses 1 to 17, wherein the CTPS1 inhibitor is N-(5-(6-ethoxypyrazin-2-yl)pyridin-2-yl)-4-(2-(ethylsulfonamido)pyrimidin-4-yl)tetrahydro-2H-pyran-4-carboxamide:

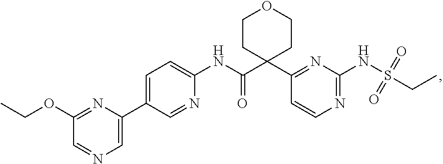 [SMILES: CCOc1cncc(-c2ccc(NC(=O)C3(c4ccnc(NS(=O)(=O)CC)n4)CCOCC3)nc2)n1]

or a pharmaceutically acceptable salt and/or pharmaceutically acceptable solvate thereof.
Clause 36. The CTPS1 inhibitor, BCL2 inhibitor, use, method, composition or kit according to any one of clauses 1 to 35, wherein the CTPS1 inhibitor is in its free form.
Clause 37. The CTPS1 inhibitor, BCL2 inhibitor, use, method, composition or kit according to any one of clauses 1 to 35, wherein the CTPS1 inhibitor is a pharmaceutically acceptable salt.
Clause 38. The CTPS1 inhibitor, BCL2 inhibitor, use, method, composition or kit according to any one of clauses 1 to 35, wherein the CTPS1 inhibitor is a pharmaceutically acceptable solvate.
Clause 39. The CTPS1 inhibitor, BCL2 inhibitor, use, method, composition or kit according to any one of clauses 1 to 35, wherein the CTPS1 inhibitor is a pharmaceutically acceptable salt and a pharmaceutically acceptable solvate.
Clause 40. The CTPS1 inhibitor, BCL2 inhibitor, use, method, composition or kit according to any one of clauses 1 to 17, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide (‘CTPS1-IA’):

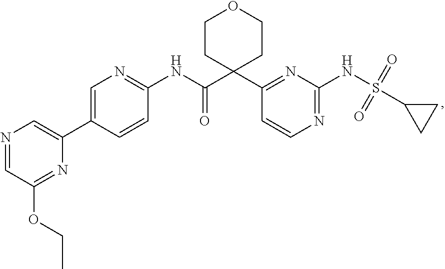 [SMILES: CCOc1cncc(-c2ccc(NC(=O)C3(c4ccnc(NS(=O)(=O)C5CC5)n4)CCOCC3)nc2)n1]

or a pharmaceutically acceptable salt thereof.
Clause 41. The CTPS1 inhibitor, BCL2 inhibitor, use, method, composition or kit according to any one of clauses 1 to 17, wherein the CTPS1 inhibitor is N-(5-(6-ethoxypyrazin-2-yl)pyridin-2-yl)-4-(2-(ethylsulfonamido)pyrimidin-4-yl)tetrahydro-2H-pyran-4-carboxamide (‘CTPS1-IB’):

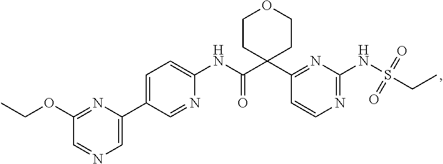 [SMILES: CCOc1cncc(-c2ccc(NC(=O)C3(c4ccnc(NS(=O)(=O)CC)n4)CCOCC3)nc2)n1]

or a pharmaceutically acceptable salt thereof.
Clause 42. The CTPS1 inhibitor, BCL2 inhibitor, use, method, composition or kit according to any one of clauses 1 to 41, wherein the CTPS1 inhibitor is provided in a natural isotopic form.
Clause 43. The CTPS1 inhibitor, BCL2 inhibitor, use, method, composition or kit according to any one of clauses 1 to 42, wherein the BCL2 inhibitor has a Ki value for binding to human BCL2 of 50 nM or lower.
Clause 44. The CTPS1 inhibitor, BCL2 inhibitor, use, method, composition or kit according to clause 43, wherein the BCL2 inhibitor has a Ki value for binding to human BCL2 of 20 nM or lower.
Clause 45. The CTPS1 inhibitor, BCL2 inhibitor, use, method, composition or kit according to clause 44, wherein the BCL2 inhibitor has a Ki value for binding to human BCL2 of 10 nM or lower.
Clause 46. The CTPS1 inhibitor, BCL2 inhibitor, use, method, composition or kit according to clause 45, wherein the BCL2 inhibitor has a Ki value for binding to human BCL2 of 5 nM or lower.
Clause 47. The CTPS1 inhibitor, BCL2 inhibitor, use, method, composition or kit according to clause 46, wherein the BCL2 inhibitor has a Ki value for binding to human BCL2 of 1 nM or lower.
Clause 48. The CTPS1 inhibitor, BCL2 inhibitor, use, method, composition or kit according to any one of clauses 43 to 47, wherein the Ki value of the BCL2 inhibitor for binding to human BCL2 is established using the assay procedure set out in Example 3.
Clause 49. The CTPS1 inhibitor, BCL2 inhibitor, use, method, composition or kit according to any one of clauses 1 to 48, wherein the BCL2 inhibitor has a selectivity for human BCL2 over human BCL2L1 of >2-fold.
Clause 50. The CTPS1 inhibitor, BCL2 inhibitor, use, method, composition or kit according to clause 49, wherein the BCL2 inhibitor has a selectivity for human BCL2 over human BCL2L1 of >5-fold.
Clause 51. The CTPS1 inhibitor, BCL2 inhibitor, use, method, composition or kit according to clause 50, wherein the BCL2 inhibitor has a selectivity for human BCL2 over human BCL2L1 of >10-fold.
Clause 52. The CTPS1 inhibitor, BCL2 inhibitor, use, method, composition or kit according to any one of clauses 1 to 51, wherein the BCL2 inhibitor has a selectivity for human BCL2 over human MCL1 of >2-fold.
Clause 53. The CTPS1 inhibitor, BCL2 inhibitor, use, method, composition or kit according to clause 52, wherein the BCL2 inhibitor has a selectivity for human BCL2 over human MCL1 of >5-fold.
Clause 54. The CTPS1 inhibitor, BCL2 inhibitor, use, method, composition or kit according to clause 53, wherein the BCL2 inhibitor has a selectivity for human BCL2 over human MCL1 of >10-fold.
Clause 55. The CTPS1 inhibitor, BCL2 inhibitor, use, method, composition or kit according to any one of clauses 49 to 54, wherein the selectivity of the BCL2 inhibitor is established using the assay procedure set out in Example 4.
Clause 56. The CTPS1 inhibitor, BCL2 inhibitor, use, method, composition or kit according to any one of clauses 1 to 55, wherein the BCL2 inhibitor is a BH-3 mimetic.
Clause 57. The CTPS1 inhibitor, BCL2 inhibitor, use, method, composition or kit according to any one of clauses 1 to 55, wherein the BCL2 inhibitor is selected from venetoclax, navitoclax, obatoclax, BCL201 and AT101, pharmaceutically acceptable salts thereof and/or pharmaceutically acceptable solvates thereof.
Clause 58. The CTPS1 inhibitor, BCL2 inhibitor, use, method, composition or kit according to clause 57, wherein the BCL2 inhibitor is selected from venetoclax, navitoclax, obatoclax and BCL201, pharmaceutically acceptable salts thereof and/or pharmaceutically acceptable solvates thereof.
Clause 59. The CTPS1 inhibitor, BCL2 inhibitor, use, method, composition or kit according to clause 58, wherein the BCL2 inhibitor is venetoclax:

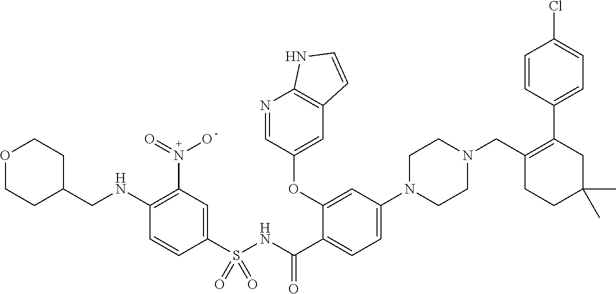

(4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide), pharmaceutically acceptable salts thereof and/or pharmaceutically acceptable solvates thereof.
Clause 60. The CTPS1 inhibitor, BCL2 inhibitor, use, method, composition or kit according to clause 58, wherein the BCL2 inhibitor is navitoclax:

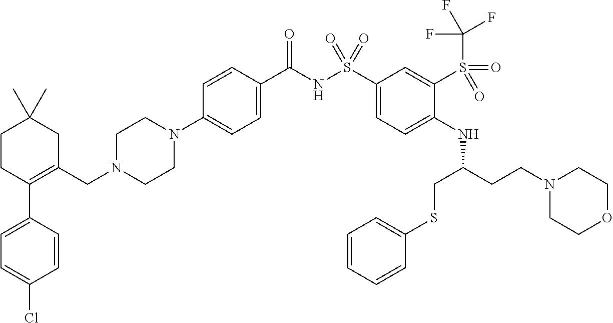 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

(4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide), pharmaceutically acceptable salts thereof and/or pharmaceutically acceptable solvates thereof.
Clause 61. The CTPS1 inhibitor, BCL2 inhibitor, use, method, composition or kit according to clause 58, wherein the BCL2 inhibitor is obatoclax:

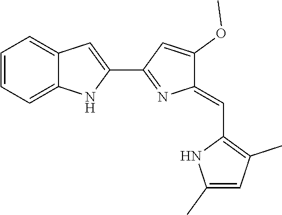 [SMILES: COC1=CC(c2cc3ccccc3[nH]2)=N/C1=C\c1[nH]c(C)cc1C]

((2Z)-2-[(5Z)-5-[(3,5-dimethyl-1H-pyrrol-2-yl)methylidene]-4-methoxypyrrol-2-ylidene]indole), pharmaceutically acceptable salts thereof and/or pharmaceutically acceptable solvates thereof.
Clause 62. The CTPS1 inhibitor, BCL2 inhibitor, use, method, composition or kit according to clause 58, wherein the BCL2 inhibitor is BCL201:

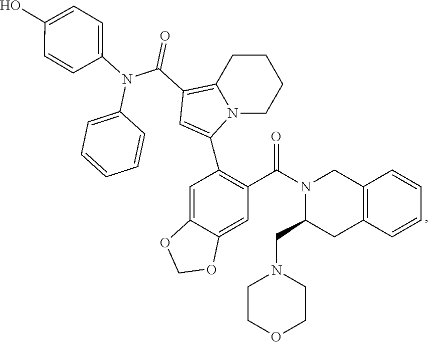 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

pharmaceutically acceptable salts thereof and/or pharmaceutically acceptable solvates thereof.
Clause 63. The CTPS1 inhibitor, BCL2 inhibitor, use, method, composition or kit according to clause 58, wherein the BCL2 inhibitor is AT101:

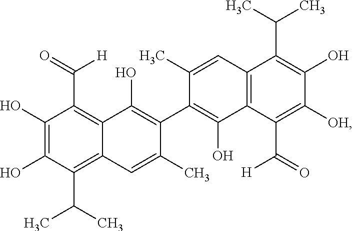 [SMILES: Cc1cc2c(C(C)C)c(O)c(O)c(C=O)c2c(O)c1-c1c(C)cc2c(C(C)C)c(O)c(O)c(C=O)c2c1O]

pharmaceutically acceptable salts thereof and/or pharmaceutically acceptable solvates thereof.
Clause 64. The CTPS1 inhibitor, BCL2 inhibitor, use, method, composition or kit according to any one of clauses 1 to 63, wherein the BCL2 inhibitor is in its free form.
Clause 65. The CTPS1 inhibitor, BCL2 inhibitor, use, method, composition or kit according to any one of clauses 1 to 63, wherein the BCL2 inhibitor is a pharmaceutically acceptable salt.
Clause 66. The CTPS1 inhibitor, BCL2 inhibitor, use, method, composition or kit according to any one of clauses 1 to 63, wherein the BCL2 inhibitor is a pharmaceutically acceptable solvate.
Clause 67. The CTPS1 inhibitor, BCL2 inhibitor, use, method, composition or kit according to any one of clauses 1 to 63, wherein the BCL2 inhibitor is a pharmaceutically acceptable salt and a pharmaceutically acceptable solvate.
Clause 68. The CTPS1 inhibitor, BCL2 inhibitor, use, method, composition or kit according to clause 59, wherein the BCL2 inhibitor is venetoclax:

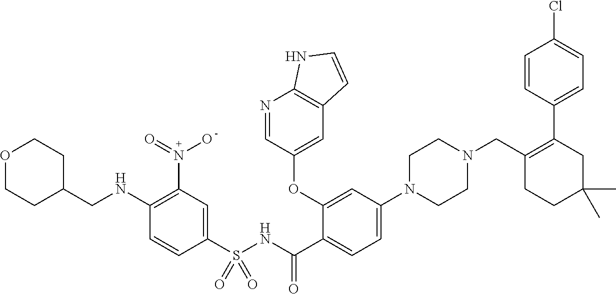

(4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide).
Clause 69. The CTPS1 inhibitor, BCL2 inhibitor, use, method, composition or kit according to clause 68, wherein the CTPS1 inhibitor is CTPS-IA or a pharmaceutically acceptable salt and/or pharmaceutically acceptable solvate thereof and the BCL2 inhibitor is venetoclax.
Clause 70. The CTPS1 inhibitor, BCL2 inhibitor, use, method, composition or kit according to clause 69, wherein the CTPS1 inhibitor is CTPS-IA or a pharmaceutically acceptable salt thereof and the BCL2 inhibitor is venetoclax.
Clause 71. The CTPS1 inhibitor, BCL2 inhibitor, use, method, composition or kit according to any one of clauses 1 to 70, wherein the BCL2 inhibitor is provided in a natural isotopic form.
Clause 72. The CTPS1 inhibitor, BCL2 inhibitor, use, method, composition or kit according to any one of clauses 1 to 71 wherein the CTPS1 inhibitor and the BCL2 inhibitor act synergistically in treating the cancer.
Clause 73. The CTPS1 inhibitor, BCL2 inhibitor, use, method, composition or kit according to clause 72 wherein the combined administration of the CTPS1 inhibitor and the BCL2 inhibitor results in a beneficial effect greater than the sum of the beneficial effects of each agent administered alone.
Clause 74. The CTPS1 inhibitor, BCL2 inhibitor, use, method, composition or kit according to either clause 72 or 73 wherein the CTPS1 inhibitor and the BCL2 inhibitor achieve a Bliss score (Bliss 1939; Zheng 2021) of >10 when applied to a cancer cell line as set out in Example 6.
Clause 75. The CTPS1 inhibitor, BCL2 inhibitor, use, method, composition or kit according to any one of clauses 1 to 74, wherein the BCL2 inhibitor and the CTPS1 inhibitor are administered to a mammal.
Clause 76. The CTPS1 inhibitor, BCL2 inhibitor, use, method, composition or kit according to clause 75, wherein the BCL2 inhibitor and the CTPS1 inhibitor are administered to a human.
Clause 77. The CTPS1 inhibitor, BCL2 inhibitor, use, method, composition or kit according to any one of clauses 1 to 76, wherein the CTPS1 inhibitor and the BCL2 inhibitor are separately formulated.
Clause 78. The CTPS1 inhibitor, BCL2 inhibitor, use, method, composition or kit according to clause 77, wherein the CTPS1 inhibitor and the BCL2 inhibitor are administered separately.
Clause 79. The CTPS1 inhibitor, BCL2 inhibitor, use, method, composition or kit according to any one of clauses 1 to 77, wherein the CTPS1 inhibitor and the BCL2 inhibitor are administered simultaneously.
Clause 80. The CTPS1 inhibitor, BCL2 inhibitor, use, method, composition or kit according to clause 79, wherein the CTPS1 inhibitor and the BCL2 inhibitor are co-formulated.
Clause 81. The CTPS1 inhibitor, BCL2 inhibitor, use, method, composition or kit according to any one of clauses 1 to 80, wherein the CTPS1 inhibitor is administered by oral, parenteral, buccal, sublingual, nasal or rectal administration.
Clause 82. The CTPS1 inhibitor, BCL2 inhibitor, use, method, composition or kit according to clause 81, wherein the CTPS1 inhibitor is administered orally.
Clause 83. The CTPS1 inhibitor, BCL2 inhibitor, use, method, composition or kit according to any one of clauses 1 to 82, wherein the BCL2 inhibitor is administered by oral, parenteral, buccal, sublingual, nasal or rectal administration.
Clause 84. The CTPS1 inhibitor, BCL2 inhibitor, use, method, composition or kit according to clause 83, wherein the BCL2 inhibitor is administered orally.
Clause 85. The CTPS1 inhibitor, BCL2 inhibitor, use, method, composition or kit according to any one of clauses 1 to 84, wherein the CTPS1 inhibitor and BCL2 inhibitor administered separately, sequentially or simultaneously with one or more further pharmaceutically acceptable active ingredients.
Clause 86. The CTPS1 inhibitor, BCL2 inhibitor, use, method, composition or kit according to clause 85, wherein the one or more further pharmaceutically acceptable active ingredients are selected from tyrosine kinase inhibitors such as, for example, axitinib, dasatinib, erlotinib, imatinib, nilotinib, pazopanib and sunitinib.
Clause 87. The CTPS1 inhibitor, BCL2 inhibitor, use, method, composition or kit according to clause 85, wherein the one or more further pharmaceutically acceptable active ingredients are selected from zacitidine, decitabine, or cytarabine.
Clause 88. The CTPS1 inhibitor, BCL2 inhibitor, use, method, composition or kit according to clause 85, wherein the one or more further pharmaceutically acceptable active ingredients are selected from anticancer antibodies, such as those selected from the group consisting of anti-CD20 antibodies (such as obinutuzumab, ofatumumab, tositumomab or rituximab) or other antibodies such as olaratumab, daratumumab, necitumumab, dinutuximab, traztuzumab emtansine, pertuzumab, brentuximab, panitumumab, catumaxomab, bevacizumab, cetuximab, traztuzumab and gentuzumab ozogamycin.
Clause 89. The CTPS1 inhibitor, BCL2 inhibitor, use, method, composition or kit according to any one of clauses 1 to 88, administered in combination with radiotherapy.
Clause 90. The CTPS1 inhibitor, BCL2 inhibitor, use, method, composition or kit according to any one of clauses 1 to 89, administered in combination with surgery.
Clause 91. The CTPS1 inhibitor, BCL2 inhibitor, use, method, composition or kit according to any one of clauses 1 to 90, administered in combination with hyperthermia therapy.
Clause 92. The CTPS1 inhibitor, BCL2 inhibitor, use, method, composition or kit according to any one of clauses 1 to 91, administered in combination with cryotherapy.
Clause 93. The CTPS1 inhibitor, BCL2 inhibitor, use, method, composition or kit according to any one of clauses 1 to 92, wherein the cancer is a cancer displaying high replicative stress.
Clause 94. The CTPS1 inhibitor, BCL2 inhibitor, use, method, composition or kit according to any one of clauses 1 to 93, wherein the cancer is a cancer displaying high expression of BCL2.
Clause 95. The CTPS1 inhibitor, BCL2 inhibitor, use, method, composition or kit according to any one of clauses 1 to 94, wherein the cancer is a cancer displaying a high BCL2:BCL2L1 ratio.
Clause 96. The CTPS1 inhibitor, BCL2 inhibitor, use, method, composition or kit according to any one of clauses 1 to 95, wherein the cancer is a cancer displaying a high BCL2:MCL1 ratio.
Clause 97. The CTPS1 inhibitor, BCL2 inhibitor, use, method, composition or kit according to any one of clauses 1 to 96, wherein the cancer constitutively expresses c-myc.
Clause 98. The CTPS1 inhibitor, BCL2 inhibitor, use, method, composition or kit according to any one of clauses 1 to 97, wherein the cancer is a non-haematological cancer.
Clause 99. The CTPS1 inhibitor, BCL2 inhibitor, use, method, composition or kit according to clause 98, wherein the cancer is selected from the group consisting of bladder cancer, breast, melanoma, neuroblastoma, malignant pleural mesothelioma, and sarcoma.
Clause 100. The CTPS1 inhibitor, BCL2 inhibitor, use, method, composition or kit according to either clause 98 or 99, wherein the cancer is a solid tumour.
Clause 101. The CTPS1 inhibitor, BCL2 inhibitor, use, method, composition or kit according to any one of clauses 1 to 97, wherein the cancer is a haematological cancer.
Clause 102. The CTPS1 inhibitor, BCL2 inhibitor, use, method, composition or kit according to clause 101, wherein the haematological cancer is selected from the list consisting of acute myeloid leukemia, angioimmunoblastic T-cell lymphoma, B-cell acute lymphoblastic leukemia, Sweet syndrome, T-cell non-Hodgkin lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell non-Hodgkin lymphoma (including Burkitt lymphoma, diffuse large B-cell lymphoma, follicular lymphoma, mantle cell lymphoma, marginal zone lymphoma), hairy cell leukemia, Hodgkin lymphoma, lymphoblastic lymphoma, lymphoplasmacytic lymphoma, mucosa-associated lymphoid tissue lymphoma, multiple myeloma, myelodysplastic syndrome, plasma cell myeloma, primary mediastinal large B-cell lymphoma, chronic myeloproliferative disorders (such as chronic myeloid leukemia, primary myelofibrosis, essential thrombocythemia, polycythemia vera) or chronic lymphocytic leukemia.
Clause 103. The CTPS1 inhibitor, BCL2 inhibitor, use, method, composition or kit according to clause 102, wherein the haematological cancer is selected from the list consisting of B-cell non-Hodgkin lymphoma (including Burkitt lymphoma, diffuse large B-cell lymphoma, follicular lymphoma, mantle cell lymphoma, marginal zone lymphoma), multiple myeloma and plasma cell leukaemia.
Clause 104. The CTPS1 inhibitor, BCL2 inhibitor, use, method, composition or kit according to clause 103, wherein the haematological cancer is selected from the list consisting of T cell lymphoma, diffuse large B cell lymphoma, plasma cell myeloma, acute myeloid leukaemia, chronic lymphocytic leukaemia or peripheral T cell lymphoma.
Clause 105. The CTPS1 inhibitor, BCL2 inhibitor, use, method, composition or kit according to clause 104, wherein the haematological cancer is T cell lymphoma.
Clause 106. The CTPS1 inhibitor, BCL2 inhibitor, use, method, composition or kit according to clause 104, wherein the haematological cancer is diffuse large B cell lymphoma.
Clause 107. The CTPS1 inhibitor, BCL2 inhibitor, use, method, composition or kit according to clause 104, wherein the haematological cancer is plasma cell myeloma.
Clause 108. The CTPS1 inhibitor, BCL2 inhibitor, use, method, composition or kit according to clause 104, wherein the haematological cancer is acute myeloid leukaemia.
Clause 109. The CTPS1 inhibitor, BCL2 inhibitor, use, method, composition or kit according to clause 104, wherein the haematological cancer is chronic lymphocytic leukaemia.
Clause 110. The CTPS1 inhibitor, BCL2 inhibitor, use, method, composition or kit according to clause 104, wherein the haematological cancer is peripheral T cell lymphoma.
Clause 111. The CTPS1 inhibitor, BCL2 inhibitor, use, method, composition or kit according to clause 101, wherein the haematological cancer is T-cell prolymphocytic leukemia.
Clause 112. The CTPS1 inhibitor, BCL2 inhibitor, use, method, composition or kit according to any one of clauses 1 to 97, wherein the cancer is selected from the list consisting of leukemia, lymphoma, lung cancer, bone cancer, melanoma, prostate cancer, brain tumours, colorectal cancer, esophagogastric cancer, breast cancer, endometrial cancer renal cancer, sarcoma, hepatic cancer, pancreatic cancer, bladder cancer, thyroid cancer, ovarian cancer, head and neck cancer, mesothelioma and biliary tract cancer.
Clause 113. The CTPS1 inhibitor, BCL2 inhibitor, use, method, composition or kit according to clause 112, wherein the cancer is selected from the list consisting of leukemia, lymphoma, lung cancer, bone cancer, melanoma, prostate cancer, brain tumours, colorectal cancer, esophagogastric cancer, breast cancer, endometrial cancer renal cancer, sarcoma, hepatic cancer and pancreatic cancer.
Clause 114. The CTPS1 inhibitor, BCL2 inhibitor, use, method, composition or kit according to clause 113, wherein the cancer is selected from the list consisting of leukemia, lymphoma, lung cancer, bone cancer, melanoma, prostate cancer, brain tumours and colorectal cancer.
Clause 115. The CTPS1 inhibitor, BCL2 inhibitor, use, method, composition or kit according to any one of clauses 1 to 114, for administration to a subject identified as having a cancer expected to be susceptible to treatment by a CTPS1 inhibitor and a BCL2 inhibitor.
Clause 116. The CTPS1 inhibitor, BCL2 inhibitor, use, method, composition or kit according to any one of clauses 1 to 114, for administration to a subject from whom a sample of cancer cells has been shown to be susceptible to treatment by a CTPS1 inhibitor and a BCL2 inhibitor.
Clause 117. The CTPS1 inhibitor, BCL2 inhibitor, use, method, composition or kit according to any one of clauses 1 to 116, wherein the CTPS1 inhibitor and BCL2 inhibitor are administered orally.
Clause 118. The CTPS1 inhibitor, BCL2 inhibitor, use, method, composition or kit according to any one of clauses 1 to 117, wherein the CTPS1 inhibitor is in a solid pharmaceutical composition.
Clause 119. The CTPS1 inhibitor, BCL2 inhibitor, use, method, composition or kit according to any one of clauses 1 to 118, wherein the BCL2 inhibitor is in a solid pharmaceutical composition.
Clause 120. The CTPS1 inhibitor, BCL2 inhibitor, use, method, composition or kit according to any one of clauses 1 to 119, wherein the CTPS1 inhibitor is in a solid pharmaceutical composition and the BCL2 inhibitor is in a solid pharmaceutical composition.
Clause 121. The CTPS1 inhibitor, BCL2 inhibitor, use, method, composition or kit according to any one of clauses 1 to 120, wherein the CTPS1 inhibitor is administered orally in a solid pharmaceutical composition.
Clause 122. The CTPS1 inhibitor, BCL2 inhibitor, use, method, composition or kit according to any one of clauses 1 to 121, wherein the BCL2 inhibitor is administered orally in a solid pharmaceutical composition.
Clause 123. The CTPS1 inhibitor, BCL2 inhibitor, use, method, composition or kit according to any one of clauses 1 to 117, wherein the CTPS1 inhibitor is administered orally in a solid pharmaceutical composition and the BCL2 inhibitor is administered orally in a solid pharmaceutical composition.
Clause 124. The CTPS1 inhibitor, BCL2 inhibitor, use, method, composition or kit according to any one of clauses 1 to 120, wherein the BCL2 inhibitor is administered at a daily dose of up to 600 mg, suitably a daily dose of 1.0 mg to 500 mg.
Clause 125. The CTPS1 inhibitor, BCL2 inhibitor, use, method, composition or kit according to any one of clauses 1 to 121, wherein the CTPS1 inhibitor is:
N-(5-(6-ethoxypyrazin-2-yl)pyridin-2-yl)-4-(2-(ethylsulfonamido)pyrimidin-4-yl)tetrahydro-2H-pyran-4-carboxamide

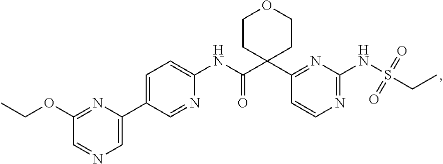 [SMILES: CCOc1cncc(-c2ccc(NC(=O)C3(c4ccnc(NS(=O)(=O)CC)n4)CCOCC3)nc2)n1]

or a pharmaceutically acceptable salt and/or pharmaceutically acceptable solvate thereof; and the BCL2 inhibitor is venetoclax, or a pharmaceutically acceptable salt and/or pharmaceutically acceptable solvate thereof.
Clause 126. The CTPS1 inhibitor, BCL2 inhibitor, use, method, composition or kit according to any one of clauses 1 to 121, wherein the CTPS1 inhibitor is:
4-(2-(cyclopropanesulfonamido)pyrimidin-4-yl)-N-(5-(6-ethoxypyrazin-2-yl)pyridin-2-yl)tetrahydro-2H-pyran-4-carboxamide:

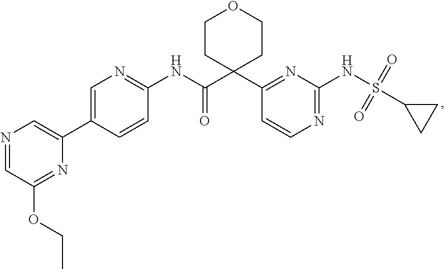 [SMILES: CCOc1cncc(-c2ccc(NC(=O)C3(c4ccnc(NS(=O)(=O)C5CC5)n4)CCOCC3)nc2)n1]

or a pharmaceutically acceptable salt and/or pharmaceutically acceptable solvate thereof; and the BCL2 inhibitor is venetoclax, or a pharmaceutically acceptable salt and/or pharmaceutically acceptable solvate thereof.
Clause 127. The CTPS1 inhibitor, BCL2 inhibitor, use, method, composition or kit according to any one of clauses 1 to 124, wherein the BLC2 inhibitor is not venetoclax.
Clause 128. The CTPS1 inhibitor, BCL2 inhibitor, use, method, composition or kit according to clause 127, wherein the BLC2 inhibitor is not venetoclax, or a pharmaceutically acceptable salt and/or pharmaceutically acceptable solvate thereof.
Clause 129. The CTPS1 inhibitor, BCL2 inhibitor, use, method, composition or kit according to any one of clauses 1 to 128, wherein the CTPS1 inhibitor is not a CTPS1 inhibitor as defined in claim 1 of WO2022/087634.
Clause 130. The CTPS1 inhibitor, BCL2 inhibitor, use, method, composition or kit according to any one of clauses 1 to 128, wherein the CTPS1 inhibitor is not a CTPS1 inhibitor as defined in WO2022/087634.
Clause 131. The CTPS1 inhibitor, BCL2 inhibitor, use, method, composition or kit according to any one of clauses 1 to 128, wherein the CTPS1 inhibitor is a CTPS1 inhibitor disclosed in PCT publication number WO2022087634.
Clause 132. The CTPS1 inhibitor, BCL2 inhibitor, use, method, composition or kit according to any one of clauses 1 to 128, wherein the CTPS1 inhibitor is (i) a compound described in any one of claims 1 to 31 of WO2022087634 or a pharmaceutically acceptable salt and/or pharmaceutically acceptable solvate thereof, (ii) a compound selected from compounds 1-1 to 1-286 of WO2022087634, or a pharmaceutically acceptable salt and/or pharmaceutically acceptable solvate thereof, or (iii) a compound selected from compounds Z-1 to Z-10 of WO2022087634, or a pharmaceutically acceptable salt and/or pharmaceutically acceptable solvate thereof.
Clause 133. The CTPS1 inhibitor, BCL2 inhibitor, use, method, composition or kit according to any one of clauses 1 to 128, wherein the CTPS1 inhibitor is not a CTPS1 inhibitor disclosed in PCT publication number WO2022087634.
Clause 134. The CTPS1 inhibitor, BCL2 inhibitor, use, method, composition or kit according to any one of clauses 1 to 128, wherein the CTPS1 inhibitor is not (i) a compound described in any one of claims 1 to 31 of WO2022087634 or a pharmaceutically acceptable salt and/or pharmaceutically acceptable solvate thereof, (ii) a compound selected from compounds 1-1 to 1-286 of WO2022087634, or a pharmaceutically acceptable salt and/or pharmaceutically acceptable solvate thereof, or (iii) a compound selected from compounds Z-1 to Z-10 of WO2022087634, or a pharmaceutically acceptable salt and/or pharmaceutically acceptable solvate thereof.

### REFERENCES

Anderson et al. Blood. 2016 Jun. 23; 127(25):3215-3224.
Behan et al. Nature. 2019 April; 568(7753):511-516.
B/biogps.org/, EMBL-EBI Expression s://www.ebi.ac.uk/gxa/home
Bliss Ann. Appl. Biol. 1939 26, 585-615.
Cancer Dependenc/depmap.org/
Casara et al. Oncotarget. 2018 Apr. 13; 9(28):20075-20088.
CellMineiscover.nci.nih.gov/rsconnect/cellminercdb/
Diepstraten et al. Nat Rev Cancer. 2022 January; 22(1):45-64.
Doepner et al. Nature. 2019 April; 568(7753):511-516.
EMBL-EBI Expression s://www.ebi.ac.uk/gxa/home
Li et al. Cancer Chemother Pharmacol. 2008 March; 61(3):525-34.
Martin et al. Nature. 2014 Jun. 12; 510(7504):288-292.
Martin et al. JCI Insight. 2020 Mar. 12; 5(5):e133880.
Park et al. J Med Chem. 2008 Nov. 13; 51(21):6902-6915.
Punnoose et al. Mol Cancer Ther. 2016 May; 15(5):1132-1144.
Roberts et al. Blood. 2021 Sep. 30; 138(13):1120-1136.
Souers et al. Nat Med. 2013 February; 19(2):202-208.
Tse et al. Cancer Res. 2008 May 1; 68(9):3421-3428.
Tutusaus et al. Oncotarget. 2018 Mar. 30; 9(24):16701-16717.
van Kuilenburg et al. Biochim Biophys Acta. 2000 Jul. 24; 1492(2-3):548-552.
Wang et al. J Med Chem. 2006 Oct. 19; 49(21):6139-6142.
Warren et al. Cell Death Dis. 2019 Feb. 21; 10(3):177.
Zhang et al. Anal Biochem. 2002 Aug. 1; 307(1):70-75.
Zheng et al. bioRxiv, 2021.06.01.446564.
WO2019106146
WO2019106156
WO2019179652
WO2019180244
WO2020083975
WO2020245664
WO2020245665
WO2021053402
WO2021053403
WO2022/087634

1-34. (canceled)

35. A pharmaceutical composition comprising a CTPS1 inhibitor and a BCL2 inhibitor.

36. A method of treating cancer in a subject which method comprises administering to the subject a CTPS1 inhibitor and a BCL2 inhibitor.

37. A kit of parts comprising:
a) a first container comprising a CTPS1 inhibitor; and
b) a second container comprising a BCL2 inhibitor.

38. The method according to claim 36, wherein the CTPS1 inhibitor has an IC₅₀ of 10 uM or lower, established using the assay procedure set out in Example 1.

39. The method according to claim 36, wherein the CTPS1 inhibitor has a selectivity for CTPS1 over CTPS2 of at least 2-fold, established using the assay procedure set out in Example 2.

40. The method according to claim 39, wherein the CTPS1 inhibitor has a selectivity for CTPS1 over CTPS2 of at least 2-fold, established using the assay procedure set out in Example 2.

41. The method according to claim 36, wherein the CTPS1 inhibitor is a compound of formula (III): 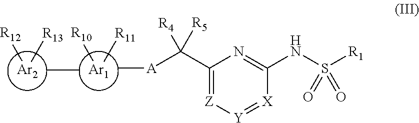
wherein
A is an amide linker having the following structure: —C(═O)NH— or —NHC(═O)—;
X is N or CH;
Y is N or CR₂;
Z is N or CR₃,
with the proviso that when at least one of X or Z is N, Y cannot be N;
R₁ is C₁₋₅alkyl, C₀₋₂alkyleneC₃₋₅cycloalkyl which cycloalkyl is optionally substituted by CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl; and
when A is —NHC(═O)—:
R₄ and R₅ may additionally be selected from halo, OC₁₋₆haloalkyl, OC₀₋₂alkyleneC₃₋₆ cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄ haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
when A is —NHC(═O)—:
R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O⁻);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
or a pharmaceutically acceptable salt and/or pharmaceutically acceptable solvate thereof.

42. The method according to claim 36, wherein the CTPS1 inhibitor is N-(5-(6-ethoxypyrazin-2-yl)pyridin-2-yl)-4-(2-(ethylsulfonamido)pyrimidin-4-yl)tetrahydro-2H-pyran-4-carboxamide: 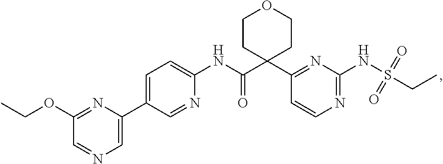 [SMILES: CCOc1cncc(-c2ccc(NC(=O)C3(c4ccnc(NS(=O)(=O)CC)n4)CCOCC3)nc2)n1]
or a pharmaceutically acceptable salt and/or pharmaceutically acceptable solvate thereof.

43. The method according to claim 36, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide: 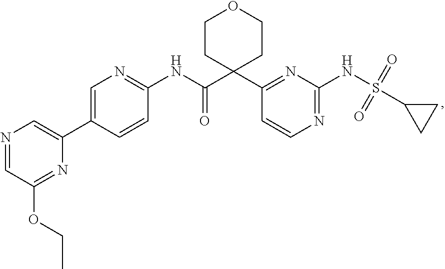 [SMILES: CCOc1cncc(-c2ccc(NC(=O)C3(c4ccnc(NS(=O)(=O)C5CC5)n4)CCOCC3)nc2)n1]
or a pharmaceutically acceptable salt and/or pharmaceutically acceptable solvate thereof.

44. The method according to claim 36, wherein the CTPS1 inhibitor is selected from the compounds disclosed in any one of Lists A to H or a pharmaceutically acceptable salt and/or pharmaceutically acceptable solvate thereof.

45. The method according to claim 36, wherein the BCL2 inhibitor has a Ki value for binding to BCL2 of 50 nM or lower established using the assay procedure set out in Example 3.

46. The method according to claim 38, wherein the BCL2 inhibitor has a Ki value for binding to BCL2 of 50 nM or lower established using the assay procedure set out in Example 3.

47. The method according to claim 39, wherein the BCL2 inhibitor has a Ki value for binding to BCL2 of 50 nM or lower established using the assay procedure set out in Example 3.

48. The method according to claim 42, wherein the BCL2 inhibitor has a Ki value for binding to BCL2 of 50 nM or lower established using the assay procedure set out in Example 3.

49. The method according to claim 43, wherein the BCL2 inhibitor has a Ki value for binding to BCL2 of 50 nM or lower established using the assay procedure set out in Example 3.

50. The method according to claim 36, wherein the BCL2 inhibitor is a BH-3 mimetic.

51. The method according to claim 36, wherein the BCL2 inhibitor is selected from venetoclax, navitoclax, obatoclax, BCL201 and AT101, pharmaceutically acceptable salts thereof and/or pharmaceutically acceptable solvates thereof.

52. The method according to claim 36, wherein the BCL2 inhibitor is venetoclax, pharmaceutically acceptable salts thereof and/or pharmaceutically acceptable solvates thereof.

53. The method according to claim 36, wherein the CTPS1 inhibitor is:
N-(5-(6-ethoxypyrazin-2-yl)pyridin-2-yl)-4-(2-(ethylsulfonamido)pyrimidin-4-yl)tetrahydro-2H-pyran-4-carboxamide: 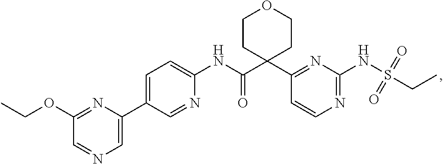 [SMILES: CCOc1cncc(-c2ccc(NC(=O)C3(c4ccnc(NS(=O)(=O)CC)n4)CCOCC3)nc2)n1]
or a pharmaceutically acceptable salt and/or pharmaceutically acceptable solvate thereof; and the BCL2 inhibitor is venetoclax, or a pharmaceutically acceptable salt and/or pharmaceutically acceptable solvate thereof.

54. The method according to claim 36, wherein the CTPS1 inhibitor is:
4-(2-(cyclopropanesulfonamido)pyrimidin-4-yl)-N-(5-(6-ethoxypyrazin-2-yl)pyridin-2-yl)tetrahydro-2H-pyran-4-carboxamide: 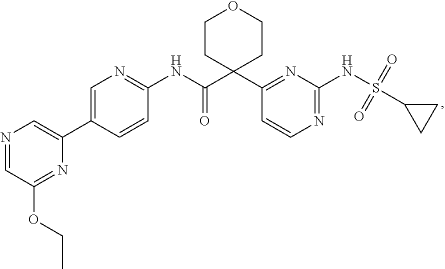 [SMILES: CCOc1cncc(-c2ccc(NC(=O)C3(c4ccnc(NS(=O)(=O)C5CC5)n4)CCOCC3)nc2)n1]
or a pharmaceutically acceptable salt and/or pharmaceutically acceptable solvate thereof; and the BCL2 inhibitor is venetoclax, or a pharmaceutically acceptable salt and/or pharmaceutically acceptable solvate thereof.

55. The method according to claim 36, wherein the cancer is a haematological cancer.
